(19) United States
(12) Patent Application Publication
(10) Pub. No.: US 2004/0087543 A1
(43) Pub. Date: May 6, 2004

(54) Methods and products for mucosal delivery

(76) Inventors: Zachary Shriver, Boston, MA (US); Ganesh Venkataraman, Bedford, MA (US); Mallikarjun Sundaram, Ashland, MA (US); Ram Sasisekharan, Cambridge, MA (US); Thomas Richardson, South Boston, MA (US); Yiwei Qi, Charlestown, MA (US)

Correspondence Address: FISH & RICHARDSON PC, 225 FRANKLIN ST, BOSTON, MA, 02110, US

(21) Appl. No.: 10/423,725

(22) Filed: Apr. 25, 2003

Related U.S. Application Data
(60) Provisional application No. 60/375,927, filed on Apr. 25, 2002.
(60) Provisional application No. 60/375,970, filed on Apr. 25, 2002.
(60) Provisional application No. 60/383,926, filed on May 28, 2002.
(60) Provisional application No. 60/393,959, filed on Jul. 5, 2002.
(60) Provisional application No. 60/446,432, filed on Feb. 10, 2003.

(51) Int. Cl. A61K 31/727; A61K 31/715; C12P 19/28; C12P 19/04

(52) U.S. Cl. 514/54; 514/56; 435/85; 435/101

(57) ABSTRACT

The invention features methods and products associated with non-invasive delivery of polysaccharide preparations.

This application claims priority to U.S. Provisional Patent Application Serial Nos. 60/375,927, filed Apr. 25, 2002; 60/375,970, filed Apr. 25, 2002; 60/383,926, filed May 28, 2002; 60/393,959, filed Jul. 23, 2002; and 60/446,432, filed Feb. 10, 2003, the entire contents of which are hereby incorporated by reference.

### TECHNICAL FIELD

The invention relates to methods and products associated with non-invasive delivery of polysaccharide preparations.

### BACKGROUND OF THE INVENTION

Recent advances in medicine have produced several alternative modes of drug delivery. Drugs which were previously only available in injectable forms, are now available in less invasive forms such as oral tablets or capsules, sustained release devices, and transdermal patches. Many of these advances, however, have occurred with protein based or small molecule drugs. Delivery of polysaccharides for therapeutic or prophylactic purposes is still associated with some problems.

### SUMMARY OF THE INVENTION

The invention is based, in part, on the discovery that polysaccharides such as heparin and low molecular weight heparin (LMWH) are amenable to non-invasive delivery at therapeutically effective levels. For example, sulfates can play an important role in determining the nature and activity of sulfonated polysaccharides. Sulfates can contribute, for example, to the ionic nature and functional interactions, e.g., anticoagulation, of heparin and LMWH. It was found that, although sulfates are generally labile to acidic conditions, sulfonated polysaccharides such as heparin and LWMH are stable in the acidic and enzymatic environment of the stomach. Thus, such polysaccharides can be used, e.g., for oral administration, which requires passage through the stomach prior to absorption by the intestinal membrane. In addition, it was found that based upon the chemical signatures of oligosaccharides, information regarding the number and identity of mono- or disaccharide building blocks can be determined, as can physiochemical properties of a polysaccharide including overall charge, charge density, molecular size, size to charge ratio and iduronic/glucuronic acid content. Thus, information obtained from the chemical signature can be used to obtain polysaccharides which are enhanced for non-invasive delivery routes, e.g., pulmonary, transdermal, and mucosal delivery.

Accordingly, in one aspect, the invention features a method for preparing a polysaccharide, e.g., a GAG, e.g., an HLGAG, for in vivo delivery, e.g., non-invasive delivery, e.g., transdermal, pulmonary or mucosal delivery. Examples of non-invasive delivery include pulmonary, transdermal, nasal, oral, sublingual, buccal, rectal or vaginal delivery. The method includes neutralizing a polysaccharide, to thereby prepare the polysaccharide for in vivo delivery. The method can further include reducing the mass of the polysaccharide.

In one embodiment, the method includes determining a chemical signature for the polysaccharide, and neutralizing the polysaccharide based upon its chemical signature. The method can further include reducing the mass of the polysaccharide based upon its chemical signature.

In one embodiment, the net negative or net positive charge of the polysaccharide is reduced by at least 10%, 20%, 30%, 40%, 50%, 60%, 70%, 80%, or 90%. In other embodiments, the polysaccharide is neutralized such that there is a net negative and net positive charge of 0. The polysaccharide can be neutralized, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lyase such as heparinase I, beparinase II, heparinase III, heparinase IV, heparanase, and functionally active fragments and variants thereof. Examples of chemicals which can be used include oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

In other embodiments, the polysaccharide is neutralized by contacting the polysaccharide with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In one embodiment, the polysaccharide is an HLGAG. Preferably, the HLGAG is unfractionated heparin or fractionated heparin (e.g., a low molecular weight heparin) or a synthetic pentasaccharide, e.g., Arixtra. Examples of LMWH include enoxaparin, dalteparin, reviparin, tinzaparin, nadroparin, certoparin, ardeparin, and parnaparin.

In one embodiment, the method further includes creating particles of the polysaccharide, e.g., particles having a mean geometric diameter of 1-500 microns, Preferably, particles having a mean geometric diameter of at least about 2, 3, 4, 5, 10, 15, 20, 25, 30, 35, 40, 45, 50, 100 microns are created.

In some embodiments, the method further includes using the polysaccharide as a carrier, e.g., by linking to the polysaccharide an active agent. The active agent can be, e.g., a nucleic acid, polypeptide, small molecule, a heterogeneous mixture, etc.

In embodiments, the method further includes formulating the polysaccharide with a delivery enhancer, e.g., a surfactant, an absorption enhancer, a polymer, etc.

In some embodiments, the polysaccharide is in a preparation of polysaccharides and the polydispersity of the preparation is less than 1.3, 1.2, 1.1 or 1 (and integers therebetween).

In another aspect, the invention features a method for preparing an HLGAG, e.g., heparin, for non-invasive in vivo delivery, e.g., transdermal, pulmonary or mucosal delivery. Examples of non-invasive delivery include pulmonary, transdermal, nasal, oral, sublingual, buccal, rectal or vaginal delivery. The method includes neutralizing an HLGAG, e.g., heparin, to thereby prepare the HLGAG for non-invasive in vivo delivery. The method can further include reducing the mass of the polysaccharide.

In some embodiments, the HLGAG is unfractionated or fractioned heparin (LMWH) or a synthetic pentasaccharide, e.g., Arixtra. Preferably, the HLGAG is a LMWH, e.g., enoxaparin, dalteparin, reviparin, tinzaparin, nadroparin, certoparin, ardeparin, or parnaparin. In one embodiment, the method includes determining a chemical signature for the heparin, and neutralizing the heparin based upon its chemical signature. The method can further include reducing the mass of the heparin based upon its chemical signature.

In one embodiment, the net negative or net positive charge of the heparin is reduced by at least 10%, 20%, 30%, 40%, 50%, 60%, 70%, 80%, or 90%. In other embodiments, the heparin is neutralized such that there is a net negative and net positive charge of 0. For example, the net charge of enoxaparin is about 19.23. Thus, in one embodiment, the heparin is a heparin-derived from enoxaparin, and the net charge of the heparin is less than about 19, 18, 17, 16, 15, 14, 13, 12, 11, 10, 9, 8, 7, 6, 5, 4, 3, 2, or 1. In another embodiment, the heparin is a heparin-derived from dalteparin and the net charge of the heparin is less than about 22, 21, 20, 19, 18, 17, 16, 15, 14, 13, 12, 11, 10, 9, 8, 7, 6, 5, 4, 3, 2, or 1. In another preferred embodiment, the heparin is a heparin derived from nadroparin and the net charge of the heparin is less than 27, 26, 25, 24, 23, 22, 21, 20, 19, 18, 17, 16, 15, 14, 13, 12, 11, 10, 9, 8, 7, 6, 5, 4, 3, 2, or 1. In yet another preferred embodiment, the heparin is a heparin-derived from reviparin and the heparin has a net charge of less than 25, 24, 23, 22, 21, 20, 19, 18, 17, 16, 15, 14, 13, 12, 11, 10, 9, 8, 7, 6, 5, 4, 3, 2, or 1. In another preferred embodiment, the heparin is a heparin derived from parnaparin and the net charge of the heparin is less than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1. In yet another preferred embodiment, the heparin is a heparin-derived from tinzaparin and the heparin has a net charge of less than 28, 27, 26, 25, 24, 23, 22, 21, 20, 19, 18, 17, 16, 15, 14, 13, 12, 11, 10, 9, 8, 7, 6, 5, 4, 3, 2, or 1. The heparin can be neutralized, e.g., by digesting the heparin with at least one agent, e.g., an agent selected based upon the chemical signature of the heparin. For example, the agent can be an enzyme (e.g., an enzyme which is capable of cleaving the heparin at known locations in the heparin based upon its chemical signature) or a chemical (e.g., a chemical capable of cleaving the heparin at known locations in the heparin based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

In other embodiments, the heparin is neutralized by contacting the heparin with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In one embodiment, the heparin, e.g., LMWH, can be neutralized such that at least one biological activity of the heparin is maintained. For example, one or more of the following activities of heparin can be maintained: anti-Xa activity, anti-IIa activity, FGF-2 binding activity, platelet factor 4 (PF4) binding activity or other measure of HIT propensity, protamine neutralization. In other embodiments, the heparin can be neutralized such that at least one biological activity, e.g., anti-Xa activity and/or anti-IIa activity, is at least partially maintained or enhanced, and at least one other biological activity, e.g., PF4 binding, is reduced. The presence or absence of one or more activities of heparin can be determined, e.g., based upon the chemical signature of the neutralized heparin. For example, the heparin, e.g., LMWH, can be analyzed for the presence (or absence) and quantity of one or more of the following components: I/GH_{NAc,6S}I/GH_{NS,3S,6S}, I/GH_{NS,6S}GH_{NS,3S,6S}, I/GH_{NAc,6S}GH_{NS,3S}, I/GH_{NS,6S}I/GH_{NS,3S}, I/GH_{NS,6S}I/GH_{NS,3S,6S}, I/GH_{NAc,6S}GH_{NS,3S}, I/GH_{NS,6S}I/GH_{NS,3S} or combinations thereof, as well as non-natural, e.g., modified, sugars. These signatures can be detected, e.g., by measuring their molecular weight, and sequencing, or by NMR, or the signature can be detected indirectly, e.g., by detecting their derivatives (e.g., ΔUH_{NAC,6S}GH_{NS,3S,6S}, ΔUH_{NS,6S}GH_{NS,3S,6S}, ΔUH_{NAc,6S}GH_{NS,3S}, ΔUH_{NS,6S}GH_{NS,3S}, ΔUH_{NS,6S}GH_{NS,3S,6S}. ΔUH_{NAC,6S}GH_{NS,3S}, ΔUH_{NS,6S}GH_{NS,3S} or combinations thereof, as well as non-natural, e.g., modified, sugars. In other embodiments, standard assays can be used to determine, e.g., protamine neutralization, anti-Xa or Ia activity (e.g., ACT), PF4 binding or other measure for HIT propensity and/or FGF-2 binding activity.

In one embodiment, the heparin being produced is derived from enoxaparin and the enoxaparin is neutralized and the mass reduced such that the heparin has a charge to mass ratio that is less than about 19.32/4200. In another embodiment, the heparin being produced is derived from nadroparin and the nadroparin is neutralized and the mass reduced such that the heparin has a charge to mass ratio that is less than about 27.6/6000. In yet other embodiments, the heparin being produced can be, for example, a neutralized dalteparin having a charge to mass ratio of less than about 23/5000, or a neutralized reviparin having a charge to mass ratio of less than about 25.3/5500. In another embodiment, the heparin being produced is derived from parnaparin and the parnaparin is neutralized and the mass reduced such that the heparin has a charge to mass ratio that is less than about 30.4/6610. In yet other embodiments, the heparin being produced can be, for example, a neutralized tinzaparin having a charge to mass ratio of less than about 28.06/6100.

In one embodiment, the method further includes creating particles of the heparin, e.g., particles having a mean geometric diameter of 1-500 microns, Preferably, particles having a mean geometric diameter of at least about 2, 3, 4, 5, 10, 15, 20, 25, 30, 35, 40, 45, 50, 100 microns are created.

In some embodiments, the method further includes using the heparin as a carrier, e.g., by linking the heparin to an active agent. The active agent can be, e.g., a nucleic acid, polypeptide, small molecule, a heterogeneous mixture, etc.

In some embodiments, the method further includes formulating the heparin with a delivery enhancer, e.g., a surfactant, an absorption enhancer, a polymer, etc.

In some embodiments, the heparin is in a preparation of heparin and the polydispersity of the heparin preparation is less than 1.3, 1.2, 1.1 or 1 (and integers therebetween).

In another aspect, the invention features a method for preparing a polysaccharide, e.g., a GAG, an HLGAG, for non-invasive in vivo delivery. Examples of non-invasive delivery include pulmonary, transdermal, nasal, oral, sublingual, buccal, rectal or vaginal delivery. The method includes: providing a polysaccharide; determining a chemical signature for the polysaccharide; and reducing the mass of the polysaccharide based upon its chemical signature, to thereby prepare the polysaccharide for non-invasive in vivo delivery. The method can further include neutralizing the polysaccharide.

In a preferred embodiment, the mass of the polysaccharide can be reduced such that at least one or more activity of the polysaccharide is maintained. In other preferred embodiments, the mass of the polysaccharide can be reduced such that at least one activity of the polysaccharide is at least partially maintained or enhanced and at least one other activity of the polysaccharide is reduced.

In one embodiment, the mass of the polysaccharide is reduced by at least 5%, 10%, 15%, 20%, 25%, 30%, 35%, 40%, 45%, 50%, 55%, 60%, 65%, 70%, 75%, 80%, 85%, or 90% (and integers there between) from the mass of the provided polysaccharide. In other embodiments, the mass of the polysaccharide is reduced by at least 5, 10, 15, 20, 25, 30, 35, 40, 50, 60, 70, 80, 90, 100, 150, 200, 250, 300, 350, 500, 1000, 1500, 2000, 2200, 2500, 3000 Da or more from the mass of the provided polysaccharide.

In one embodiment, the mass of the provided polysaccharide can be reduced, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

In some embodiments, when the charge of the polysaccharide is neutralized, the net negative or net positive charge of the polysaccharide can be reduced by at least 10%, 20%, 30%, 40%, 50%, 60%, 70%, 80%, or 90%. In other embodiments, when the charge of the polysaccharide is neutralized, it can be neutralized such that there is a net negative and net positive charge of 0. The polysaccharide can be neutralized, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

In other embodiments, when the charge of the polysaccharide is neutralized, it can be neutralized by contacting the polysaccharide with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In one embodiment, the polysaccharide is an HLGAG. Preferably, the HLGAG is unfractionated heparin or fractionated heparin (e.g., LMWH) or a synthetic pentasaccharide, e.g., Arixtra. Examples of LMWH include enoxaparin, dalteparin, reviparin, tinzaparin, nadroparin, certoparin, ardeparin, and parnaparin.

In one embodiment, the method further includes creating particles of the polysaccharide, e.g., particles having a mean geometric diameter of 1-500 microns, Preferably, particles having a mean geometric diameter of at least about 2, 3, 4, 5, 10, 15, 20, 25, 30, 35, 40, 45, 50, 100 microns are created.

In some embodiments, the method further includes using the polysaccharide as a carrier, e.g., by linking to the polysaccharide an active agent. The active agent can be, e.g., a nucleic acid, polypeptide, small molecule, a heterogeneous mixture, etc.

In some embodiments, the method further includes formulating the polysaccharide with a delivery enhancer, e.g., a surfactant, an absorption enhancer, a polymer, etc.

In some embodiments, the polysaccharide is in a preparation of polysaccharides and the polydispersity of the heparin preparation is less than 1.3, 1.2, 1.1, or 1 (and integers therebetween).

In another aspect, the invention features a method for preparing an HLGAG, e.g., heparin, for non-invasive in vivo delivery. Examples of non-invasive delivery include pulmonary, transdermal, nasal, oral, sublingual, buccal, rectal or vaginal delivery. The method includes: providing an HLGAG; determining a chemical signature for the HLGAG; and reducing the mass of the HLGAG based upon its chemical signature, to thereby prepare the HLGAG for non-invasive in vivo delivery. The method can further include neutralizing the HLGAG.

In a preferred embodiment, the HLGAG is a heparin, e.g., an unfractionated or fractionated heparin or a synthetic pentasaccharide, e.g., Arixtra. Preferably, the heparin is a LMWH such as enoxaparin, dalteparin, reviparin, tinzaparin, nadroparin, certoparin, ardeparin, or parnaparin.

In a preferred embodiment, the mass of the heparin can be reduced such that at least one or more activity of the heparin is maintained. In other preferred embodiments, the mass of the heparin can be reduced such that at least one activity of the heparin is at least partially maintained or enhanced and at least one other activity of the heparin is reduced. The presence or absence of one or more activities of heparin can be determined, e.g., based upon the chemical signature of the neutralized heparin. For example, the heparin, e.g., LMWH, can be analyzed for the presence (or absence) of one or more of the following components: I/GH_{NAC,6S}I/GH_{NS,3S,6S}, I/GH_{NS,6S}GH_{NS,3S,6S}, I/GH_{NAc,6S}GH_{NS,3S}, I/GH_{NS,6S}I/GH_{NS,3S}, I/GH_{NS,6S}I/GH_{NS,3S,6S}, I/GH_{NAc,6S}GH_{NS,3S}, I/GH_{NS,6S}I/GH_{NS,3S} or combinations thereof, as well as non-natural, e.g., modified, sugars. These signatures can be detected, e.g., by measuring their molecular weight, and sequencing, or by NMR, or the signature can be detected indirectly, e.g., by detecting their derivatives (e.g., ΔUH_{NAc,6S}GH_{NS,3S,6S}, ΔUH_{NS,6S}GH_{NS,3S,6S}, ΔUH_{NAc,6S}GH_{NS,3S}, ΔUH_{NS,6S}GH_{NS,3S}, ΔUH_{NS,6S}GH_{NS,3S,6S}, ΔUH_{NAc,6S}GH_{NS,3S}, ΔUH_{NS,6S}GH_{NS,3S} or combinations thereof, as well as non-natural, e.g., modified, sugars. In other embodiments, standard assays can be used to determine, e.g., protamine neutralization, anti-Xa or IIa activity (e.g., ACT), PF4 binding (or other measure of HIT propensity) and/or FGF-2 binding activity.

In one embodiment, the mass of the heparin can be reduced by at least 5%, 10%, 15%, 20%, 25%, 30%, 35%, 40%, 45%, 50%, 55%, 60%, 65%, 70%, 75%, 80%, 85%, or 90% (and integers there between) from the mass of the provided heparin. In other embodiments, the mass of the heparin can be reduced by at least 5, 10, 15, 20, 25, 30, 35, 40, 50, 60, 70, 80, 90, 100, 150, 200, 250, 300, 350, 500, 1000, 1500, 2000, 2200, 2500, 3000 Da or more from the mass of the provided heparin.

In one embodiment, the mass of the provided polysaccharide can be reduced, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

In one embodiment, the heparin is a heparin derived from enoxaparin and the mass of enoxaparin is reduced such that the mass of the heparin is about 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000, 1900, 1800, 1700 Da or less. In another embodiment, the heparin is derived from nadroparin and the mass of nadroparin is reduced such that the mass of the heparin is about 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In other embodiments, the heparin is derived from dalteparin and the mass of dalteparin is reduced such that the mass of the heparin is about 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In yet another embodiment, the heparin is derived from reviparin and the mass of the reviparin is reduced such that the mass of the heparin is about 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In another embodiment, the heparin is derived from parnaparin and the mass of parnaparin is reduced such that the mass of the heparin is about 6,500, 6,400, 6,300, 6,200, 6,100, 6,000, 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In yet another embodiment, the heparin is derived from tinzaparin and the mass of tinzaparin is reduced such that the mass of the heparin is about 6,00, 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less.

In some embodiments, when the charge of the heparin is neutralized, the net negative or net positive charge of the heparin can be reduced by at least 10%, 20%, 30%, 40%, 50%, 60%, 70%, 80%, or 90%. In other embodiments, when the charge of the heparin is neutralized, it can be neutralized such that there is a net negative and net positive charge of 0. For example, the net charge of enoxaparin is about 19.23. Thus, in one embodiment, the heparin is a heparin-derived from enoxaparin, and the net charge of the heparin is less than about 19, 18, 17, 16, 15, 14, 13, 12, 11, 10, 9, 8, 7, 6, 5, 4, 3, 2, or 1. In another embodiment, the heparin is a heparin-derived from nadroparin and the net charge is less than 27, 26, 25, 24, 23, 22, 21, 20, 19, 18, 17, 16, 15, 14, 13, 12, 11, 10, 9, 8, 7, 6, 5, 4, 3, 2, or 1. In another embodiment, the heparin is a heparin-derived from dalteparin and the net charge of the heparin is less than about 22, 21, 20, 19, 18, 17, 16, 15, 14, 13, 12, 11, 10, 9, 8, 7, 6, 5, 4, 3, 2, or 1. In yet another embodiment, the heparin is a heparin-derived from reviparin and the net charge of the heparin is less than 25, 24, 23, 22, 21, 20, 19, 18, 17, 16, 15, 14, 13, 12, 11, 10, 9, 8, 7, 6, 5, 4, 3, 2, or 1. In another preferred embodiment, the heparin is a heparin derived from parnaparin and the net charge of the heparin is less than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1. In yet another preferred embodiment, the heparin is a heparin-derived from tinzaparin and the heparin has a net charge of less than 28, 27, 26, 25, 24, 23, 22, 21, 20, 19, 18, 17, 16, 15, 14, 13, 12, 11, 10, 9, 8, 7, 6, 5, 4, 3, 2, or 1. The heparin can be neutralized, e.g., by digesting the heparin with at least one agent, e.g., an agent selected based upon the chemical signature of the heparin. For example, the agent can be an enzyme (e.g., an enzyme which is capable of cleaving the heparin at known locations in the heparin based upon its chemical signature) or a chemical (e.g., a chemical capable of cleaving the heparin at known locations in the heparin based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

In other embodiments, when the charge of the heparin is neutralized, it can be neutralized by contacting the heparin with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In one embodiment, the heparin being produced is derived from enoxaparin and the enoxaparin is neutralized and the mass reduced such that the heparin has a charge to mass ratio that is less than about 19.32/4200. In another embodiment, the heparin being produced is derived from nadroparin and the nadroparin is neutralized and the mass reduced such that the heparin has a charge to mass ratio that is less than about 27.6/6000. In yet other embodiments, the heparin being produced can be, for example, a neutralized dalteparin having a charge to mass ratio of less than about 23/5000, or a neutralized reviparin having a charge to mass ratio of less than about 25.3/5500. In another embodiment, the heparin being produced is derived from parnaparin and the parnaparin is neutralized and the mass reduced such that the heparin has a charge to mass ratio that is less than about 30.4/6610. In yet other embodiments, the heparin being produced can be, for example, a neutralized tinzaparin having a charge to mass ratio of less than about 28.06/6100.

In one embodiment, the method further includes creating particles of the heparin, e.g., particles having a mean geometric diameter of 1-500 microns, Preferably, particles having a mean geometric diameter of at least about 2, 3, 4, 5, 10, 15, 20, 25, 30, 35, 40, 45, 50, 100 microns are created.

In some embodiments, the method further includes using the produced heparin as a carrier, e.g., by linking the heparin to an active agent. The active agent can be, e.g., a nucleic acid, polypeptide, small molecule, a heterogeneous mixture, etc.

In some embodiments, the method further includes formulating the produced heparin with a delivery enhancer, e.g., a surfactant, an absorption enhancer, a polymer, etc.

In some embodiments, the heparin is in a preparation of heparin and the preparation has a polydispersity of less than 1.3, 1.2, 1.1, or 1 (and integers therebetween).

In another aspect, the invention features a polysaccharide composition for non-invasive in vivo delivery made by a method described above.

In yet another aspect, the invention features a composition for non-invasive delivery, e.g., transdermal, pulmonary or mucosal delivery, wherein the composition comprises a therapeutically effective amount of a sulfonated polysaccharide, e.g., a sulfonated HLGAG.

In preferred embodiments, the sulfonated HLGAG is a heparin, e.g., a fractionated or unfractioned heparin or a synthetic pentasaccharide, e.g., Arixtra. Preferably, the heparin is a LMWH such as enoxaparin, dalteparin, reviparin, tinzaparin, nadroparin, certoparin, ardeparin, or parnaparin.

In a preferred embodiment, the composition is for mucosal delivery (e.g., oral, buccal, sublingual, rectal, or vaginal delivery).

In one embodiment, the composition includes particles of the polysaccharide, e.g., particles having a mean geometric diameter of 1-500 microns. Preferably, particles having a mean geometric diameter of at least about 2, 3, 4, 5, 10, 15, 20, 25, 30, 35, 40, 45, 50, 100 microns are created.

In some embodiments, the sulfonated polysaccharide is used as a carrier, e.g., the composition further includes an active agent, e.g., the sulfonated polysaccharide is linked to the active agent. The active agent can be, e.g., a nucleic acid, polypeptide, small molecule, a heterogeneous mixture, etc.

In some embodiments, the composition further includes a delivery enhancer, e.g., a surfactant, an absorption enhancer, a polymer, etc.

In yet another aspect, the invention features a composition for pulmonary delivery, wherein the composition comprises a therapeutically effective amount of an HLGAG, e.g., a synthetic HLGAG, e.g., a synthetic pentasaccharide, e.g., Arixtra. In another embodiment, the HLGAG is a LMWH, e.g., a LMWH selected from the group consisting of: enoxaparin, dalteparin, reviparin, tinzaparin, nadroparin, certoparin, ardeparin, and parnaparin. Preferably, the composition is in a device which delivers a therapeutically effective unit dose of the HLGAG, e.g., the synthetic HLGAG, e.g., the synthetic pentasaccharide, e.g., Arixtra, to provide a preselected therapeutic effect, e.g., anti-Xa and or anti-IIa activity.

In one embodiment, the therapeutically effective unit dose of the HLGAG, e.g., the synthetic HLGAG, is about 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg or doses therebetween. In another embodiment, the therapeutically effective unit dose of the HLGAG, e.g., the synthetic HLGAG is about 3 mg, 4 mg, 5 mg, 6, mg, 7 mg, 8 mg, 16 mg, 48 mg, 80 mg, 120 mg or doses therebetween. In a preferred embodiment, the synthetic HLGAG is Arixtra. In other embodiments, the synthetic HLGAG is one or more of the compounds provided in FIG. 9 and derivatives thereof.

In another embodiment, the HLGAG is a LMWH (e.g., a LMWH selected from the group consisting of: enoxaparin, dalteparin, reviparin, tinzaparin, nadroparin, certoparin, ardeparin, and parnaparin) and the therapeutically effective unit dose of the LMWH is about 2 mg/kg, 2.2 mg/kg, 2.5 mg/kg, 2.7 mg/kg, 3.0 mg/kg, 3.2 mg/kg, 3.5 mg/kg, 3.7 mg/kg, 4.0 mg/kg, 4.5 mg/kg, 5 mg/kg or doses therebetween. In another embodiment, the therapeutically effective unit dose of the LMWH is about 160 mg, 180 mg, 200 mg, 220, mg, 240 mg, 260 mg, 280 mg, 300 mg, 350 mg, 400 mg or doses therebetween. In a preferred embodiment, the LMWH is ardeparin.

In another embodiment, the therapeutically effective unit doses of the HLGAG, e.g., the synthetic HLGAG or LMWH, is amount effective to produce a peak plasma concentration of the HLGAG, e.g., the synthetic HLGAG, within about 5 minutes to about 5 hours, 10 minutes to about 3 hours, 30 minutes to about 2 hours, after delivery.

In one embodiment, the HLGAG, e.g., the synthetic HLGAG or LMWH is in the form of a solid, e.g., a dry particle. In one embodiment, the synthetic HLGAG or LMWH is in the form of a dry particle and the particle has a mean geometric diameter of 1-500 microns (e.g., 53-75 microns). Preferably, particles having a mean geometric diameter of at least about 2, 3, 4, 5, 10, 15, 20, 25, 30, 35, 40, 45, 50, 100 microns are created. In other embodiments, the HLGAG, e.g., the synthetic HLGAG, is in the form of a liquid.

In one embodiment, the HLGAG is Arixtra and the therapeutically effective unit dose is about 0.01 mg/kg, 0.03 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg or doses therebetween. In another embodiment, the therapeutically effective unit dose of the Arixtra is about 3 mg, 4 mg, 5 mg, 6 mg, 7 mg, 8 mg, 16 mg, 48 mg, 80 mg, 120 mg or doses therebetween.

In another embodiment, the HLGAG is Arixtra and the therapeutically effective unit doses is amount effective to produce a peak plasma concentration of the Arixtra within about 5 minutes to about 5 hours, 10 minutes to about 3 hours, 30 minutes to about 2 hours, after delivery.

In some embodiments, the composition further includes a pulmonary delivery enhancer, e.g., a surfactant.

In a preferred embodiment, the device is a pressurized container or dispenser, e.g., a pressurized container or dispenser which contains a suitable propellant and/or nebulizer. In one embodiment, the pressurized container or dispenser is a pressurized pack. In another embodiment, the pressurized container or dispenser is a nebulizer.

In yet another aspect, the invention features a method for preparing an HLGAG, e.g., a LMWH or a synthetic HLGAG (e.g., a synthetic pentasaccharide, e.g., Arixtra) for pulmonary delivery which provides a preselected therapeutic effect, e.g., anti-Xa and/or anti-IIa activity. The method includes providing the HLGAG in a device that delivers a therapeutically effective unit dose of the HLGAG by pulmonary administration to provide the desired effect.

In one embodiment, the unit dose is at least 2, preferably 3, more preferably 4 or 5 times greater than the dose used to provide the preselected result by subcutaneous or intravenous administration of the HLGAG, e.g., the LMWH or the synthetic HLGAG (e.g., the synthetic pentasaccharide).

In one embodiment, the therapeutically effective unit dose of the synthetic HLGAG, is about 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg or doses therebetween. In another embodiment, the therapeutically effective unit dose of the synthetic HLGAG is about 8 mg, 16 mg, 48 mg, 80 mg, 120 mg or doses therebetween. In a preferred embodiment, the synthetic HLGAG is Arixtra and derivatives thereof. In other embodiments, the synthetic HLGAG is one or more of the compounds provided in FIG. 9 and derivatives thereof.

In another embodiment, the HLGAG is a LMWH (e.g., a LMWH selected from the group consisting of: enoxaparin, dalteparin, reviparin, tinzaparin, nadroparin, certoparin, ardeparin, and parnaparin) and the therapeutically effective unit dose of the LMWH is about 2 mg/kg, 2.2 mg/kg, 2.5 mg/kg, 2.7 mg/kg, 3.0 mg/kg, 3.2 mg/kg, 3.5 mg/kg, 3.7 mg/kg, 4.0 mg/kg, 4.5 mg/kg, 5 mg/kg or doses therebetween. In another embodiment, the therapeutically effective unit dose of the LMWH is about 160 mg, 180 mg, 200 mg, 220, mg, 240 mg, 260 mg, 280 mg, 300 mg, 350 mg, 400 mg or doses therebetween. In a preferred embodiment, the LMWH is ardeparin.

In another embodiment, the therapeutically effective unit doses of the HLGAG, e.g., the LMWH or the synthetic HLGAG, is amount effective to produce a peak plasma concentration of the HLGAG, e.g., the LMWH or the synthetic HLGAG, within about 5 minutes to about 5 hours, 10 minutes to about 3 hours, 30 minutes to about 2 hours, after delivery.

In one embodiment, the HLGAG, e.g., the LMWH or the synthetic HLGAG is in the form of a solid, e.g., a dry particle. In one embodiment, the synthetic HLGAG or LMWH is in the form of a dry particle and the particle has a mean geometric diameter of 1-500 microns (e.g., 53-75 microns). Preferably, particles having a mean geometric diameter of at least about 2, 3, 4, 5, 10, 15, 20, 25, 30, 35, 40, 45, 50, 100 microns are created. In other embodiments, the HLGAG, e.g., the LMWH or the synthetic HLGAG, is in the form of a liquid.

In one embodiment, the HLGAG is Arixtra and the therapeutically effective unit dose is about 0.01 mg/kg, 0.03 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg or doses therebetween. In another embodiment, the therapeutically effective unit dose of the Arixtra is about 3 mg, 4 mg, 5 mg, 6 mg, 7 mg, 8 mg, 16 mg, 48 mg, 80 mg, 120 mg or doses therebetween.

In another embodiment, the HLGAG is Arixtra and the therapeutically effective unit doses is amount effective to produce a peak plasma concentration of the Arixtra within about 5 minutes to about 5 hours, 10 minutes to about 3 hours, 30 minutes to about 2 hours, after delivery.

In some embodiments, the method further includes providing a pulmonary delivery enhancer, e.g., a surfactant, and/or a pharmaceutically acceptable carrier.

In some embodiments, the method further includes providing the composition in a device for pulmonary delivery. The device can be a pressurized container or dispenser, e.g., a pressurized container or dispenser which contains a suitable propellant and/or nebulizer. In one embodiment, the pressurized container or dispenser is a pressurized pack. In another embodiment, the pressurized container or dispenser is a nebulizer.

In other aspects, the invention features a composition for non-invasive delivery, e.g., transdermal, pulmonary or mucosal delivery, of a heparin, e.g., an unfractionated or fractionated heparin (LMWH) or a synthetic pentasaccharide, e.g., Arixtra, wherein the heparin has a net negative charge which is less than a reference net charge for the heparin. In some embodiments, the heparin further has a mass which is less than a reference mass for heparin.

In one embodiment, the composition is for oral delivery.

In preferred embodiments, the heparin is a LMWH. Preferably, the LMWH is a charge neutralized enoxaparin, dalteparin, reviparin, tinzaparin, nadroparin, certoparin, ardeparin, or parnaparin.

In a preferred embodiment, the heparin has a negative net charge of less than the reference net charge of enoxaparin. Thus, in one embodiment, the heparin is a heparin-derived from enoxaparin, and the net charge of the heparin is less than about 19, 18, 17, 16, 15, 14, 13, 12, 11, 10, 9, 8, 7, 6, 5, 4, 3, 2, or 1. In another embodiment, the heparin is a heparin-derived from nadroparin and the net charge is less than 27, 26, 25, 24, 23, 22, 21, 20, 19, 18, 17, 16, 15, 14, 13, 12, 11, 10, 9, 8, 7, 6, 5, 4, 3, 2, or 1. In another embodiment, the heparin is a heparin-derived from dalteparin and the net charge of the heparin is less than about 22, 21, 20, 19, 18, 17, 16, 15, 14, 13, 12, 11, 10, 9, 8, 7, 6, 5, 4, 3, 2, or 1. In yet another embodiment, the heparin is a heparin-derived from reviparin and the net charge of the heparin is less than 25, 24, 23, 22, 21, 20, 19, 18, 17, 16, 15, 14, 13, 12, 11, 10, 9, 8, 7, 6, 5, 4, 3, 2, or 1. In another preferred embodiment, the heparin is a heparin derived from parnaparin and the net charge of the heparin is less than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1. In yet another preferred embodiment, the heparin is a heparin-derived from tinzaparin and the heparin has a net charge of less than 28, 27, 26, 25, 24, 23, 22, 21, 20, 19, 18, 17, 16, 15, 14, 13, 12, 11, 10, 9, 8, 7, 6, 5, 4, 3, 2, or 1. Preferably, the net charge of the LMWH is at least 10%, 20%, 30%, 40%, 50%, 60%, 70%, 80%, 90%, 100% less than the net charge of enoxaparin.

In one embodiment, when the heparin further has a mass less than a reference mass, and the reference mass is the mass of enoxaparin. Preferably, the mass of the heparin is at least 5, 10, 15, 20, 25, 30, 35, 40, 50, 60, 70, 80, 90, 100, 150, 200, 250, 300, 350, 500, 1000, 1500, 2000, 2500 Da less than the mass of enoxaparin. In another embodiment, the reference mass is the mass of nadroparin. Preferably, the mass of the heparin is at least 5, 10, 15, 20, 25, 30, 35, 40, 50, 60, 70, 80, 90, 100, 150, 200, 250, 300, 350, 500, 1000, 1500, 2000, 2500 Da less than the mass of nadroparin. In other preferred embodiments, the reference mass is the mass of dalteparin and the mass of the heparin is at least 5, 10, 15, 20, 25, 30, 35, 40, 50, 60, 70, 80, 90, 100, 150, 200, 250, 300, 350, 500, 1000, 1500, 2000, 2500 Da less than the mass of dalteparin. In yet other embodiments, the reference mass is the mass of reviparin and the mass of the heparin is at least 5, 10, 15, 20, 25, 30, 35, 40, 50, 60, 70, 80, 90, 100, 150, 200, 250, 300, 350, 500, 1000, 1500, 2000, 2500 Da less than the mass of reviparin. In other preferred embodiments, the reference mass is the mass of parnaparin and the mass of the heparin is at least 5, 10, 15, 20, 25, 30, 35, 40, 50, 60, 70, 80, 90, 100, 150, 200, 250, 300, 350, 500, 1000, 1500, 2000, 2500 Da less than the mass of parnaparin. In yet other embodiments, the reference mass is the mass of tinzaparin and the mass of the heparin is at least 5, 10, 15, 20, 25, 30, 35, 40, 50, 60, 70, 80, 90, 100, 150, 200, 250, 300, 350, 500, 1000, 1500, 2000, 2500 Da less than the mass of tinzaparin.

In a some embodiments, the heparin is a LMWH and the mass of the LMWH is about 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700 Da or less.

In one embodiment, the heparin is derived from enoxaparin and the enoxaparin is neutralized and the mass reduced such that the heparin has a charge to mass ratio that is less than about 19.32/4200. In another embodiment, the heparin is derived from nadroparin and the nadroparin is neutralized and the mass reduced such that the heparin has a charge to mass ratio that is less than about 27.6/6000. In yet other embodiments, the heparin can be, for example, a neutralized dalteparin having a charge to mass ratio of less than about 23/5000, or a neutralized reviparin having a charge to mass ratio of less than about 25.3/5500. In another embodiment, the heparin is derived from parnaparin and the parnaparin is neutralized and the mass reduced such that the heparin has a charge to mass ratio that is less than about 30.4/6610. In yet other embodiments, the heparin can be, for example, a neutralized tinzaparin having a charge to mass ratio of less than about 28.06/6100.

In one embodiment, the heparin is in a dry particle formulation, e.g., a dry particle having a mean geometric diameter of at least 5, 10, 15, 20, 25, 30, 40, 50, 60, 70, 80, 90, 100, 120, 140, 160, 180, 200, 250, 300, 350, 400, 450 or 500 microns (or integers there between).

In another embodiment, the heparin is in an aqueous formulation.

In some embodiments, the composition further includes a charge neutralization agent. The charge neutralization agent can be, e.g., a counter ion, e.g., a mono- or divalent ion (barium, calcium, sodium, potassium, lithium, ammonium, magnesium, zinc), a transition metal (e.g., iron, nickel, copper), other charge neutralizing agents (e.g., spermine, spermidine, low molecular weight protamine, basic peptides).

In one embodiment, the heparin can be a carrier and the composition can further include an active agent, e.g., the active agent and the heparin are distinct. In other embodiments the heparin is the active agent.

In one embodiment, the composition further includes a delivery enhancer, e.g., a surfactant, an absorption enhancer, a polymer, etc.

In some embodiments, the heparin is in a preparation of heparin and the polydispersity of the preparation is less than 1.3, 1.2, 1.1, or 1 (and integers therebetween).

In other aspects, the invention features a composition for mucosal delivery comprising M405.

In a preferred embodiment, the composition has a polydispersity of less than 1.3, 1.2, 1.1. or 1 (and integers therebetween).

In another aspect, the invention features a composition for mucosal delivery comprising M108.

In a preferred embodiment, the composition has a polydispersity of less than 1.3, 1.2, 1.1. or 1 (and integers therebetween).

In another aspect, the invention features a composition for mucosal delivery comprising M115.

In a preferred embodiment, the composition has a polydispersity of less than 1.3, 1.2, 1.1. or 1 (and integers therebetween).

In another aspect, the invention features a composition for mucosal delivery comprising M411.

In a preferred embodiment, the composition has a polydispersity of less than 1.3, 1.2, 1.1. or 1 (and integers therebetween).

In another aspect, the invention features a composition for mucosal delivery comprising M118.

In a preferred embodiment, the composition has a polydispersity of less than 1.3, 1.2, 1.1. or 1 (and integers therebetween).

In another aspect, the invention features a composition for mucosal delivery comprising M312.

In a preferred embodiment, the composition has a polydispersity of less than 1.3, 1.2, 1.1. or 1 (and integers therebetween).

In another aspect, the invention features a method for delivering a sulfonated polysaccharide to a subject. The method includes orally administering to a subject a sulfonated polysaccharide (e.g., an HLGAG, e.g., a heparin) in a therapeutically effective amount, to thereby deliver the polysaccharide to the subject.

In a preferred embodiment, the sulfonated polysaccharide is heparin, e.g., unfractionated or fractioned heparin or a synthetic pentasaccharide, e.g., Arixtra. Preferably, the fractioned heparin is a LMWH. Examples of such LMWH include those LMWH described herein, e.g., charge neutralized and/or mass reduced enoxaparin, dalteparin, reviparin, tinzaparin, nadroparin, certoparin, ardeparin, and parnaparin, as well as M312, M118, M405, M108, M115, and M411.

In one embodiment, the polysaccharide is in the form of a solid. In other embodiments, the polysaccharide is in aqueous form.

The polysaccharide can also be included in a composition, e.g., a composition which includes a pharmaceutically acceptable carrier. The pharmaceutical composition can further include an active agent, e.g., an active agent distinct from the polysaccharide (e.g., the polysaccharide is a carrier), a delivery enhancer, etc.

In a preferred embodiment, the composition has a polydispersity of less than 1.3, 1.2, 1.1. or 1 (and integers therebetween).

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In another aspect, the invention features a method for in vivo non-invasive delivery (e.g., transdermal, pulmonary or mucosal delivery) of a polysaccharide to a subject. The method includes administering to a subject a therapeutically effective amount of a composition made by a method described herein, to thereby deliver the polysaccharide to the subject.

In preferred embodiments, the polysaccharide is a polysaccharide described herein.

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In another aspect, the invention features, a method for oral delivery of heparin, e.g., LMWH, to a subject. The method includes orally administering therapeutically effective amount of a heparin, e.g., LMWH, to a subject, wherein the heparin, e.g., LMWH, has a net negative charge which is less than a reference net charge for the heparin, to thereby deliver the heparin to the subject.

In preferred embodiments, the heparin is a synthetic pentasaccharide, e.g., Arixtra, or a LMWH. Examples of such LMWH include those LMWH described herein, e.g., charge neutralized and/or mass reduced enoxaparin, dalteparin, reviparin, tinzaparin, nadroparin, certoparin, ardeparin, and parnaparin, as well as M312, M118, M405, M108, M115, and M411.

In a preferred embodiment, the LMWH has a net charge that is less than the net charge of enoxaparin, e.g., the net charge of the LMWH is at least 10%, 20%, 30%, 40%, 50%, 60%, 70%, 80%, 90%, 100% less than the net charge of enoxaparin. For example, the net charge of enoxaparin is about 19.32. Thus, in one embodiment, the heparin has a net charge of less than about 19, 18, 17, 16, 15, 14, 13, 12, 11, 10, 9, 8, 7, 6, 5, 4, 3, 2, or 1. In another embodiment, the heparin is a heparin derived from nadroparin and the net charge of the heparin is less than 27, 26, 25, 24, 23, 22, 21, 20, 19, 18, 17, 16, 15, 14, 13, 12, 11, 10, 9, 8, 7, 6, 5, 4, 3, 2, or 1. In another embodiment, the heparin is a heparin-derived from dalteparin and the net charge of the heparin is less than about 22, 21, 20, 19, 18, 17, 16, 15, 14, 13, 12, 11, 10, 9, 8, 7, 6, 5, 4, 3, 2, or 1. In yet another embodiment, the heparin is a heparin derived from reviparin and the net charge of the heparin is less than 25, 24, 23, 22, 21, 20, 19, 18, 17, 16, 15, 14, 13, 12, 11, 10, 9, 8, 7, 6, 5, 4, 3, 2, or 1. In another preferred embodiment, the heparin is a heparin derived from parnaparin and the net charge of the heparin is less than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1. In yet another preferred embodiment, the heparin is a heparin-derived from tinzaparin and the heparin has a net charge of less than 28, 27, 26, 25, 24, 23, 22, 21, 20, 19, 18, 17, 16, 15, 14, 13, 12, 11, 10, 9, 8, 7, 6, 5, 4, 3, 2, or 1.

In other embodiments, the heparin further has a mass which less than a reference mass for the heparin. For example, the heparin is a LMWH and the LMWH has a mass that is less than the mass of enoxaparin. Preferably, the mass of the LMWH is less than at least 5, 10, 15, 20, 25, 30, 35, 40, 50, 60, 70, 80, 90, 100, 150, 200, 250, 300, 350, 500, 1000, 1500, 2000, 2500 Da than the mass of enoxaparin. In other preferred embodiments, the mass of the LMWH is about 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700 Da or less.

In one embodiment, the heparin is a heparin derived from enoxaparin and the mass of enoxaparin is reduced such that the mass of the heparin is about 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000, 1900, 1800, 1700 Da or less. In another embodiment, the heparin is derived from nadroparin and the mass of nadroparin is reduced such that the mass of the heparin is about 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In other embodiments, the heparin is derived from dalteparin and the mass of dalteparin is reduced such that the mass of the heparin is about 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In yet another embodiment, the heparin is derived from reviparin and the mass of the reviparin is reduced such that the mass of the heparin is about 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In another embodiment, the heparin is derived from parnaparin and the mass of parnaparin is reduced such that the mass of the heparin is about 6,500, 6,400, 6,300, 6,200, 6,100, 6,000, 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In yet another embodiment, the heparin is derived from tinzaparin and the mass of tinzaparin is reduced such that the mass of the heparin is about 6,00, 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less.

In one embodiment, the heparin is derived from enoxaparin and the enoxaparin is neutralized and the mass reduced such that the heparin has a charge to mass ratio that is less than about 19.32/4200. In another embodiment, the heparin is derived from nadroparin and the nadroparin is neutralized and the mass reduced such that the heparin has a charge to mass ratio that is less than about 27.6/6000. In yet other embodiments, the heparin can be, for example, a neutralized dalteparin having a charge to mass ratio of less than about 23/5000, or a neutralized reviparin having a charge to mass ratio of less than about 25.3/5500. In another embodiment, the heparin is derived from parnaparin and the parnaparin is neutralized and the mass reduced such that the heparin has a charge to mass ratio that is less than about 30.4/6610. In yet other embodiments, the heparin can be, for example, a neutralized tinzaparin having a charge to mass ratio of less than about 28.06/6100.

In a preferred embodiment, the composition has a polydispersity of less than 1.3, 1.2, 1.1. or 1 (and integers therebetween).

In one embodiment, the therapeutically effective amount of the LMWH is about 2 mg/kg, 3 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg.

In another embodiment, the LMWH has an absorption rate in the gastrointestinal tract of about 0.2 IU/ml, 0.25 IU/ml, 0.3 IU/ml, 0.35 IU/ml, 0.4 IU/ml or more over a period of about 1-5 hours, 2-4 hours.

In a preferred embodiment, at least 5%, 10%, 15%, 20% or more of the LMWH is delivered to the intestinal mucosa. In another embodiment, the LMWH is delivered to the intestinal mucosa in an amount effective to produce a peak plasma concentration of the LWMH within 2, 3, 4, 5, 6, 7 hours after delivery. In yet another embodiment, at least 5%, 10%, 15%, 20%, 25%, 30%, 40% or more of the LMWH is detectable in the blood within about 1-10, 2 to 7, 3 to 5 hours after delivery. In preferred embodiments, the bioavailability of the LMWH is at least about 15%, 20%, 25%, 30%, 35%, 40%, 50% or more.

In one embodiment, the heparin is in the form of a solid. In other embodiments, the heparin is in aqueous form.

The heparin can also be included in a composition, e.g., a composition which includes a pharmaceutically acceptable carrier. The pharmaceutical composition can further include an active agent, e.g., an active agent distinct from the heparin (e.g., the heparin is a carrier), a delivery enhancer, etc.

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In another aspect, the invention features a method for delivering M405 to a subject which includes orally administering to a subject a composition comprising M405 in an effective amount to deliver a therapeutic dose of M405 to the subject, thereby delivering M405 to the subject.

In one embodiment, the therapeutically effective amount of the M405 is about 1 mg/kg, 2 mg/kg, 3 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg.

In another embodiment, the M405 has an absorption rate in the gastrointestinal tract of about 0.2 IU/ml, 0.25 IU/ml, 0.3 IU/ml, 0.35 IU/ml, 0.4 IU/ml or more over a period of about 1-5 hours, 2-4 hours.

In a preferred embodiment, at least 5%, 10%, 15%, 20%, 30%, 40% or more of the M405 is delivered to the intestinal mucosa. In another embodiment, the M405 is delivered to the intestinal mucosa in an amount effective to produce a peak plasma concentration of the M405 within 1, 2, 3, 4, 5, 6, 7 hours after delivery. In yet another embodiment, at least 5%, 10%, 15%, 20%, 25%, 30% or more of the M405 is detectable in the blood within about 110, 2 to 7, 3 to 5 hours after delivery. In preferred embodiments, the bioavailability of the M405 is at least about 5%, 10%, 15%, 20%, 25%, 30%, 35%, 40%, 50%, 60%, 70%, 80% or more.

In one embodiment, the M405 is in the form of a solid. In other embodiments, the M405 is in aqueous form.

The M405 can also be included in a composition, e.g., a composition which includes a pharmaceutically acceptable carrier. The pharmaceutical composition can further include an active agent, e.g., an active agent distinct from the heparin (e.g., the heparin is a carrier), a delivery enhancer, etc.

In a preferred embodiment, the composition has a polydispersity of less than 1.3, 1.2, 1.1. or 1 (and integers therebetween).

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In another aspect, the invention features a method for delivering M108 to a subject, which includes orally administering to a subject a composition comprising M108 in an effective amount to deliver a therapeutic dose of M108 to the subject, thereby delivering M108 to the subject.

In one embodiment, the therapeutically effective amount of the M108 is about 1 mg/kg, 2 mg/kg, 3 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg.

In another embodiment, the M108 has an absorption rate in the gastrointestinal tract of about 0.2 IU/ml, 0.25 IU/ml, 0.3 IU/ml, 0.35 IU/ml, 0.4 IU/ml or more over a period of about 1-5 hours, 2-4 hours.

In a preferred embodiment, at least 5%, 10%, 15%, 20%, 30%, 40% or more of the M108 is delivered to the intestinal mucosa. In another embodiment, the M108 is delivered to the intestinal mucosa in an amount effective to produce a peak plasma concentration of the M108 within 2, 3, 4, 5, 6, 7 hours after delivery. In yet another embodiment, at least 5%, 10%, 15%, 20%, 25%, 30%, 40% or more of the M108 is detectable in the blood within about 1-10, 2 to 7, 3 to 5 hours after delivery. In preferred embodiments, the bioavailability of the M108 is at least about 5%, 10%, 15%, 20%, 25%, 30%, 35%, 40%, 50%, 60%, 70%, 80% or more.

In one embodiment, the M108 is in the form of a solid. In other embodiments, the M108 is in aqueous form.

The M108 can also be included in a composition, e.g., a composition which includes a pharmaceutically acceptable carrier. The pharmaceutical composition can further include an active agent, e.g., an active agent distinct from the heparin (e.g., the heparin is a carrier), a delivery enhancer, etc.

In a preferred embodiment, the composition has a polydispersity of less than 1.3, 1.2, 1.1. or 1 (and integers therebetween).

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In yet another aspect, the invention features a method for pulmonary delivery of heparin, e.g., LMWH, to a subject. The method includes administering a therapeutically effective amount of a heparin, e.g., LMWH, to pulmonary tissue of a subject, wherein the heparin, e.g., LMWH, has a net negative charge which is less than a reference net charge for the heparin, to thereby delivery the heparin to the subject.

In preferred embodiments, the heparin is a LMWH. Examples of such LMWH include those LMWH described herein, e.g., charge neutralized and/or mass reduced enoxaparin, dalteparin, reviparin, tinzaparin, nadroparin, certoparin, ardeparin, and parnaparin, as well as M312, M118, M405, M108, M115, and M411.

In a preferred embodiment, the LMWH has a net charge that is less than the net charge of enoxaparin, e.g., the net charge of the LMWH is at least 10%, 20%, 30%, 40%, 50%, 60%, 70%, 80%, 90%, 100% less than the net charge of enoxaparin. For example, the net charge of enoxaparin is about 19.23. Thus, in one embodiment, the LMWH has a net charge of less than about 19, 18, 17, 16, 15, 14, 13, 12, 11, 10, 9, 8, 7, 6, 5, 4, 3, 2, or 1. In another embodiment, the LMWH is a LMWH derived from nadroparin and the net charge of the LMWH is less than 27, 26, 25, 24, 23, 22, 21, 20, 19, 18, 17, 16, 15, 14, 13, 12, 11, 10, 9, 8, 7, 6, 5, 4, 3, 2, or 1. In another embodiment, the LMWH is derived from dalteparin and the net charge of the LMWH is less than about 22, 21, 20, 19, 18, 17, 16, 15, 14, 13, 12, 11, 10, 9, 8, 7, 6, 5, 4, 3, 2, or 1. In yet another embodiment, the LMWH is a LMWH derived from reviparin and the net charge of the LMWH is less than 25, 24, 23, 22, 21, 20, 19, 18, 17, 16, 15, 14, 13, 12, 11, 10, 9, 8, 7, 6, 5, 4, 3, 2, or 1. In another preferred embodiment, the heparin is a heparin derived from parnaparin and the net charge of the heparin is less than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1. In yet another preferred embodiment, the heparin is a heparin-derived from tinzaparin and the heparin has a net charge of less than 28, 27, 26, 25, 24, 23, 22, 21, 20, 19, 18, 17, 16, 15, 14, 13, 12, 11, 10, 9, 8, 7, 6, 5, 4, 3, 2, or 1.

In other embodiments, the heparin further has a mass which less than a reference mass for the heparin. For example, the heparin is a LMWH and the LMWH has a mass that is less than the mass of enoxaparin. Preferably, the mass of the LMWH is less than at least 5, 10, 15, 20, 25, 30, 35, 40, 50, 60, 70, 80, 90, 100, 150, 200, 250, 300, 350, 500, 1000, 1500, 2000, 2500 Da than the mass of enoxaparin. In other preferred embodiments, the mass of the LMWH is about 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700 Da or less.

In one embodiment, the heparin is a heparin derived from enoxaparin and the mass of enoxaparin is reduced such that the mass of the heparin is about 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000, 1900, 1800, 1700 Da or less. In another embodiment, the heparin is derived from nadroparin and the mass of nadroparin is reduced such that the mass of the heparin is about 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In other embodiments, the heparin is derived from dalteparin and the mass of dalteparin is reduced such that the mass of the heparin is about 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In yet another embodiment, the heparin is derived from reviparin and the mass of the reviparin is reduced such that the mass of the heparin is about 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In another embodiment, the heparin is derived from parnaparin and the mass of parnaparin is reduced such that the mass of the heparin is about 6,500, 6,400, 6,300, 6,200, 6,100, 6,000, 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less. In yet another embodiment, the heparin is derived from tinzaparin and the mass of tinzaparin is reduced such that the mass of the heparin is about 6,00, 5900, 5800, 5700, 5500, 5200, 5000, 4700, 4500, 4400, 4300, 4200, 4100, 4000, 3900, 3800, 3700, 3600, 3500, 3400, 3300, 3100, 3000, 2900, 2800, 2700, 2600, 2500, 2400, 2300, 2200, 2100, 2000 Da or less.

In one embodiment, the heparin is derived from enoxaparin and the enoxaparin is neutralized and the mass reduced such that the heparin has a charge to mass ratio that is less than about 19.32/4200. In another embodiment, the heparin is derived from nadroparin and the nadroparin is neutralized and the mass reduced such that the heparin has a charge to mass ratio that is less than about 27.6/6000. In yet other embodiments, the heparin can be, for example, a neutralized dalteparin having a charge to mass ratio of less than about 23/5000, or a neutralized reviparin having a charge to mass ratio of less than about 25.3/5500. In another embodiment, the heparin is derived from parnaparin and the parnaparin is neutralized and the mass reduced such that the heparin has a charge to mass ratio that is less than about 30.4/6610. In yet other embodiments, the heparin can be, for example, a neutralized tinzaparin having a charge to mass ratio of less than about 28.06/6100.

In one embodiment, the therapeutically effective amount of the LMWH is about 1 mg/kg, 2 mg/kg, 3 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg.

In one embodiment, the LMWH has an absorption rate in the pulmonary tissue of about 0.2 IU/ml, 0.25 IU/ml, 0.3 IU/ml, 0.35 IU/ml, 0.4 IU/ml or more over a period of about 10 minutes to 5 hours, 30 minutes to 3 hours, 1 to 2 hours. In another embodiment, at least 5%, 10%, 15%, 20%, 30%, 40%, 50%, 60% or more of the LMWH is delivered to the pulmonary tissue, e.g., the upper and/or lower respiratory tract (e.g., deep lung). In yet other embodiments, the LMWH is delivered to the pulmonary tissue in an amount effective to produce a peak plasma concentration of the LWMH within 10 minutes to 3 hours, 30 minutes to 2 hours, after delivery. In another embodiment, at least 5%, 10%, 15%, 20%, 25%, 30% or more of the LMWH is detectable in the blood within about 5 minutes to 5 hours, 10 minutes to 4 hours, 30 minutes to 2 hours after delivery.

In one embodiment, the LMWH is in the form of a solid, e.g., a dry particle. When the LMWH is in the form of a dry particle, the particle can have a mean geometric diameter of 1 to 500 microns (and integers therebetween). In another embodiment, the LMWH is in aqueous form.

The LMWH can also be included in a composition, e.g., a composition which includes a pharmaceutically acceptable carrier. The pharmaceutical composition can further include an active agent, e.g., an active agent distinct from the LMWH (e.g., the LMWH is a carrier), a delivery enhancer, etc.

In a preferred embodiment, the composition has a polydispersity of less than 1.3, 1.2, 1.1. or 1 (and integers therebetween).

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In yet another aspect, the invention features a method for delivery of an HLGAG, e.g., a synthetic HLGAG, e.g., a synthetic pentasaccharide, e.g., Arixtra, to the pulmonary system of a subject. The method includes administering a therapeutically effective amount of an HLGAG, to pulmonary tissue of a subject to provide a preselected effect, e.g., anti-Xa and/or anti-IIa activity, in the subject. In a preferred embodiment, the method includes administering a dose of the HLGAG to the pulmonary system where the dose is at least 2, preferably, 3, 4 or 5 times greater than a subcutaneous or intravenous dose of the HLGAG which is effective to give the preselected therapeutic effect, e.g., anti-Xa and/or anti-IIa activity.

In one embodiment, the therapeutically effective unit dose of the HLGAG, e.g., the synthetic HLGAG, is about 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 3 mg/kg or doses therebetween. In another embodiment, the therapeutically effective unit dose of the HLGAG, e.g., the synthetic HLGAG is about 3 mg, 4 mg, 5 mg, 6 mg, 7 mg, 8 mg, 16 mg, 48 mg, 80 mg, 120 mg or doses therebetween. In a preferred embodiment, the synthetic HLGAG is Arixtra. In other embodiments, the synthetic HLGAG is one or more of the compounds provided in FIG. 9 and derivatives thereof. In one embodiment, the synthetic HLGAG is one or more of the compounds provided in FIG. 9 and derivatives thereof, and the therapeutically effective dose is about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 3 mg/kg or doses therebetween

In another embodiment, the therapeutically effective unit doses of the HLGAG, e.g., the synthetic HLGAG, is amount effective to produce a peak plasma concentration of the HLGAG, e.g., the synthetic HLGAG, within about 5 minutes to about 5 hours, 10 minutes to about 3 hours, 30 minutes to about 2 hours, after delivery.

In one embodiment, the HLGAG, e.g., the synthetic HLGAG is in the form of a solid, e.g., a dry particle. In other embodiments, the HLGAG, e.g., the synthetic HLGAG, is in the form of a liquid.

In one embodiment, the HLGAG is Arixtra and the therapeutically effective unit dose is about 0.01 mg/kg, 0.03 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg or doses therebetween. In another embodiment, the therapeutically effective unit dose of the Arixtra is about 8 mg, 16 mg, 48 mg, 80 mg, 120 mg or doses therebetween.

In another embodiment, the HLGAG is Arixtra and the therapeutically effective unit doses is amount effective to produce a peak plasma concentration of the Arixtra within about 5 minutes to about 5 hours, 10 minutes to about 3 hours, 30 minutes to about 2 hours, after delivery.

In one embodiment, the HLGAG, e.g., the synthetic HLGAG, e.g., the synthetic pentasaccharide, has an absorption rate in the pulmonary tissue of about 0.2 IU/ml, 0.25 IU/ml, 0.3 IU/ml, 0.35 IU/ml, 0.4 IU/ml, 0.5 IU/ml, 0.7 IU/ml, 0.9 IU/ml, 1 IU/ml, 1.5 IU/ml, 2 IU/ml or more over a period of about 10 minutes to 5 hours, 30 minutes to 3 hours, 1 to 2 hours. In another embodiment, at least 5%, 10%, 15%, 20%, 30%, 40%, 50%, 60% or more of the HLGAG, e.g., the synthetic HLGAG, e.g., the synthetic pentasaccharide, is delivered to the pulmonary tissue, e.g., the upper and/or lower respiratory tract (e.g., deep lung). In yet other embodiments, the HLGAG, e.g., the synthetic HLGAG, e.g., the synthetic pentasaccharide, is delivered to the pulmonary tissue in an amount effective to produce a peak plasma concentration of the HLGAG within 5 minutes to 5 hours, 10 minutes to 3 hours, 30 minutes to 2 hours, after delivery. In another embodiment, at least 5%, 10%, 15%, 20%, 25%, 30% or more of the HLGAG, e.g., the syntetic HLGAG, e.g., the synthetic pentasaccharide is detectable in the blood within about 5 minutes to 5 hours, 10 minutes to 4 hours, 30 minutes to 2 hours after delivery.

In one embodiment, the HLGAG is in the form of a solid, e.g., a dry particle. In another embodiment, the HLGAG is in the form of a liquid.

The HLGAG, e.g., the synthetic HLGAG, e.g., the synthetic pentasaccharide, can also be included in a composition, e.g., a composition which includes a pharmaceutically acceptable carrier. The pharmaceutical composition can further include an active agent, e.g., an active agent distinct from the HLGAG, e.g., a delivery enhancer (e.g., a surfactant), etc.

In another embodiment, the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; obesity or excess adipose, an allergy; a respiratory disorder, such as asthma, emphysema, adult respiratory distress syndrome (ARDS), cystic fibrosis, or lung reperfusion injury; a cancer or metastatic disorder, e.g., lipomas; diabetes; an angiogenic disorder, such as neovascular disorders of the eye, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, coronary artery bypass graft surgery (CABG).

In yet another aspect, the invention features a method for delivery of an HLGAG to provide a therapeutic effect, e.g., an anti-Xa and/or anti-IIa activity, which includes administering a dose of the HLGAG to the pulmonary system wherein the HLGAG is

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph depicting anti-Xa activity assay of two enoxaparin-derived particle preparations in plasma samples.

FIG. 2 is a graph depicting absorption rates using an anti-Xa activity assay of enoxaparin-derived particles administered by inhalation to rabbits with an insufflator at 3 and 6 mg/kg via an intubated trachea tube.

FIG. 3 is a graph depicting anti-Xa activity assay of two enoxaparin-derived particle preparations having distinct formulations (described in Examples) in plasma samples.

FIG. 4 is a graph depicting absorption rates using an anti-Xa activity assay of daltaparin-derived particles administered by inhalation to rabbits with an insufflator at 3 and 6 mg/kg via an intubated trachea tube.

FIG. 5 is a graph depicting anti-Xa activity assay of two dalteparin-derived particle preparations having distinct formulations (described in Examples) in plasma samples.

FIG. 6 is a graph depicting the absorption rates of various LMWH having differing charge to mass ratios. Absorption rates were determined using anti-Xa activity of three the LMWH, namely enoxaparin, M108 and M405, administered by intraduodeni delivery.

FIG. 7 is a graph depicting the absorption rates of two LMWH having different polydispersities. Absorption rates were determined using anti-Xa activity of two LMWH, enoxaparin (having a polydispersity of 1.35) and M405 (having a polydispersity of 1) administered by intraduodenl delivery.

FIG. 8 is a graph depicting anti-Xa activity of Arixtra at doses of 0.23 mg/kg, 0.45 mg/kg and 0.6 mg/kg administered by inhalation to rabbits with an insufflator via an intubated trachea tube.

FIG. 9 depicts the structure of several synthetic HLGAGs.

FIG. 10 is a graph depicting venous thrombosis inhibition by a LMWH delivered via inhalation or subcutaneous delivery. Sprague-Dawley rats (n=3) were treated with ardeparin (3 mg/kg), either via subcutaneous injection or via inhalation. Animals were monitored for 2 hours (subcutaneous injection) or for 15 min (inhalation), upon which Russell's Viper Venom was injected. After 2 minutes, the inferior vena cava was ligated and thrombus formation was monitored.

FIG. 11 is a graph depicting the effect of different formulations on administration of a LMWH by inhalation. Different particles of ardeparin: Rabbits (n=3) were intubated with a tracheal tube and heparin, formulated with different percentages of lactose, was delivered into the lung using an insufflator. The concentration of heparin in auricular blood was measured using an automated chromogenic method.

FIG. 12 is a graph depicting the effect of different formulations on administration of a LMWH by inhalation: Rabbits (n=3) were intubated with a tracheal tube and heparin, formulated with lactose or DPPC, was delivered into the lung using an insufflator. The concentration of heparin in auricular blood (0.3 ml) was measured using an automated chromogenic method.

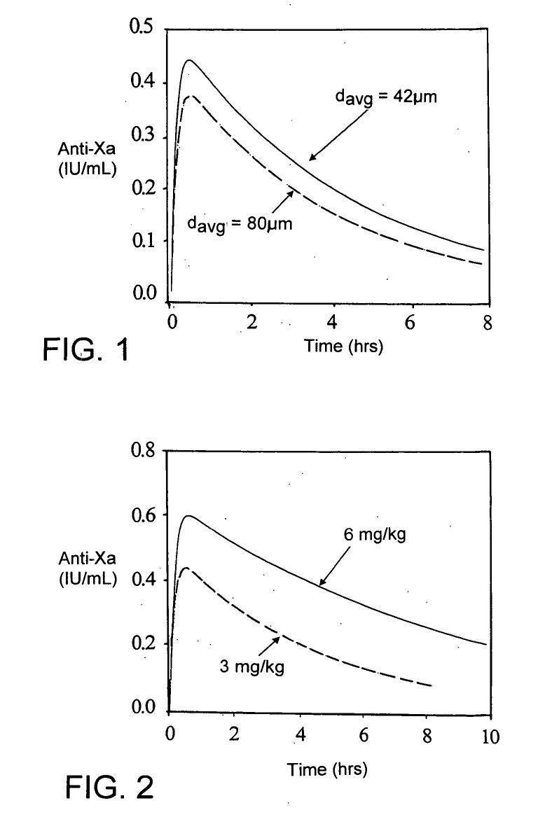

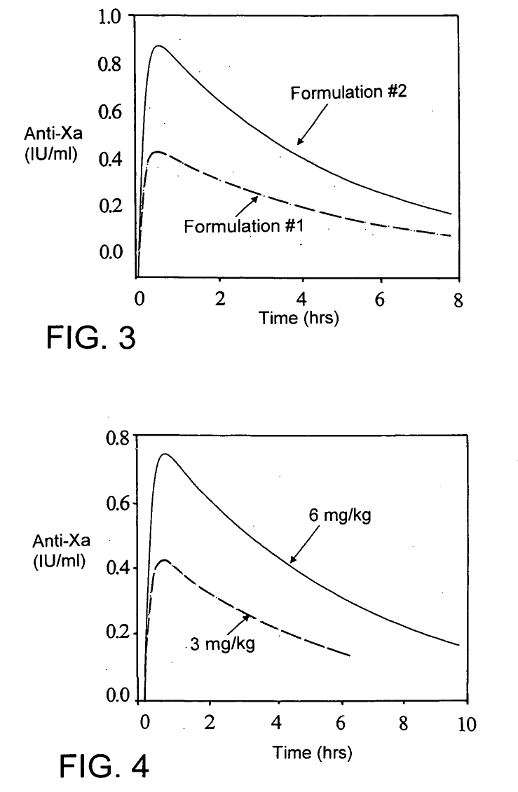

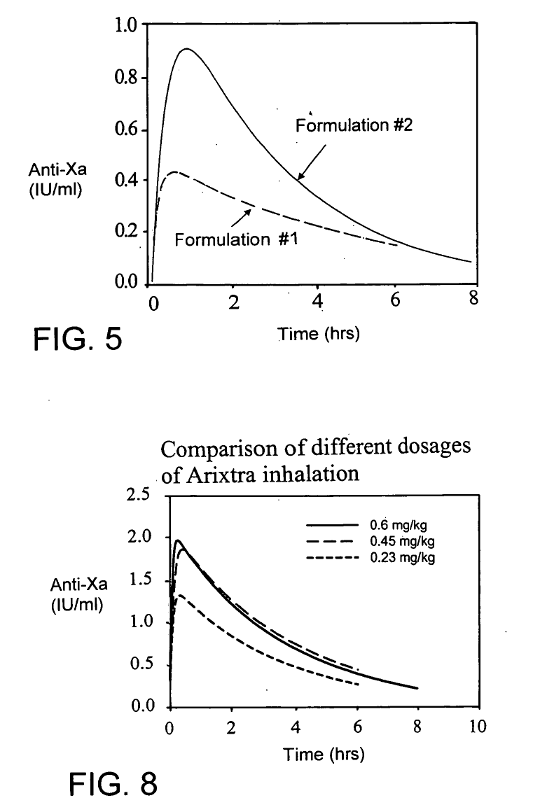

### DETAILED DESCRIPTION OF THE INVENTION

It was discovered that polysaccharides such as heparin and low molecular weight heparin (LMWH) are amenable to non-invasive delivery at therapeutically effective levels. In addition, it was found that by identifying chemical properties of polysaccharides, enhanced formulations for non-invasive in vivo delivery can be generated.

The methods described herein can be used to generate polysaccharide compositions which have high levels of bioavailability by administrative routes which in the past had met with limited success, if any. As shown in the Examples, HLGAGs have been generated which have enhanced oral and pulmonary delivery profiles.

A “polysaccharide” as used herein is a polymer composed of monosaccharides linked to one another. In many polysaccharides, the basic building block of the polysaccharide is actually a disaccharide unit, which can be repeating or non-repeating. Thus, a unit when used with respect to a polysaccharide refers to a basic building block of a polysaccharide and can include a monomeric building block (monosaccharide) or a dimeric building block (disaccharide). Polysaccharides include but are not limited to heparin-like glycosaminoglycans, chondroitin sulfate, hyaluronic acid and derivatives or analogs thereof, chitin derivatives and analogs thereof, e.g., 6-0-sulfated carboxymethyl chitin, immunogenic polysaccharides isolated from phellinus linteus, PI-88 (a mixture of highly sulfated oligosaccharide derived from the sulfation of phosphomannum which is purified from the high molecular weight core produced by fermentation of the yeast pichia holstii) and its derivatives and analogs, polysaccharide antigens for vaccines, and calcium spirulan (Ca-SP, isolated from blue-green algae, spirulina platensis) and derivatives and analogs thereof.

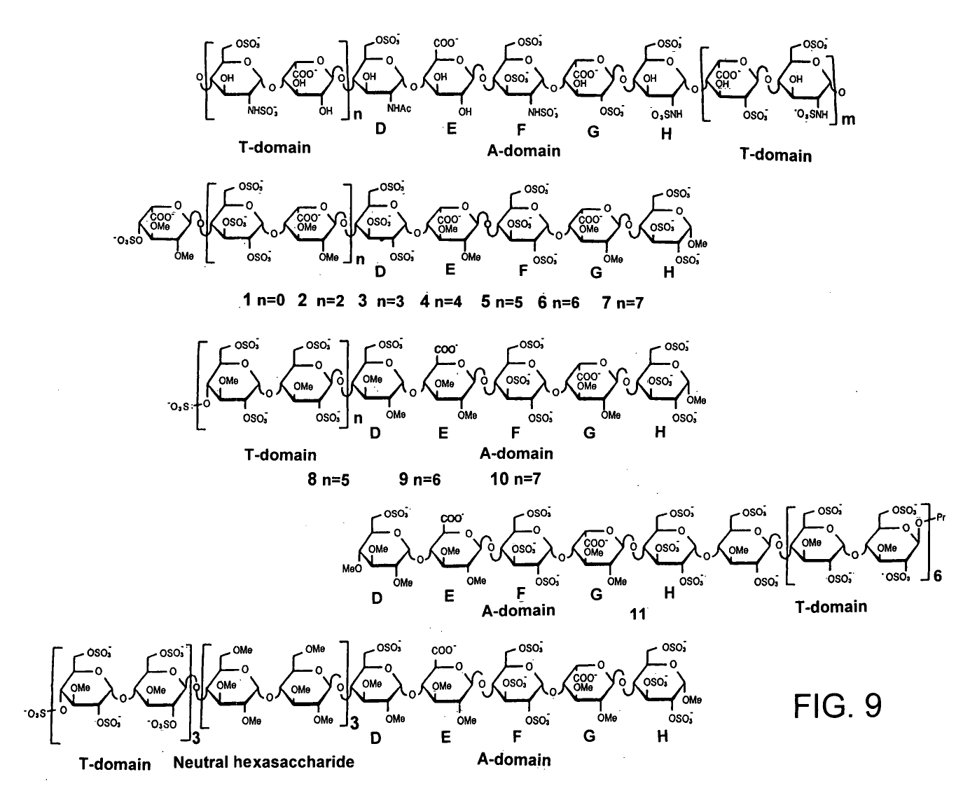

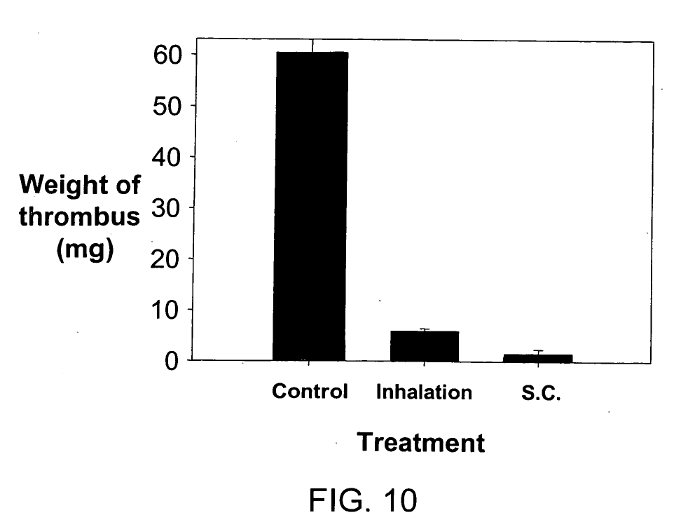

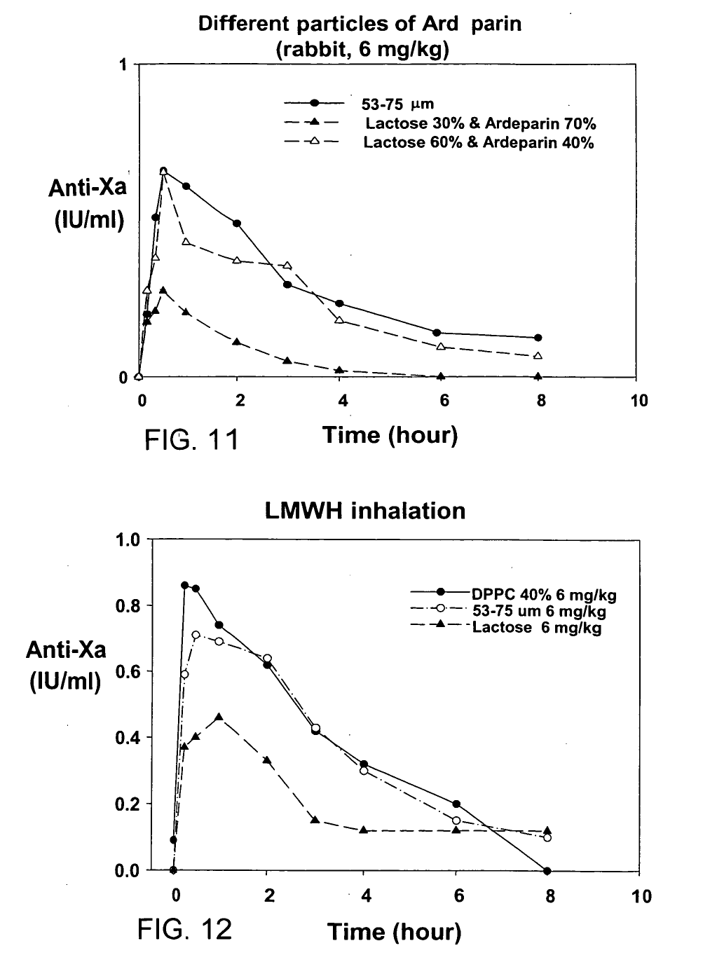

One preferred type of polysaccharide is an HLGAG. Thus, in some embodiments, the active agent being delivered is a polysaccharide such as heparin-like glycosaminoglycans (HLGAGs). The methods taught herein are sometimes described with reference to HLGAGs but the properties taught herein can be extended to other polysaccharides, and unless a claim specifies otherwise the claims encompass any polysaccharide and optionally, a polysaccharide having a diagnostic, prophylactic, or therapeutic utility. As used herein the terms “HLGAG” and “glycosaminoglycans” are used interchangeably to refer to a family of molecules having heparin like structures and properties. These molecules include but are not limited to low molecular weight heparin (LMWH), heparin, biotechnologically prepared heparin, chemically modified heparin, synthetic heparin such as pentasaccharides (e.g., Arixtra) and the structures depicted in FIG. 9, heparin mimetics and heparan sulfate. The term “biotechnological heparin” encompasses heparin that is prepared from natural sources of polysaccharides which have been chemically modified and is described in Razi et al., Bioche. J. 1995 Jul. 15;309 (Pt 2): 465-72. Chemically modified heparin is described in Yates et al., Carbohydrate Res (1996) November 20;294:15-27, and is known to those of skill in the art. Synthetic heparin is well known to those of skill in the art and is described in Petitou, M. et al., Bioorg Med Chem Lett. (1999) April 19;9(8):1161-6 and Vlodavsky et al., Int. J. Cancer, 1999, 83:424-431. An example of a synthetic heparin is fondaparinux. Fondaparinux (Arixtra) is a 5 unit synthetic glycosaminoglycan corresponding to the AT-III binding site. Heparan Sulfate refers to a glycosaminoglycan containing a disaccharide repeat unit similar to heparin, but which has more N-acetyl groups and fewer N- and O-sulfate groups. Heparin mimetics are monosaccharides (e.g., sucralfate), oligosaccharides, or polysaccharides having at least one biological activity of heparin (i.e., anticoagulation, inhibition of cancer, treatment of lung disorders, etc.). Preferably these molecules are highly sulfated. Heparin mimetics may be naturally occurring, synthetic or chemically modified. (Barchi, J. J., Curr. Pharm. Des., 2000, March, 6(4):485-501). The term “HLGAG” also encompasses functional variants of the above-described HLGAG molecules. These functional variants have a similar structure but include slight modifications to the structure which allow the molecule to retain most of its biological activity or have increased biological activity.

“LMWH” as used herein refers to a preparation of sulfated glycosaminoglycans (GAGs) having an average molecular weight of less than 8000 Da, with about at least 60% of the oligosaccharide chains of a LMWH preparation having a molecular weight of less than 8000 Da. Several LMWH preparations are commercially available, but, LMWHs can also be prepared from heparin, using e.g., HLGAG degrading enzymes. HLGAG degrading enzymes include but are not limited to heparinase-I, heparinase-II, heparinase-III, heparinase IV, heparanase, D-glucuronidase and L-iduronidase. The three heparinases from Flavobacterium heparinum are enzymatic tools that have been used for the generation of LMWH (5,000-8,000 Da) and ultra-low molecular weight heparin (˜3,000 Da). Commercially available LMWH include, but are not limited to, enoxaparin (brand name Lovenox; Aventis Pharmaceuticals), dalteparin (Fragmin, Pharmacia and Upjohn), certoparin (Sandobarin, Novartis), ardeparin (Normiflo, Wyeth Lederle), nadroparin (Fraxiparine, Sanofi-Winthrop), parnaparin (Fluxum, Wassermann), reviparin (Clivarin, Knoll A G), and tinzaparin (Inohep, Leo Laboratories, Logiparin, Novo Nordisk). Some preferred forms of LMWH include enoxaparin (Lovenox) and dalteparin (Fragmin). The term “Arixtra” as used herein refers to a composition which includes a synthetic pentasaccharide of methyl O-2-deoxy-6-O-sulfo-2-(sulfoamino)-α-D-glucopyranosyl-(1→4)-O-β-D-glucopyranosyl-(1→4)-O-2-deoxy-3,6-di-O-sulfo-2-(sulfoamino)-α-D-glucopyranosyl-(1→4)-O-2-O-sulfo-α-L-idopyranuronosyl-(1→4)-2-deoxy-6-O-sulfo-2-(sulfoamino)-α-D-glucopyranoside, decasodium salt and derivatives thereof. A “synthetic heparin” or “synthetic HLGAG” as used herein refers to HLGAGs are synthesized compounds and are not derived by fragmentation of heparin. Examples of synthetic heparins are provided in FIG. 9. Methods of preparing synthetic heparins are provided, for example, in Petitou et al. (1999) Nature 398:417, the contents of which is incorporated herein by reference. The term synthetic heparins also includes derivatives of the heparins provided in FIG. 9.

Many currently available polysaccharides such as heparins are polydisperse mixtures which contain a large number of chains having a range of different molecular weights. The term “polydisperse” or “polydispersity” refers to the weight average molecular weight of a composition (M_{w}) divided by the number average molecular weight (Mₙ). The number average molecular weight (Mₙ) is calculated from the following equation: Mₙ=Σcᵢ/(Σcᵢ/mᵢ). The variable cᵢ is the concentration of the polymer in slice i and Mᵢ is the molecular weight of the polymer in slice i. The summations are taken over a chromatographic peak, which contains many slices of data. A slice of data can be pictured as a vertical line on a plot of chromatographic peak versus time. The elution peak can therefore be divided into many slices. The number average molecular weight is a calculation dependent on the molecular weight and concentration at each slice of data. The weight average molecular weight (M_{w}) is calculated from the following equation: M_{w}=Σ(cᵢ/mᵢ)/Σcᵢ. The weight average molecular weight calculation is average dependant on the summation of all slices of the concentration and molecular weight. The polydispersity of unfractionated heparin and various LMWHs are known, as are methods for determining polydispersity. As an example, a preparation of unfractionated heparin has a polydispersity of about 1.5 to 2.0. Many commercially available LMWH such as enoxaparin, dalteparin and reviparin are also available as polydisperse mixtures of LMWH chains having a wide range of molecular weight chains present. Enoxaparin, for example, has an average molecular weight of 4,200 daltons. However, a preparation of enoxaparin has a molecular weight distribution where less than or equal to 20% of the preparation has a molecular weight of less than 2000 daltons, greater than or equal to 68% of the preparation has a molecular weight between 2000 and 8000 daltons, and less than or equal to 18% of the preparation has a molecular weight of greater than 8000 daltons. Similar molecular weight distributions exist for other commercially available LMWH such as dalteparin, which has an average molecular weight of about 5000 daltons and has the following molecular weight distribution: about 3-15% of the preparation has a molecular weight of less than 3000 daltons, about 65 to 78% of the preparation has a molecular weight of between 3000 and 8000 daltons, and about 14 to 26% of the preparation has a molecular weight of greater than 8000 daltons, and reviparin, which has an average molecular weight of about 5500 to 7500 daltons and has the following molecular weight distribution: about <10% of the preparation has a molecular weight of less than 2000 daltons, about 60 to 72% of the preparation has a molecular weight of between 2000 and 8000 daltons, and about 22 to 36%% of the preparation has a molecular weight of greater than 8000 daltons. High levels of polydispersity can make the pharmacokinetics of such polysaccharide compositions complicated. Thus, compositions with a lower polydispersity are desirable for therapeutics so that the composition results in a more consistent end product with a more consistent therapeutic effect.

Accordingly, in one aspect, the invention includes polysaccharide compositions for in vivo delivery which have a polydispersity less than that of a reference value, e.g., a reference value for unfractioned heparin or a LMWH such as enoxaparin. Preferably, the polydispersity of the polysaccharide composition is less than 1.0, 0.9, 0.8, 0.7, 0.6, 0.5, 0.4, or 0.3 of the reference value. For example, the polydispersity of a LMWH composition is preferably at least 0.01, 0.1, 0.2, 0.3 (and integers there between) lower than the referenced polydispersity value for enoxaparin (i.e., 1.35). In other aspects, the polydispersity of the finished polysaccharide composition is less than the starting material. For example, the polydispersity of a digested, e.g., enzymatically or chemically digested, heparin is preferably less than the unfractionated heparin from which it was derived, e.g., the polydispersity is 0.01, 0.1, 0.2, 0.3, 0.4, 0.5, 0.6, 0.7, 0.8, 0.9 (and integers there between) or lower than the referenced polydispersity value for unfractionated heparin (i.e., 1.5 to 2.0). As shown herein, lower polydispersity in a polysaccharide composition, e.g., a heparin or LMWH composition can enhance the delivery of the polysaccharide by non-invasive routes. Thus, enhanced delivery profiles, e.g., non-invasive delivery profiles, can be obtained by providing a polysaccharide composition with low polydispersity.

Methods of Neutralizing the Net Charge and/or Mass of a Polysaccharide

The enhanced delivery profiles, e.g., for transdermal, pulmonary and mucosal delivery, described herein, can be generated by neutralizing a polysaccharide and/or by reducing the mass of the polysaccharide based, e.g., upon its chemical signature. Using the chemical signature of the polysaccharide, charges can be neutralized and/or the mass of the polysaccharide reduced.

A “neutralized formulation” as used herein is a formulation in which the net negative or positive charge has been reduced or masked by at least 10%. In other embodiments, the neutralized formulation is a formulation in which the net negative or positive charge has been reduced by at least 20%, 30%, 40%, 50%, 60%, 70%, 80, 90% or 100% or any integer there between. A “completely neutral” formulation is one in which there is a net negative and positive charge of zero.

In addition to the specific HLGAG compositions, it was discovered that specific chemical properties of a polysaccharide may be identified and manipulated in order to enhance delivery of active agents by non-invasive routes, such as transdermal, pulmonary and mucosal routes. The chemical properties of the polysaccharide may be altered by various techniques in order to enhance delivery of diagnostic or therapeutic polysaccharides or of active agents (e.g., therapeutic polypeptides) associated with carrier polysaccharides. Methodologies have been developed to determine chemical signatures of polysaccharides. A chemical signature, as used herein, refers to information regarding, e.g., the identity and number the mono- and di-saccharide building blocks of a polysaccharide, information regarding the physiochemical properties such as the overall (also referred to as the “net charge”), charge density, molecular size, charge to mass ratio and the presence of iduronic and/or glucuronic acid content as well as the relationships between the mono- and disaccharide building blocks, and active sites associated with these building blocks. As described herein, it is possible to use specific chemical signatures to formulate polysaccharides with enhanced, e.g., transdermal, pulmonary and/or mucosal, delivery properties. The chemical signature can be provided by determining one or more primary outputs chosen from the following: the presence or the amount of one or more component saccharides or disaccharides; the presence or the amount of one or more block components, wherein a block component is one made up of more than one saccharides or polysaccharide;

the presence or amount of one or more saccharride-representative, wherein a saccharride-representative is a saccharride modified to enhance detectability; the presence or amount of an indicator of three dimensional structure or a parameter related to three dimensional structure, e.g., activity, e.g., the presence or amount of a structure produced by cross-linking a polysaccharide, e.g., the cross-linking of specific saccharrides which are not adjacent in the linear sequence; or the presence or amount of one or more modified saccharides, wherein a modified saccharide is one present in a starting material used to make a preparation but which is altered in the production of the preparation, e.g., a saccharide modified by cleavage. The chemical signature can also be provided by determining a secondary output, which include one or more of: total charge; density of charge.

As used herein, “1” or “peak 1” refers to ΔU_{2S}H_{NS,6S}; “2” or “peak 2” refers to ΔU_{2S}H_{NS}; “3” or “peak 3” refers to ΔUH_{NS,6S}; “4” or “peak 4” refers to ΔU_{2S}H_{NAC,6S}; “5” or “peak 5” refers to ΔUH_{NS}; “6” or “peak 6” refers to ΔU_{2S}H_{NAC}; “7” or “peak 7” refers to ΔUH_{NAC,6S}; “8” or “peak 8” refers to ΔUH_{NAC,6S}GH_{NS,3S,6S}; ΔUH_{NS,6S}GH_{NS,3S,6S}; ΔU H_{NAC,6S}GH_{NS,3S}; or ΔU H_{NS,6S}GH_{NS,3S}, collectively. The nomenclature “ΔU” refers to an unsaturated Uronic acid (Iduronic acid (I) or Glucuronic acid (G) that has a double bond introduced at the 4-5 position as a result of the lyase action of heparinases. Upon the introduction of the double bond the distinction between the stereo isomers I and U disappears, and hence the notation ΔU: Δ to denote double bond, and U to denote that they can be derived from either I or U. Thus, as used herein, “ΔU” represents both I and G, such that ΔU_{2S}H_{NS,6S} encompasses both I_{2S}H_{NS,6S} and G_{2S}H_{NS,6S}; ΔU_{2S}H_{NS} encompasses both I_{2S}H_{NS} and G_{2S}H_{NS}, and so forth.

The process of identifying chemical properties or signatures of a polysaccharide and using this information to generate polysaccharides for enhanced in vivo delivery is referred to herein as the process of chemical formulation of a polysaccharide. Chemical formulation involves the preparation of a composition using chemical entities to achieve an appropriate balance for delivery. The chemical formulation is accomplished using techniques to structurally characterize or sequence polysaccharides and then formulating, e.g., effectively masking charge based on the structure. This is distinct from physical formulation of a polysaccharide, which refers to the processing of a particle by methods known in the art based on the physical attributes of the particle such as particle size, tap density, etc. that are all physical descriptions of particles. The compositions and methods of the invention involve at a minimum chemical formulation of polysaccharides for efficient transdermal, pulmonary and mucosal delivery. In addition to the chemical formulation, the polysaccharides may be physically formulated to achieve, e.g., a particular particle size, tap density etc. It has been found that such chemical formulations can enhance non-invasive delivery without being physically formulated. One specific chemical property that may be analyzed is charge. Neutralization of the charge of a polysaccharide enhances the ability the polysaccharide to permeate lipid membranes, e.g., intestinal and alveolar membranes. As used herein the terms “neutralization”, “neutralize” and “neutralizing” refer a process for generating a polysaccharide in which the net negative or positive charge of the material has been reduced or masked by at least 10% and in some embodiments by at least 20%, 30%, 40%, 50%, 60%, 70%, 80, 90% or 100 or any integer inbetween. The net or overall charge of a polysaccharide such as heparin can be calculated by dividing the mass of the heparin by the average molecular weight of a disaccharide (500) and multiplying that number by the average charge per disaccharide (e.g., 2.3). The average charge per disaccharide can vary from polysaccharide to polysaccharide. The average charge is the mean charge for the polysaccharides present in a polydisperse composition. The net charge of each polysaccharide in a composition can vary. Methods of determining the charge of polysaccharides including the charge per disaccharide are described, for example, in Venkataraman, G. et al. Science, 286, 537-542 (1999). Charge neutralization may be accomplished in a variety of ways. Preferably, the charge of the polysaccharide is determined. Based on that determination, an appropriate strategy for charge neutralization may be selected, e.g., a strategy which maintains one or more of the activities of the polysaccharide. In general, a more highly charged polysaccharide will be more effectively neutralized with the use of a higher concentration of neutralizing agent to mask the charge. For instance, chemical analysis of a heparin oligosaccharide revealed that the molecule contained a total of 17 negative charges, primarily O-sulfates. Charge neutralization and powder formation of the heparin molecule was accomplished by precipitating the polysaccharide using a 200 mM sodium chloride pH 4.5 solution. Similarly, a heterogeneous population of heparin, such as a low molecular weight heparin was chemically analyzed and found to have an average charge distribution of 24-32 negative charges. Charge neutralization and optimal powder formation of this material was accomplished by using a higher concentration of salt, counterions, and/or a different pH to effectively mask charge.

The neutralization may be accomplished using a charge neutralization agent. A “charge neutralization agent” as used herein is a positively or negatively charged compound that is capable of interacting with an oppositely charged molecule and thereby neutralizing the charge. Charge neutralization agents include but are not limited to counter ions such as mono- and divalent ions including, but not limited to, barium, calcium, sodium, potassium, lithium, ammonium, magnesium and zinc as well as transition metals such as iron, nickel, and copper; and other neutralizing compounds such as small organic compounds, spermine, spermidine, low molecular weight protamine, or basic peptides.

If a polysaccharide is negatively charged, a positively charged compound may be used to neutralize the polysaccharide. Likewise, if the polysaccharide is positively charged, then a negatively charged compound may be used. Once the type and quantity of charge in the polysaccharide is determined by chemical analysis then the appropriate amount of neutralizing compound may be selected. The exact amount neutralizing compound will depend on the particular sample, since the type and amount of charge may vary from sample to sample. In general, a low concentration of neutralizing agent will be sufficient to reduce the charge of a polysaccharide having only a few charged moieties and it is desirable to increase the concentration of the neutralizing agent for more highly charged molecules.

Another chemical property of the polysaccharides that may be considered is the quantity of 2-O sulfated iduronic acid moieties present in the polysaccharide. 2-O sulfated iduronic acid moieties chelate metals in a distinctly different matter than other components of a polysaccharide. As such the nature and amount of counter ions useful for neutralization is somewhat determined by the number and localization of 2-O sulfated iduronic acids in the polysaccharide. For instance, a heparin with a high degree of 2-O sulfated iduronic acid (˜80%) was efficiently precipitated using calcium or barium salts instead of sodium salts whereas a heparan sulfate with a low degree of 2-O sulfated iduronic acid was not precipitated in an appropriate manner using these same conditions. In general, a higher degree of 2-O sulfated iduronic acids in a polysaccharide is more effectively formulated with a higher concentration of neutralizing agents.

Additionally, the length of the polysaccharide has an impact on its formulation. Based on the length of the polysaccharide, different types and concentrations of organic modifiers such as organic solvents will have different effects on the formulation properties of the polysaccharide. For instance, different sized heparin oligosaccharides were demonstrated to form optimal powders at various concentrations of organic solvent. In general, the longer an oligosaccharide chain, and the higher its number of charges, the less soluble a polysaccharide is in non-aqueous solutions. As such, based on size and charge density as chemical signatures, powders can be formed via the addition of various volume equivalents of organic modifiers. In general, the longer an oligonucleotide within a particular class of polysaccharides (i.e., HLGAGs) a lower concentration of organic modifier will produce enhanced results.

An organic modifier as used herein is an organic solution such as, for instance, an alcohol and a polar organic solvent, such as acetonitrile, acetone, or dimethylsulfoxide and aqueous mixtures thereof.

The mass and/or charge of a polysaccharide can be reduced by digesting the polysaccharide with at least one agent. The agent can be selected, e.g., based upon the information obtained regarding the chemical signature of the polysaccharide. For example, enzymes and/or chemicals can be used which selectively cleave the polysaccharide. Thus, polysaccharides can be generated such that, e.g., regions of the polysaccharide which are not involved and/or do not influence a desired biological activity can be cleaved, and regions of the polysaccharide which are involved and/or influence a biological activity remain intact. As used herein, the term “intact” means uncleaved and complete.

For example, a LMWH can be generated which maintains at least one activity of heparin, e.g., anti-Xa and/or anti-IIa activity, but has a reduction in another activity, e.g., PF4 binding. The term “maintained” as used herein refers to the ability of the polysaccharide to be able to still perform the desired activity, even if the ability to perform that activity is reduced as compared to the activity of the polysaccharide prior to charge neutralization and/or mass reduction. Examples of activities mediated by heparin include: anti-Xa activity, anti-IIa activity, PF4 binding (or other measure of HIT propensity), FGF-binding, protamine neutralization, anticoagulation/antithrombosis, cell proliferation, e.g., unwanted cell proliferation, e.g., unwanted malignant or non-malignant cell proliferation; angiogenesis; inflammatory processes; cell migration; cell activation; cell adhesion. Standard methods of measuring such activities are known. For example, anti-Xa activity can be measured by the amidolytic method on a chromogenic substrate described by Teien et al., Thrombo. Res. 10:399-410 (1977), with a standard being the first international standard for LMWH. Known methods for measuring anti-IIa activity are described, for example, by Anderson et al., Thrombo. Res. 15:531-541 (1979), with a standard being the first international standard for LMWH.

HLGAG fragments may be degraded using for example, enzymes such as heparin lyase enzymes (heparinases) or nitrous acid. They may also be modified using different enzymes that transfer sulfate groups to the specific positions or remove the sulfate groups from those positions. The modifying enzymes are exolytic and nonprocessive which means that they just act once on the non-reducing end and will let go of the heparin chain without sequentially modifying the rest of the chain. For each of the modifiable positions in the disaccharide unit there exits a modifying enzyme. An enzyme that adds a sulfate group is called a sulfotransferase and an enzyme that removes a sulfate group is called a sulfatase. The modifying enzymes include 2-O sulfatase/sulfotransferase, 3-O sulfatase/sulfotransferase, 6-O sulfatase/sulfotransferase and N-deacetylase-Nsulfotransferase. The function of these enzymes is evident from their names, for example a 2-O sulfotransferase transfers a sulfate group to the 2-O position of an iduronic acid (2-O sulfated glucuronic acid is a rare occurrence in the HLGAG chains) and a 2-O sulfatase removes the sulfate group from the 2-O position of an iduronic acid.

HLGAG degrading enzymes include but are not limited to heparinase-I, heparinase-II, heparinase-III, heparinase-IV, heparanase, D-glucuronidase and L-iduronidase, modified versions of heparinases, variants and functionally active fragments thereof. The three heparinases from Flavobacterium heparinum are enzymatic tools that have been used for the generation of LMWH (5,000-8,000 Da) and ultra-low molecular weight heparin (≦3,000 Da). Heparinase I cleaves highly sulfated regions of HLGAGs at 2-O sulfated uronic acids, whereas heparinase II has a broader substrate specificity and cleaves glycosidic linkages containing both 2-O sulfated and nonsulfated uronic acids (Ernst, S., Langer, R., Cooney, C. L. & Sasisekliaran, R. (1995) Crit Rev Biochem Mol Biol 30, 387-444). Heparinase III, as opposed to heparinase I, cleaves primarily undersulfated regions of HLGAGs, viz., glycosidic linkages containing a nonsulfated uronic acid (Ernst, S., Langer, R., Cooney, C. L. & Sasiseldiaran, R. (1995) Crit Rev Biochem Mol Biol 30, 387-444). Several patents and patent applications describe useful modifications and variants and fragments of heparinase, including U.S. Pat. No. 6,217,863 and pending application Ser. Nos. 09/384,959 and 09/802,285. Other modifications and variants are also useful.

Glucuronidase and iduronidase, as their name suggests, cleave at the glycosidic linkage after a glucuronic acid and iduronic acid respectively. Nitrous acid clips randomly at glycosidic linkages after a N-sulfated hexosamine and converts the six membered hexosamine ring to a 5-membered anhydromannitol ring.

Chemicals useful for digesting polysaccharides such as HLGAGS include chemicals chosen from group consisting of oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

Methods for identifying the charge and other properties of polysaccharides have been described in Venkataraman, G., et al., Science, 286, 537-542 (1999), and U.S. patent application Ser. Nos. 09/557,997 and 09/558,137, both filed on Apr. 24, 2000, which are hereby incorporated by reference.

Formulated Polysaccharide Compositions

A polysaccharide composition of the invention can be an unformulated or formulated preparation. An “unformulated polysaccharide preparation” refers to a composition which comprises the polysaccharide but does not include a carrier or other excipient to enhance delivery or result in slow release. Compositions which include such carriers or excipients are referred to as “formulated”. Excipients and carriers which enhance delivery to and/or through a mucosal membrane are referred to herein as delivery enhancers. It was found that the polysaccharides of the invention can be delivered as unformulated compositions and still result in therapeutically effective levels of the polysaccharides being delivered by non-invasive routes.

The polysaccharides can also be generated to be in solid or liquid form. An example of a solid form is dry particles, e.g., dry particles for pulmonary delivery such as those described in PCT Publication Number 02/32406, the contents of which are incorporated herein by reference.

The polysaccharides of the invention may optionally be formulated in a pharmaceutically acceptable carrier. Such preparations may routinely contain pharmaceutically acceptable concentrations of salt, buffering agents, preservatives, compatible carriers, adjuvants, and optionally other therapeutic ingredients. The compositions may further be formulated into specific delivery devices. As described below, the polysaccharide may also be formulated based upon their intended route of delivery.

The compositions of the invention may be administered per se (neat) or in the form of a pharmaceutically acceptable salt. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2 mole % W/V); citric acid and a salt (1-3 mole % W/V); boric acid and a salt (0.5-2.5 mole % W/V); and phosphoric acid and a salt (0.8-2 mole % W/V). Suitable preservatives include benzalkonium chloride (0.003-0.03 mole % W/V); chlorobutanol (0.3-0.9 mole % W/V); parabens (0.01-0.25 mole % W/V) and thimerosal (0.004-0.02 mole % W/V).

The present invention provides pharmaceutical compositions, for medical use, which comprise sulfated polysaccharide preparations together with one or more pharmaceutically acceptable carriers and optionally other therapeutic ingredients. The term “pharmaceutically-acceptable carrier” as used herein means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with the sulfated polysaccharide of the present invention, and with each other, in a manner such that there is no interaction which would substantially impair the desired pharmaceutical efficiency.

Controlled release of polysaccharide can also be achieved with appropriate excipient materials that are biocompatible and biodegradable. These polymeric materials which effect slow release of the polysaccharide may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate.

Examples of preferred non-biodegradable polymers include ethylene vinyl acetate, poly(meth)acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

The polysaccharide compositions may include a single polysaccharide or multiple polysaccharides. Thus, the composition may include, for instance, only one polysaccharide, more than one polysaccharide but only one polysaccharide which has a diagnostic or therapeutic activity, or more than one polysaccharide having a diagnostic or therapeutic activity. In other aspects, the polysaccharide can be used as a carrier for an active agent, e.g., a therapeutic polypeptide. It has been found that in some situations the polysaccharides of the invention can be delivered regardless of the size of the particles to be delivered. Thus particles, e.g., particles which include a polysaccharide carrier and an active agent, can be greater than 5, 10, 15, 20, 25, 30 microns and still be administered in vivo in therapeutically effective amounts by certain routes of administration, e.g., pulmonary delivery.

Non-Invasive Routes of Administration

The polysaccharides of the invention can be delivered in vivo by various non-invasive routes of delivery. Non-invasive delivery refers to routes of delivery which do not require forced insertion of the polysaccharide through tissue, e.g., a layer of skin. Examples of non-invasive delivery methods which can be used with the polysaccharides of the invention include pulmonary (e.g., by inhalation or intranasal delivery), transdermal, and mucosal delivery (e.g., oral, buccal, sublingual, rectal or vaginal delivery). Invasive delivery methods, which require, e.g., forced pressure or an instrument to deliver through tissue, include intravenous, intramuscular and subcutaneous delivery.

Non-invasive delivery routes have several benefits including the ease of self administration by a subject, e.g., the polysaccharide composition can be in a dosage unit form. Dosage unit form as used herein refers to physically discrete units suited as unitary dosages for the subject to be treated; each unit containing a predetermined quantity of active compound calculated to produce the desired therapeutic effect. Examples of compositions which can be used for self administration include: metered amounts of a composition to be administered from an inhaler for pulmonary delivery; tablets having a prescribed dosage unit for oral administration; transdermal patches to deliver a dosage unit across the skin; and suppositories to deliver a desired dosage unit rectally or vaginally. The compositions can be included in a container, pack, or dispenser together with instructions for administration. These methods, as well as other methods used for non-invasive delivery, may also be used by health care professionals to administer the polysaccharides of the invention to a subject.

It is understood that the specific route of administration and dose level will depend upon a variety of factors including the activity of the specific compound employed, the age, body weight, general health, gender, and diet of the subject, the time of administration, the desired rate of absorption, bioavailability, the rate of excretion, any drug combination, and the location of desired therapeutic effect, e.g., local or systemic effect. A local therapeutic effect refers to a biologic effect that occurs at the tissue where the polysaccharide is delivered. For instance, when the polysaccharide is a heparin, it may be desirable to deliver the polysaccharide the lung to produce a local effect for the treatment of, e.g., a respiratory disease. Other non-invasive routes which can be used to deliver a local therapeutic effect include intranasal, buccal, rectal and vaginal delivery. A systemic effect refers to a biologic effect that occurs outside of the tissue/organ where the composition is delivered, e.g., the biological effect occurs in the blood.

Pulmonary Delivery

It was found that identification of chemical properties of polysaccharides, e.g., based upon their chemical signature, can be used to generate enhanced formulations for delivering compounds by a pulmonary route, e.g., by inhalation through the mouth or nasal passage. For example, by neutralizing the net charge of a polysaccharide such as an HLGAG, the ability of the polysaccharide to permeate a lipid membrane, e.g., an alveolar membrane, of the lung may be enhanced.

The polysaccharides of the invention can be administered by inhalation to pulmonary tissue. The term “pulmonary tissue” as used herein refers to any tissue of the respiratory tract and includes both the upper and lower respiratory tract, except where otherwise indicated.

For administration by inhalation, the polysaccharides are delivered in the form of an aerosol spray from pressured container or dispenser which contains a suitable propellant or a nebulizer. The polysaccharides may be in the form of a dry particle or as a liquid. Preferably, the polysaccharides are delivered to pulmonary tissue as a dry particle. Polysaccharide particles can be prepared, e.g., by drying an aqueous polysaccharide solution with a charge neutralizing agent and then creating particles from the dried powder or by drying an aqueous polysaccharide solution in an organic modifier and then creating particles from the dried powder.

The polysaccharides may be conveniently delivered in the form of an aerosol spray presentation from pressurized packs or a nebulizer, with the use of a suitable propellant, e.g., dichlorodifluoromethane, trichlorofluoromethane, dielilorotetrafluoroctliane, carbon dioxide or other suitable gas. In the case of a pressurized aerosol, the dosage unit may be determined by providing a valve to deliver a metered amount. Capsules and cartridges for use in an inhaler or insufflator may be formulated containing a powder mix of the polysaccharide and a suitable powder base such as lactose or starch, if the particle is a formulated particle. In addition to the formulated or unformulated polysaccharides, e.g., polysaccharide particles, administered, other materials such as 100% DPPC or other surfactants can be mixed with the polysaccharide to promote the delivery and dispersion of formulated or unformulated polysaccharides. Methods of preparing dry polysaccharide particles are described, for example, in PCT Publication WO 02/32406.

The polysaccharide, e.g., dry aerosol particles, when administered are rapidly absorbed and can produce a rapid local or systemic therapeutic result. It has been discovered that the peak activity of the delivered polysaccharide can be achieved within 3 hours and preferably within two hours. In some embodiments, the peak activity can be achieved even more quickly, e.g., within one half hour or even within ten minutes. Based on the rapid absorption rates of the polysaccharides of the invention by pulmonary delivery, inhaled heparin allows a rapid anticoagulation/antithrombosis state in the blood which cannot be achieved with subcutaneous administration of LMWHs. The rapid absorption of heparin after inhalation can be combined with subsequent subcutaneous administration of LMWHs to improve the efficiency of a desired activity, e.g., antithrombotic/anticoagulation treatment. Alternatively, heparin formulated for longer biological half-life can be used as an alternative to subcutaneous administration of LMWHs. Similar regimens can also be adopted for use of heparin in cerebral vascular diseases such as stroke, which require immediate early intervention. These and other therapeutic uses are described in more detail below.

In one embodiment, the polysaccharide is delivered in an amount such that 5% of the polysaccharide is delivered to the lower respiratory tract or the deep lung. Deep lung has the richest capillary network found in any organ in the human body, and the respiratory membrane separating capillary lumen from the alveolar air space is very thin (≦6 μm) and extremely permissible. Thus, it is desirable to deliver to that portion of the lung. In addition, the liquid layer lining the alveolar surface is rich in lung surfactants. In other embodiments, at least 2%, 3%, 5%, 10%, 20%, 30%, 40%, 50%, 60%, 70%, or 80% of the polysaccharide composition is delivered to the lower respiratory tract or to the deep lung. Delivery to either or both of these tissues results in efficient absorption of the polysaccharide and high bioavailability. In one embodiment, the polysaccharide is provided in a metered dose using, e.g., an inhaler or nebulizer. Preferably, the polysaccharide is delivered in a dosage unit form of at least about 15 mg/puff, 20 mg/puff, 25 mg/puff, 30 mg/puff, 35 mg/puff, 40 mg/puff, 45 mg/puff, 50 mg/puff, 55 mg/puff, 60 mg/puff, 70 mg/puff, 80 mg/puff, 90 mg/puff, 100 mg/puff or more.

The percent bioavailability can be calculated as follows: the percent bioavailability (AUCₙₒₙ₋ᵢₙᵥₐₛᵢᵥₑ/AUC_{i.v or s.c.})×(dose_{i.v. or s.c.}/doseₙₒₙ₋ᵢₙᵥₐₛᵢᵥₑ)×100.

Although not necessary to achieve the desired levels of delivery, delivery enhancers such as surfactants can be used to further enhance pulmonary delivery. A “surfactant” as used herein refers to a compound having a hydrophilic and lipophilic moiety, which promotes absorption of a drug by interacting with an interface between two immiscible phases. Surfactants are useful in the dry particles for several reasons, e.g., reduction of particle agglomeration, reduction of macrophage phagocytosis, etc. When coupled with lung surfactant, a more efficient absorption of polysaccharides can be achieved because surfactants, such as DPPC, will greatly facilitate diffusion of polysaccharides, such as heparin across the membrane surface of the alveoli by disguising the hydrophilic, charged groups of the heparin polymer. Surfactants are well known in the art and include but are not limited to phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9-; auryl ether; palmitic acid; oleic acid; sorbitan trioleate (Span 85); glycocholate; surfactin; poloxomer; sorbitan fatty acid ester; sorbitan trioleate; tyloxapol; phospholipids.

Mucosal Delivery

It was found that identification of chemical properties of polysaccharides, e.g., based upon their chemical signature, can be used to generate enhanced compositions for mucosal delivery of compounds. The terms “mucosa” and “mucosal” refer to mucous tissue, epithelium, lamina propria and the layer of smooth muscle in the digestive and reproductive tract. Methods of mucosal delivery include oral, buccal, sublingual, rectal and vaginal delivery.

For oral administration, the compounds (e.g., LMWH preparations) can be formulated by combining the active compound(s) with a pharmaceutically acceptable carrier. Such carriers allow the compounds of the invention to be formulated as tablets, pills, dragees, capsules, liquids, gels, syrups, slurries, suspensions and the like, for oral ingestion by the subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients include fillers such as sugars, (e.g., lactose, sucrose, mannitol or sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP)). If desired, disintegrating agents may be added, such as cross-linked polyvinylpyrrolidone, agar or alginate may also be formulated in saline buffers for neutralizing internal acid conditions or may be administered without any carriers.

Dragee cores are provided with suitable coatings. For this purposes, concentrated sugar solutions can be used, which may optionally contain gum arabic, talc, polyvinylpyrrolidine, carbopol gell, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain active ingredients in admixture with fillers such as lactose, binders such as starches, and/or lubricants such as talc or magnesium stearate and, optionally stabilizers. In soft capsules, the active compounds may be dissolved or suspended in suitable liquids such as fatty oils, liquid paraffin, or liquid polyethylene glycols. In addition, stabilizers may be added. Microspheres formulated for oral administration may also be used. Such microspheres are known in the art. It is advantageous to formulate oral compositions in appropriate dosage units.

When a polysaccharide composition of the invention is being delivered orally such that the polysaccharide must pass through a membrane, e.g., the intestinal mucosa, to achieve systemic delivery, delivery enhancers such as penetrants appropriate to the barrier to be permeated can be used in the formulation. Such penetrants are generally known in the art, and include, for example, detergents, bile salts, and fusidic acid derivatives.

Transmucosal administration can be accomplished through rectal delivery by, e.g., the use of suppositories (e.g., with conventional suppository bases such as cocoa butter and other glycerides) or retention enemas for rectal delivery.

For buccal administration, the compositions may take the form of tablets, lozenges and mouth rinses formulated in conventional manner.

As demonstrated herein, sulfated polysaccharides are stable under the acidic and enzymatic environment of the gastrointestinal tract, including the harsh conditions of the stomach. By neutralizing and/or reducing the mass of sulfonated polysaccharides such as LMWH, it was found that these polysaccharides are capable of passing through the intestinal mucosa and entering the blood stream. As demonstrated in the Examples, LMWHs have been prepared having a reduced mass and net charge as compared to enoxaparin. These LMWH were shown to be absorbed through the intestinal mucosa at therapeutically acceptable levels with an acceptable absorption half-life. The term “therapeutically acceptable levels” refers to the delivery of a polysaccharide composition, for local or systemic effect, at a level sufficient to result in the occurrence of a desired activity in a cell or subject. For example, it is generally accepted that a minimum therapeutic level of HLGAG for producing therapeutic anticoagulant effect is approximately 0.3 IU/ml anti-factor Xa activity. Accordingly, in one aspect when the polysaccharide is an HLGAG, and the desired activity is anti-Xa activity, the HLGAG (e.g., LMWH) is absorbed by the gastrointestinal tract at a level of at least about 0.2 IU/ml, preferably, at least about 0.25, 0.30, 0.35, 0.4 IU/ml or higher (e.g., 0.5 IU/ml, 1.0 IU/ml) over a period of 1 to 5, preferably 2, 3, or 4 hours after administration. Preferably, the absorption rate of the polysaccharides, e.g., LMWH, is within 1 to 5 hours, preferably 2, 3 or 4 hours after administration.

A “therapeutically effective amount” refers to an amount of the polysaccharide which is effective, upon single or multiple dose administration to a subject, in treating, alleviating, relieving or improving a symptom of a subject as described herein beyond that expected in the absence of such treatment.

Methods for Monitoring Non-Invasive Delivery

The amount of polysaccharide delivered can be determined using routine methods. For instance to determine delivery by inhalation, in a test system, lavage of animal lungs at indicated time intervals after inhalation can be used to determine the amount of heparin delivered to the lower respiratory tract. Similar tests can be done to determine levels of polysaccharide in, e.g., the intestinal mucosa, at various points after oral delivery. This data can be correlated to that amount which would occur in humans or animals being treated. Alternatively, a label, such as a radioactive or fluorescent label can be attached to the polysaccharide and used to determine the distribution of the delivered polysaccharide. The amount of polysaccharide delivered to a desired tissue can also be determined as the amount of therapeutic effect resulting from the presence of the polysaccharide in that tissue or in the region where the biological activity is occurring, e.g., the blood, or the blood plasma concentration of the polysaccharide. The type of parameter used to assess the effectiveness of the delivery will vary depending on a variety of factors including the type of subject, the type of equipment available, and the disorder being treated or prevented. The peak plasma concentration of a polysaccharide can be determined by measuring the level of polysaccharide present in the blood over time and determining when the peak level of concentration is reached. The amount of a therapeutic effect or a peak plasma activity can be identified using routine assays. The type of these effects will depend on the therapeutic parameter being assessed. For instance, if the polysaccharide is administered in order to prevent coagulation, the amount of inhibition of factor Xa activity can be assessed. It is known that a minimum therapeutic level of heparin-like glycosaminoglycan for producing therapeutic anticoagulant effect is approximately 0.3 IU/ml anti-factor Xa activity. HLGAGs are also useful for inhibiting enzymatic activity, such as human leukocyte elastase. In general, the IC50 of HLGAGs on human leukocyte elastase ranges from 1 ng/ml to 50 microgram/ml. Alternatively, the A₁ values of HLGAGs ranges from 10 nm to 10 μM. In other instances, when the polysaccharide is a polysaccharide isolated from phellinus linteus, the biological activity which can be assessed includes both cell-mediated immunity and humoral immunity. Thus, the level of cell-mediated immunity or antibody production may be measured in order to characterize the therapeutic effect of peak biological activity of these compounds. Other assays are well known to those of ordinary skill in the art for various polysaccharides.

Therapeutic Uses

The compositions may be administered to a subject. As used herein, a subject is a vertebrate such as a human, non-human primate, cow, horse, pig, sheep, goat, dog, cat, or rodent. The subject can be, e.g., an experimental animal, a veterinary animal, or a human subject.

HLGAGs have many therapeutic utilities. The HLGAG compositions can be used for the treatment of any type of condition in which HLGAG therapy is useful. Thus, the products and methods are useful in a variety of in vitro, in vivo and ex vivo methods in which HLGAG therapies are useful. For instance, it is known that HLGAG compositions are useful for preventing and treating dementia, such as Alzheimer's disease, coagulation, angiogenesis, thrombotic disorders, cardiovascular disease, vascular conditions, atherosclerosis, respiratory disorders, circulatory shock and related disorders, as well as inhibiting cancer cell growth and metastasis. Each of these disorders is well-known in the art and is described, for instance, in Harrison 's Principles of Internal Medicine (McGraw Hill, Inc., New York), which is incorporated by reference. The use of HLGAG compositions in various therapeutic methods is described and summarized in Huang, J. and Shimamura, A., Coagulation Disorders, 12, 1251-1281 (1998).

Thus, the HLGAG preparations are useful for treating or preventing disorders associated with coagulation. When an imbalance in the coagulation pathway shifts towards excessive coagulation, the result is the development of thrombotic tendencies, which are often manifested as heart attacks, strokes, deep venous thrombosis, acute coronary syndromes (ACS) such as unstable angina, and myocardial infarcts. A “disease associated with coagulation” as used herein refers to a condition characterized by local inflammation which can result from an interruption or reduction in the blood supply to a tissue which may occur, for instance, as a result of blockage of a blood vessel responsible for supplying blood to the tissue such as is seen for myocardial or cerebral infarction or peripheral vascular disease, or as a result of emboli formation associated with conditions such as atrial fibrillation or deep venous thrombosis. Coagulation disorders include, but are not limited to, cardiovascular disease and vascular conditions such as cerebral ischemia. It is particularly useful to treat disorders such as myocardial infarction and ACS with, e.g., a polysaccharide by pulmonary delivery because of the fast absorption and action of this delivery system.

The methods are useful for treating cardiovascular disease. Cardiovascular diseases include, but are not limited to, acute myocardial infarction, ACS, e.g., unstable angina, and atrial fibrillation. Myocardial infarction is a disease state which sometimes occurs with an abrupt decrease in coronary blood flow that follows a thrombotic occlusion of a coronary artery previously narrowed by atherosclerosis. Such injury may be produced or facilitated by factors such as cigarette smoking, hypertension, and lipid accumulation. Acute angina is due to transient myocardial ischemia. This disorder is usually associated with a heaviness, pressure, squeezing, smothering, or choking feeling below the sternum. Episodes are usually caused by exertion or emotion, but can occur at rest.

Atrial fibrillation is a common form of arrhythmia generally arising as a result of emotional stress or following surgery, exercise, or acute alcoholic intoxication. Persistent forms of atrial fibrillation generally occur in patients with cardiovascular disease. Atrial fibrillation is characterized by disorganized atrial activity without discrete P waves on the surface ECG. This disorganized activity can lead to improper blood flow in the atrium and thrombus formation. These thrombi can embolize, resulting in cerebral ischemia and other disorders.

Persons undergoing surgery, anesthesia and extended periods of bed rest or other inactivity are often susceptible to a condition known as deep venous thrombosis, or DVT, which is a clotting of venous blood in the lower extremities and/or pelvis. This clotting occurs due to the absence of muscular activity in the lower extremities required to pump the venous blood (stasis), local vascular injury or a hypercoaguble state. The condition can be life-threatening if a blood clot migrates to the lung, resulting in a “pulmonary embolus” or otherwise interferes with cardiovascular circulation. One method of treatment involves administration of an anti-coagulant.

The compounds can be used for the treatment of cardiovascular disorders alone or in combination with other therapeutic agents for reducing the risk of a cardiovascular disease or for treating the cardiovascular disease. Other therapeutic agents include, but are not limited to, anti-inflammatory agents, anti-thrombotic agents, anti-platelet agents, fibrinolytic agents, lipid reducing agents, direct thrombin inhibitors, anti-Xa inhibitors, anti-IIa inhibitors, glycoprotein IIb/IIIa receptor inhibitors and direct thrombin inhibitors such as hirudin, hirugen, Angiomax, agatroban, PPACK, thrombin aptamers.

The HLGAG preparations are also useful for treating vascular conditions. Vascular conditions include, but are not limited to, disorders such as deep venous thrombosis, peripheral vascular disease, cerebral ischemia, including stroke, and pulmonary embolism. A cerebral ischemic attack or cerebral ischemia is a form of ischemic condition in which the blood supply to the brain is blocked. This interruption or reduction in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue.

The methods are useful for treating cerebral ischemia. Cerebral ischemia may result in either transient or permanent deficits and the seriousness of the neurological damage in a patient who has experienced cerebral ischemia depends on the intensity and duration of the ischemic event. A transient ischemic attack is one in which the blood flow to the brain is interrupted only briefly and causes temporary neurological deficits, which often are clear in less than 24 hours. Symptoms of TIA include numbness or weakness of face or limbs, loss of the ability to speak clearly and/or to understand the speech of others, a loss of vision or dimness of vision, and a feeling of dizziness. Permanent cerebral ischemic attacks, also called stroke, are caused by a longer interruption or reduction in blood flow to the brain resulting from either a thrombus or embolism. A stroke causes a loss of neurons typically resulting in a neurologic deficit that may improve but that does not entirely resolve.

Thromboembolic stroke is due to the occlusion of an extracranial or intracranial blood vessel by a thrombus or embolus. Because it is often difficult to discern whether a stroke is caused by a thrombosis or an embolism, the term “thromboembolism” is used to cover strokes caused by either of these mechanisms.

The rapid absorption of HLGAGs, such as UFH or LMWH, after inhalation can be very valuable in the treatment of venous thromboembolism. Intravenous administration of UFH has been used widely for treatment of venous thromboembolism in combination with oral warfarin. Due to the improved efficacy and reduced risks, however, LMWHs have been increasingly used as an alternative to intravenous UFH in treatment of venous thromboembolism. It has been established that efficacy of heparin therapy depends on achieving critical therapeutic levels (e.g., of values of anti-factor Xa or anti-factor IIa activity) within the first 24 hours of treatment. Intrapulmonary delivery of heparin particles to achieve rapid therapeutic levels of heparin in the early stage of thromboembolism, could also be combined with other routes of administration of LMWHs or heparin for prolonged antithrombotic/anticoagulant effect such as oral administration.

The methods are also directed to the treatment of acute thromboembolic stroke using HLGAGs. An acute stroke is a medical syndrome involving neurological injury resulting from an ischemic event, which is an interruption or reduction in the blood supply to the brain.

An effective amount of a HLGAG preparation alone or in combination with another therapeutic for the treatment of stroke is that amount sufficient to reduce in vivo brain injury resulting from the stroke. A reduction of brain injury is any prevention of injury to the brain which otherwise would have occurred in a subject experiencing a thromboembolic stroke absent the treatment described herein. Several physiological parameters may be used to assess reduction of brain injury, including smaller infarct size, improved regional cerebral blood flow, and decreased intracranial pressure, for example, as compared to pretreatment patient parameters, untreated stroke patients or stroke patients treated with thrombolytic agents alone.

The pharmaceutical HLGAG preparation may be used alone or in combination with a therapeutic agent for treating a disease associated with coagulation. Examples of therapeutics useful in the treatment of diseases associated with coagulation include anticoagulation agents, antiplatelet agents, and thrombolytic agents.

Anticoagulation agents prevent the coagulation of blood components and thus prevent clot formation. Anticoagulants include, but are not limited to, warfarin, Coumadin, dicumarol, phenprocoumon, acenocoumarol, ethyl biscoumacetate, and indandione derivatives. “Direct thrombin inhibitors” include hirudin, hirugen, Angiomax, agatroban, PPACK, thrombin aptamers. Antiplatelet agents inhibit platelet aggregation and are often used to prevent thromboembolic stroke in patients who have experienced a transient ischemic attack or stroke. Thrombolytic agents lyse clots which cause the thromboembolic stroke. Thrombolytic agents have been used in the treatment of acute venous thromboembolism and pulmonary emboli and are well known in the art (e.g. see Hennekens et al, J Am Coll Cardiol; v. 25 (7 supp), p. 18S-22S (1995); Holmes, et al, J Am Coll Cardiol; v.25 (7 suppl), p. 10S-17S(1995)).

Pulmonary embolism as used herein refers to a disorder associated with the entrapment of a blood clot in the lumen of a pulmonary artery, causing severe respiratory dysfunction. Pulmonary emboli often originate in the veins of the lower extremities where clots form in the deep leg veins and then travel to lungs via the venous circulation. Thus, pulmonary embolism often arises as a complication of deep venous thrombosis in the lower extremity veins. Symptoms of pulmonary embolism include acute onset of shortness of breath, chest pain (worse with breathing), and rapid heart rate and respiratory rate. Some individuals may experience haemoptysis.

The products and methods are also useful for treating or preventing atherosclerosis. Heparin has been shown to be beneficial in prevention of atherosclerosis in various experimental models. Due to the more direct access to the endothelium of the vascular system, inhaled heparin can be useful in prevention of atherosclerosis. Atherosclerosis is one form of arteriosclerosis that is believed to be the cause of most coronary artery disease, aortic aneurysm and atrial disease of the lower extremities, as well as contributing to cerebrovascular disease.

Due to its fast absorption and variable elimination rate, HLGAG with or without excipients can be used as an alternative for the intravenous heparin for surgical and dialysis procedures. For example, HLGAG particles can be inhaled prior to surgery by volunteer inhalation or passively inhaled via trachea tube during the anesthesia prior to or during the surgery. Surgical patients, especially those over the age of 40 years have an increased risk of developing deep venous thrombosis. Thus, the use of HLGAG particles for preventing the development of thrombosis associated with surgical procedures is contemplated. In addition to general surgical procedures such as percutaneous intervention (e.g., percutaneous coronary intervention (PCI)), PCTA, stents and other similar approaches, hip or knee replacement, cardiac-pulmonary by-pass surgery, coronary revascularization surgery, orthopedic surgery, and prosthesis replacement surgery, the methods are also useful in subjects undergoing a tissue or organ transplantation procedure or treatment for fractures such as hip fractures.

In addition, pulmonary inhalation of heparin is valuable in treatment of respiratory diseases such as cystic fibrosis, asthma, allergy, emphysema, adult respiratory distress syndrome (ARDS), lung reperfusion injury, and ischemia-reperfusion injury of the lung, kidney, heart, and gut, and lung tumor growth and metastasis.

Cystic fibrosis is a chronic progressive disease affecting the respiratory system. One serious consequence of cystic fibrosis is Pseudomonas aeruginosa lung infection, which by itself accounts for almost 90% of the morbidity and mortality in cystic fibrosis. Therapeutics for treating cystic fibrosis include antimicrobials for treating the pathogenic infection.

Heparin is also a well established inhibitor of elastase and tumor growth and metastasis. The aerosolized heparin particles are capable of inhibiting elastase induced lung injury in an acute lung emphysema model. Asthma is a disorder of the respiratory system characterized by inflammation, narrowing of the airways and increased reactivity of the airways to inhaled agents. Asthma is frequently, although not exclusively, associated with atopic or allergic symptoms. Asthma may also include exercise induced asthma, bronchoconstrictive response to bronchostimulants, delayed-type hypersensitivity, auto immune encephalomyclitis and related disorders. Allergies are generally caused by IgE antibody generation against allergens. Emphysema is a distention of the air spaces distal to the terminal bronchiole with destruction of alveolar septa. Emphysema arises out of elastase induced lung injury. Heparin is capable of inhibiting this elastase induced injury. Adult respiratory distress syndrome is a term which encompasses many acute defuse infiltrative lung lesions of diverse ideologies which are accompanied by severe atrial hypoxemia. One of the most frequent causes of ARDS is sepsis. Inflammatory diseases include but are not limited to autoimmune diseases and atopic disorders. Other types of inflammatory diseases which are treatable with HLGAGs are refractory ulcerative colitis, Chrohn's disease, multiple sclerosis, autoimmune disease, non-specific ulcerative colitis and interstitial cystitis.

In one embodiment, the HLGAG preparations are used for inhibiting angiogenesis. An effective amount for inhibiting angiogenesis of the HLGAG preparation is administered to a subject in need of treatment thereof. Angiogenesis as used herein is the inappropriate formation of new blood vessels. “Angiogenesis” often occurs in tumors when endothelial cells secrete a group of growth factors that are mitogenic for endothelium causing the elongation and proliferation of endothelial cells which results in the generation of new blood vessels. Several of the angiogenic mitogens are heparin binding peptides which are related to endothelial cell growth factors. The inhibition of angiogenesis can cause tumor regression in animal models, suggesting a use as a therapeutic anticancer agent. An effective amount for inhibiting angiogenesis is an amount of HLGAG preparation which is sufficient to diminish the number of blood vessels growing into a tumor. This amount can be assessed in an animal model of tumors and angiogenesis, many of which are known in the art. Angiogenic disorders include, but are not limited to, neovascular disorders of the eye, osteoporosis, psoriasis, arthritis, cancer and cardiovascular disorders.

The HLGAG preparations are also useful for inhibiting neovascularization associated with eye disease. In another embodiment, the HLGAG preparation is administered to treat psoriasis. Psoriasis is a common dermatologic disease caused by chronic inflammation.

HLGAG containing compositions, may also inhibit cancer cell growth and metastasis. Thus the methods are useful for treating and/or preventing tumor cell proliferation or metastasis in a subject. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; leukemias, lymphomas; liver cancer; lung cancer (e.g. small cell and non-small cell); melanoma; neuroblastomas; oral cancer; ovarian cancer; pancreatic cancer; prostate cancer; rectal cancer; sarcomas; skin cancer; testicular cancer; thyroid cancer; and renal cancer, as well as other carcinomas and sarcomas.

A subject in need of cancer treatment may be a subject who has a high probability of developing cancer. These subjects include, for instance, subjects having a genetic abnormality, the presence of which has been demonstrated to have a correlative relation to a higher likelihood of developing a cancer and subjects exposed to cancer-causing agents such as tobacco, asbestos, or other chemical toxins, or a subject who has previously been treated for cancer and is in apparent remission.

When administered to a patient undergoing cancer treatment, the polysaccharide particles may be administered in cocktails containing other anti-cancer agents. The polysaccharide compositions may also be administered in cocktails containing agents that treat the side-effects of radiation therapy, such as anti-emetics, radiation protectants, etc.

The terms “prevent” and “preventing” as used herein refer to inhibiting completely or partially the biological effect, e.g., inflammation, angiogenesis or proliferation or metastasis of a cancer or tumor cell, as well as inhibiting any increase in the biological effect.

Effective amounts of the polysaccharide particles are administered to subjects in need of such treatment. Effective amounts are those amounts which will result in the desired biological effect. The desired biological effect will depend on factors such as the type of polysaccharide particles (i.e. the type of active agent) being administered and the type of disease being prevented or treated. For instance, when the active agent in a polysaccharide composition is an HLGAG, the biological effect may be a reduction in cellular proliferation or metastasis, a reduction in inflammation, an inhibition of elastase, prevention of respiratory disease, or prevention of coagulation without causing other medically unacceptable side effects. Such amounts can be determined with no more than routine experimentation. It is believed that doses ranging from 1 nanogram/kilogram to 100 milligrams/kilogram, depending upon the mode of administration, will be effective. The effective percentage of polysaccharide particles may be determined with no more than routine experimentation. The absolute amount will depend upon a variety of factors (including whether the administration is in conjunction with other methods of treatment, the number of doses and individual patient parameters including age, physical condition, size and weight) and can be determined with routine experimentation. It is preferred generally that a maximum dose be used, that is, the highest safe dose according to sound medical judgment.

One advantage of using non-invasive delivery is the convenience of administration, which allows self-administration on an outpatient basis. This will enable a faster initiation of treatment with heparin. Thus a subject may keep a device, such as an inhaler, or tablets, patches or suppositories for self administering the polysaccharide when necessary. This is particularly useful for HLGAGs, which in some cases require rapid administration. The polysaccharides may also be administered by a health care professional, e.g. with the use of a tracheal tube or by intraduodenl delivery. Such methods are well known in the art.

In addition to HLGAGs, other polysaccharides have a diverse array of therapeutic utilities. Chondroitin Sulfate has been used in a complex with cisplatin to reduce the nephrotoxity of cisplatin during chemotherapy. Zhang J S, Imai T, Otagiri M. Arch Toxicol 2000 August;74(6):300-7). Hyaluronic acid and derivatives thereof have been shown to be a pharmacological class of slow acting drugs for the treatment of osteoarthritis. Watterson J R, Esdaile J M, J Am Acad Orthop Surg 2000 October;8(5):277-284). Chitin, which is a non-sulfated polysaccharide, can be sulfated chemically to produce a modified polysaccharide, e.g., 6-0 sulfated carboxymethyl chitin which is capable of inhibiting lung metastasis of melanoma. Murata J, Saiki I, Makabe T, Tsuta Y, Tokura S, Azuma I, Cancer Res 1991 Jan. 1; 51(1):22-6. Nishiyama Y, Yoshikawa T, Kurita K, Hojo K, Kamada H, Tsutsumi Y, Mayumi T, Kawasaki K. Chem Pharm Bull (Tokyo) 1999 March;47(3):451-3. Polysaccharide isolated from phellinus linteus are also useful for treating and preventing melanoma, especially when administered in combination with adriamycin. Han S B, Lee C W, Jeon Y J, Hong N D, Yoo I D, Yang K H, Kim H M. 1999 February;41(2):157-64.). Calcium spirulan, isolated from a blue-green algae, spirulina platensis, is a sulfated polysaccharide that is mainly composed of rhamnose and has been demonstrated to inhibit tumor invasion and metastasis. Hayakawa Y, Hayashi T, Lee J B, Ozawa T, Sakuragawa N. J Biol Chem 2000 Apr. 14;275(15):11379-82). Heparin mimetics such as oligosaccharides and pentasaccharides are useful for preventing coagulation and thrombosis. Other glycomimetics have been used for prevention of coagulation as well as treatment of inflammation, cancer and other immunologic disorders. (Barchi, J. J., Curr. Pharm. Des., 2000, 6(4):485-501) Synthetically derived sulfated polysaccharides, such as laminarin are useful for inhibiting heparinase and thus for inhibiting inflammation, tumor progression, etc. (Marchetti, D. et al., Cancer Res., 2000, 60:4767-70). PI-88 is a mixture of highly sulfated oligosaccharides derived from the sulfation of phosphomannum which is purified from a high molecular weight core produced by fermentation of the yeast pichia holstii. The main constituent is a pentamannose, however, small amounts of tetrasaccharide and minor amount of hexasaccharide are also present. P188 is currently undergoing clinical trials for its anticoagulant/antithrombotic properties. P188 is also a potent inhibitor of heparan sulfate binding and inhibits heparinase enzymatic activity. (Parish, C. R., et al., Cancer Res., 1999, 59:3433-41).

Other polysaccharides which are useful are polysaccharide vaccine antigens. These antigens can be delivered alone or in combination with standard vaccine adjuvants for the purpose of stimulating an immune response. The polysaccharide antigen is a polysaccharide which is capable of eliciting an immune response against a microorganism in a host. These include, but are not limited to, capsular polysaccharides, lipopolysaccharides and other subcapsular (surface) polysaccharides. Examples of capsular polysaccharides include those isolated from Haemophilus influenzae, Neisseria meningitidis, Streptococcus pneumoniae, Streptococcus agalactiae, Salmonella typhi, Escherichia coli, and Staphylococcus aureus. Examples of lipopolysaccharides are those isolated from Neisseria meningitidis, Escherichia coli, Salmonella typhi, and Pseudomonas aeruginosa. Examples of other subcapsular polysaccharides are the common polysaccharide antigen (c-substance) of Group A, B and C Streptococci and the common polysaccharide antigen (c-substance) of Streptococcus pneumoniae. The immunology of polysaccharide vaccines has been reviewed by Jennings et al, “The Polysaccharides” (Editor; G. O. Aspinall), Volume 1, 291-329 (1982). See also “Carbohydrate Chemistry,” ed. by John F. Kennedy, Clarendon Press, Oxford, 1988; “The Carbohydrates, Chemistry and Biochemistry,” ed. by W. Pigman and D. Horton, Academic Press, Inc., 1970; and “Chitin, Chitosan, and Related Enzymes,” ed. by John P. Zikakis, Academic Press, Inc., 1984.

The polysaccharides also include vaccine adjuvants. These polysaccharide adjuvants can be delivered alone or in combination with standard vaccine antigens for the purpose of stimulating an immune response.

The polysaccharide composition, in some embodiments, may include compounds other than polysaccharides, as long as the composition includes at least one polysaccharide. These include, for instance, but are not limited to, active agents such as proteins, nucleic acids, small organic or inorganic molecules, carriers that do not have slow release properties, preservatives, etc. Thus, the methods relate to non-invasive administration of active agents. An active agent as used herein is any compound which has a diagnostic, prophylactic, or therapeutic effect in a biological organism. The active agents may optionally be proteins, peptides, antibodies, polysaccharides, nucleic acids (e.g., RNA, DNA, PNA, multiplexes of them (e.g.: triplex)), saccharides, glycoproteins, amino acids, viruses, heterogeneous mixtures of macromolecules (e.g., a natural product extract) and hybrid macromolecules (e.g., protein/nucleic acid hybrids, albumin conjugated proteins, drugs with linkers inorganic molecules, organic molecules, or combinations thereof.

A bioactive agent is any compound which has a prophylactic or therapeutic effect in a biological organism. In some embodiments the bioactive agent is any of the drugs described above or one or more of the following agents: adrenergic agent; adrenocortical steroid; adrenocortical suppressant; agents for treating cognition, antiplatelets, aldosterone antagonist; amino acid; anabolic; analeptic; analgesic; anesthetic; anorectic; anti-acne agent; anti-adrenergic; anti-allergic; anti-Alzheimer's, anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; antiepileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; anxiolytics, appetite suppressant; blood glucose regulator; bone resorption inhibitor; bronchodilator; cardiovascular agent; cholinergic; COX1 inhibitors, COX2 inhibitors, direct thrombin inhibitors, depressant; diagnostic aid; diuretic; dopaminergic agent; estrogen receptor agonist; fibrinolytic; fluorescent agent; free oxygen radical scavenger; gastrointestinal motility effector; glucocorticoid; GPIIbIIIa antagonists, hair growth stimulant; hemostatic; histamine H2 receptor antagonists; hormone; human growth hormone, hypocholesterolemic; hypoglycemic; hypolipidemic; hypnotics, hypotensive; imaging agent; immunological agents such as immunizing agents, immunomodulators, immunoregulators, immunostimulants, and immunosuppressants; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; proton pump inhibitors, psychotropic; radioactive agent; scabicide; sclerosing agent; sedative; sedative-hypnotic; selective adenosine A1 antagonist; serotonin antagonist; serotonin inhibitor; serotonin receptor antagonist; statins, steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor.

In some embodiments the polysaccharide and the active agent are distinct and in other embodiments they are the same. Thus, in some formulations, the active agent is the polysaccharide of the polysaccharide composition and no further active agent is incorporated. In other formulations, the polysaccharide contains a polysaccharide and an active agent that is different than the polysaccharide. A “polysaccharide and the active agent are distinct” when they are different chemical entities. A polysaccharide may be distinct from the active agent if the active agent is a polysaccharide as long as the active agent is not the identical polysaccharide. For instance, the polysaccharide preparation may be LMWH and the active agent may be UFH, hyaluronic acid etc. Alternatively, the polysaccharide may not be an active agent and the polysaccharide composition may include an additional active agent.

The following description of experiments performed is exemplary and non-limiting to the scope of the claimed invention.

### EXAMPLES

### Example 1

### Synthesis of Enoxaparin-Derived LMWH Compounds

Step 1: 100 mg of enoxaparin was dissolved in 10 ml of water to get 10 mg/ml concentration. 100 mg NaCl was added to this solution. The pH of the solution was adjusted to 6.7.5 ml 200 Proof ethanol was added to this mixture. The solution was maintained at 4° C. for 24 hours. The residue (MLP) that is precipitated is removed by centrifugation at 4000 RPM for 15 minutes. 20 ml ethanol was added to the supernatant, and the mixture maintained at 4° C. for 24 hours. The precipitate formed at the end of 24 h (MLP) is separated by centrifugation at 4000 RPM for 15 min. It is lyophilized overnight to give 60 mg dry powder of MLS.

Step 2: 100 mg MLS was dissolved in 10 ml of 50 mM Calcium Acetate buffer, pH 6.7. An enzyme cocktail consisting of 10 mg Heparinase II and 1 mg of Heparinase III was added to this mixture, and the solution was maintained at 37° C. for 4 hours. The precipitate formed at the end of 2 hours was removed by centrifugation at 4000 RPM for 15 minutes. The supernatant of digested MLS was desalted in a size exclusion chromatography column.

Step 3: 100 mg MLS digested by the method explained above was loaded on a lm long, 10 cm diameter P10 size exclusion column. 500 mM Ammonium Acetate buffer was used as the running buffer. The eluent was tracked by absorption at UV 232 nM. 3 ml peaks were collected after the initial void volume. The peaks that gave absorption of more than 0.1 unit were collected. They were divided into 10 equal fractions. The different fractions were then lyophilized from water to get rid of ammonium bicarbonate salt. They were then assayed for the building blocks and functional characteristics (anti-Xa, and anti-IIa activity) by the assays described. Characteristics of Fraction 3 and Fraction 7 (named as M108, and M405) are listed in Tables 1 and 2 below.

### Example 2

### Synthesis of UFH-Derived LMWH Compounds

Step 1: 100 mg of UFH was dissolved in 10 ml of water to get 10 mg/ml concentration. 100 mg NaCl was added to this solution. The pH of the solution was adjusted to 6.7.3 ml 200 Proof ethanol was added to this mixture. The solution was maintained at 4° C. for 12 hours. The residue (MUP) that is precipitated is removed by centrifugation at 4000 RPM for 15 minutes. 10 ml ethanol was added to the supernatant, and the mixture maintained at 4° C. for 24 hours. The precipitate formed at the end of 24 hours (MUS) is separated by centrifugation at 4000 RPM for 15 minutes. It is lyophilized overnight to give 60 mg dry powder of MUS.

Step 2: 100 mg MUS was dissolved in 10 ml of 50 mM Calcium Acetate buffer, pH 6.7. An enzyme cocktail consisting of 5 mg Heparinase II and 5 mg of Heparinase III was added to this mixture, and the solution was maintained at 37° C. for 4 hours. The precipitate formed at the end of 2 hours was removed by centrifugation at 4000 RPM for 15 minutes. The supernatant of digested MUS was desalted in a size exclusion chromatography column.

Step 3: 100 mg MUS digested by the method explained above was loaded on a Im long, 10 cm diameter P10 size exclusion column. 500 mM Ammonium Acetate buffer was used as the running buffer. The eluent was tracked by absorption at UV 232 nM. 3 ml peaks were collected after the initial void volume. The peaks that gave absorption of more than 0.1 unit were collected. They were divided into 10 equal fractions. The different fractions were then lyophilized from water to get rid of ammonium bicarbonate salt. They were then assayed for the building blocks and functional characteristics (anti-Xa, and anti-IIa activity) by the assays described. Characteristics of Fraction 2 and Fraction 4 (named as M115, and M411) are listed below.

Results of Examples 1 and 2:

The methods described above were used to prepare and characterize the following LMWH compositions:

TABLE 1
LMWH compositions, AUC as determined by CE analysis.

| AUC % | M108 | M405 | M115 | M411 |
|---|---|---|---|---|
| 1 | 60.9 | 61.9 | 53.8 | 54.0 |
| 2 | 6.8 | 8 | 5.7 | 6.6 |
| 3 | 14.7 | 10.4 | 18.5 | 18.7 |
| 4 | 2.7 | 1.6 | 3.4 | 3.5 |
| 5 | 1.6 | 4.3 | 0.4 | 0.5 |
| 6 | 2.3 | 3.9 | 1.4 | 1.6 |
| 7 | 4.4 | 5.8 | 9 | 8.9 |
| 8 | 6.1 | 2.6 | 8.1 | 6.2 |
| 9 | 0.3 | 0.7 | | |
| 10 | 0.2 | 0.6 | | |
| Anti-Xa, IU/mg | 150 | 80 | 250 | 200 |
| Anti-IIa IU/mg | 130 | 0 | 200 | 130 |
| MW, Da | 5000 | 2200 | 5000 | 4500 |

TABLE 2
LMWH compositions, mole % of given components.

| Mole % | M108 | M405 | M115 | M411 |
|---|---|---|---|---|
| 1 | 62.7 | 67.0 | 55.3 | 56.7 |
| 2 | 6.3 | 7.7 | 5.2 | 6.2 |
| 3 | 12.6 | 9.3 | 15.8 | 16.3 |
| 4 | 2.3 | 1.4 | 2.9 | 3.0 |
| 5 | 1.0 | 2.8 | 0.2 | 0.3 |
| 6 | 1.0 | 1.7 | 0.6 | 0.7 |
| 7 | 2.6 | 3.6 | 5.3 | 5.3 |
| 8 | 11.1 | 5.0 | 14.7 | 11.5 |
| 9 | 0.3 | 0.8 | 250 | 200 |
| 10 | 0.2 | 0.6 | | |
| Anti-Xa, IU/mg | 150 | 80 | 250 | 200 |
| Anti-IIa IU/mg | 130 | 0 | 200 | 130 |
| MW. Da | 5,000 | 2200 | 5000 | 4500 |

A “grid” procedure was used for making M108, M405, M115, and M411, the specific examples mentioned above. It is to be understood that these are complex molecules obtained from a complex starting material by varying multiple parameters. Since the composition of the product is affected by multiple parameters, adjusting different parameters in different ways, and monitoring the profile of the product, would allow one of ordinary skill in the art to prepare products similar to M108, M405, M115, and M411.

The parameters that can be varied include, but are not limited to:

1) Starting material: UFH, FH, other LMWH preparations such as enoxaparin, dalteparin, certoparin parnaparin, nadroparin, reviparin, or tinzaparin, among others.

2) Salt (type, concentration): such as divalent metals such as Mg, and Ca (e.g., MgCl₂, Calcium acetate, etc.).

3) Enzyme (Heparinase I, II, III, mutant heparinases, and different combinations of these enzymes).

4) Temperature

5) Incubation time

This method has been used to create LMWH preparations with different characteristics. For instance, LMWH preparations which are neutralized by protamine can be created. For example, LMWH preparations M118 and M312 are both more sensitive to protamine neutralization of anti-factor Xa and anti-factor Ia activity than either UFH or enoxaparin. In addition, LMWH preparations with lower PF4 binding activity have been created, these preparations have lower amounts of components 1, 2, 4, and 6, which are associated with PF4 binding. Since PF4 binding has been linked to heparin induced thrombocytopenia (HIT), a composition of LMWH with decreased PF4 binding would be desirable.

PF4 binding was assayed using the filter binding assay of Maccarana et al. Briefly, 1 μg of 3H-radiolabeled heparin is incubated with 1 μg of PF4 in the presence of various amounts of nonradioactive LMWHs for 10 min at 37° C. in 10 μl of Tris buffer (130 mM NaCl, 50 mM Tris-HCl, pH 7.3). The volume is then made up to 300 μl by the addition of Tris buffer, and the samples are drawn through buffer-equilibrated cellulose nitrate filters on a vacuum manifold. The filters are washed with 2×5 ml of 130 mM NaCl, 50 mM Tris-HCl, and bound material eluted with 2×5 ml of 2 M NaCl, 50 mM Tris-HCl. On average greater than 99% of the radiolabeled material was removed from the filters with 2 M NaCl, 50 mM Tris-HCl.

To assess PF4 binding affinity for the various LMWHs, Scatchard analysis of the data collected by the filter binding assay was used. The lines of best fit and graphical equations for the data were determined. The gradients of these lines are equivalent to 1/Kd (1) and 1/Kd (2), the x intercept for the first line represents the number of binding sites on the protein (n1), and the x intercept for the second represents n1+n2, where n2 is the number of binding sites with Kd (2).

TABLE 3
Comparison of equivalent Anti-Xa activity for side effects

| Saccharide | | | |
|---|---|---|---|
| Components | enoxaparin (mg) | M118 (mg) | M312 (mg) |
| Total | 100 | 32.0 | 48.4 |
| 1 | 63.5 | 18.9 | 29.7 |
| 2 | 7.2 | 1.8 | 3.3 |
| 4 | 2.1 | 0.4 | 0.9 |
| 6 | 2.0 | 0.1 | 0.3 |
| Anti-Xa (IU/mg) | 100 | 100 | 100 |
| MW (Da) | 4,200 | 5,000 | 4,500 |

As is apparent from these results, the methods can be used to create a LMWH preparation with almost any characteristic desired, including varying ratios and levels of anti-factor Xa and anti-factor IIa activity; protamine neutralization; FGF binding; and PF4 binding.

### Example 3

### Analysis of LMWH in the Gastrointestinal Tract

The activity of enoxaparin at various points after delivery into the stomach of male Sprague Dawley rats was determined.

The rats (250 mg to 300 mg) were fed a standard rat chow and water and libium. The animals were fasted for 12 hours before the experiment. All animals were anesthetized with an intraperitoneal injection of ketamine (72 mg/kg) and acepromazine (3 mg/kg). Oral gavage was performed with a Rusch catheter attached to a 1-ml syringe. With the rat in an upright position, the dosing catheter was passed down the esophagus 10 cm from the incisors, and the dosing solution was expressed slowly into the stomach. Blood was drawn at different timepoints from 0-4 hours from a catheter inserted into the internal jugular vein. The animal was sacrificed at the end of the experiment.

10 mg of enoxaparin was incubated in rat gastric fluid for a period lasting from 15 minutes to 5 hours. An aliquot was withdrawn later to assay for its Xa/IIa activity. As seen in table 4, enoxaparin is stable in the acidic environment of stomach.

TABLE 4
Levels of anti-Xa and anti-IIa activity of enoxaparin

| | 0 Hours | 1 Hour | 2 Hours | 4 Hours |
|---|---|---|---|---|
| Anti-Xa | 100 | 100 | 100 | 100 |
| Anti-IIa | 25 | 25 | 25 | 25 |

LMWH with different overall charge to mass ratio have been generated. Shown in example 4 are the effect of charge to mass ratio on the rate of absorption of LMWH from the gastrointestinal tract. It has been found that charge to mass ratio is an important parameter that affects the oral delivery of heparin.

### Example 4

### Absorption of LMWHs in the Gastrointestinal Tract

LMWH was charge and size fractionated using a SAX column and Size exclusion column. 100 mg of LMWH was loaded on a 1 m tall, 10 cm diameter SAX column that holds 100 g of SAX resin. The compounds were eluted with 1 M NaCl buffer under gravimetric flow. 10 ml fractions of the eluent was collected from T=0 min. Eluting fractions were monitored for their LMWH content either by azure A assay or UV absorption at 232 nm wavelength. Peaks that showed a net absorption from 1 absorption unit to 100 absorption units were pooled together in 5 batches (Batch 1 through 5). These fractionated samples were then desalted and concentrated. The composition of the different fractions was measured by CE as well as by functional assays (Xa, IIa). The charge to mass ratio was calculated as follows. Assuming a charge of 2.3 per disaccharide molecule, the charge/Mass ratio is the charge of the molecule divided by the mass of the molecule.

Thus charge to mass of enoxaparin=19.32/4200

Charge to mass for M108=23/5000

Charge to mass for M405=10.12/2200

Enoxaparin, M108 or M405 was then introduced into the gastrointestinal tract by intraduodenl delivery and absorption of these LMWH was determined. Briefly, male Sprague Dawley rats (250 mg to 300 mg) were fed a standard rat chow and water ad libium. The animals were fasted for 12 h before the experiment. All animals were anesthetized with an intraperitoneal injection of ketamine (72 mg/kg) and acepromazine (3 mg/kg). Delivery was performed with a Rusch catheter attached to a 1-ml syringe. The dosing catheter inserted into the duodenum, and the dosing solution was expressed slowly into the duodenum. Blood was drawn at different timepoints from 0-4 hours. The animal was sacrificed at the end of the experiment.

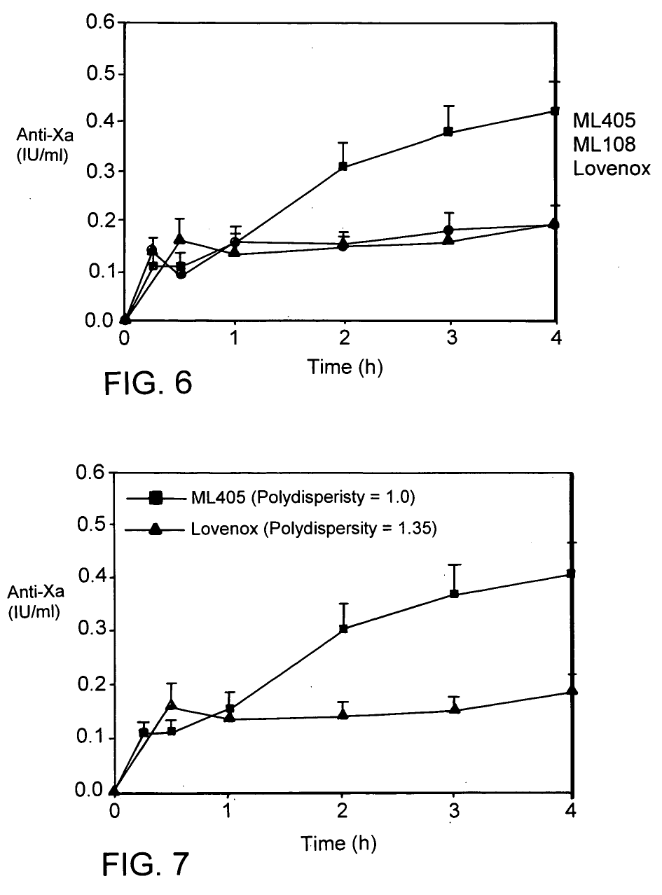

As seen in FIG. 6, there is a correlation between the charge to mass ratio and the absorption rate of LMWH from GIT.

### Example 5

### Effect of Polydispersity on the pK Profile of LMWH

LMWH was fractionated as a function of its polydispersity using a Size exclusion column. 100 mg of LMWH was loaded on a 1 m tall, 10 cm diameter size exclusion column that holds 100 g of P10 size exclusion resin. The compounds were eluted with ammonium bicarbonate buffer under gravimetric flow. 10 ml fractions of the eluant was collected from T=0 minutes. Eluting fractions were monitored for their LMWH content by UV absorption at 232 nm wavelength. Samples that showed positive absorption >0.1 Unit were pooled together as 4 different batches. These fractionated samples were then desalted and concentrated. Polydispersity of the samples were measured by Multiangle laser light scattering. M108 has a polydisperisty of 1.0 while enoxaparin has a polydisperisty of 1.3.

Enoxaparin or M108 was then introduced into the gastrointestinal tract by intraduodenl delivery and absorption of these LMWH was determined. Briefly, male Sprague Dawley rats (250 mg to 300 mg) were fed a standard rat chow and water ad libium. The animals were fasted for 12 h before the experiment. All animals were anesthetized with an intraperitoneal injection of ketamine (72 mg/kg) and acepromazine (3 mg/kg). Delivery was performed with a Rusch catheter attached to a 1 ml syringe. The dosing catheter inserted into the duodenum, and the dosing solution was expressed slowly into the duodenum. Blood was drawn at different timepoints from 0-4 hours. The animal was sacrificed at the end of the experiment.

As seen in FIG. 7, there is a correlation between the polydispersity and the absorption rate of LMWH from GIT.

### Example 6

### Pulmonary Delivery of Enoxaparin

The pulmonary delivery of enoxaparin to rabbits was investigated and the derived pharmacokinetic parameters compared to those for a standard subcutaneous injection. A grid search of different chemical and physical parameters was investigated to identify favorable conditions for pulmonary delivery of enoxaparin. The grid search conditions were determined based on an analysis of the physical and chemical properties such as polydispersity, charge to mass ratio and sulfation patterns of enoxaparin. To examine the effect of multiple blood withdrawals and pulmonary inhalation on the hematology of circulation, blood samples were collected in the beginning and at the end of the experiments. For examination of the potential pathological effects of inhaled formulation on the lung histology, the lungs of rabbits were harvested at the end of the experiments and examined using standard histochemical techniques.

Animals:

For the rabbit model, 2.5-3 kg New Zealand male rabbits were used with 3 rabbits per group. Rabbits were allowed to adapt for 7 days and free access to water and food. Ketamine (40 mg/kg) and Xylazine (5 mg/kg) were used to anesthetize the rabbits. To withdraw blood samples, a 24-gauze Teflon catheter was inserted into the center auricular artery. The catheter was connected to a heparin cap filled with 0.9% saline solution.

Physical Formulation of Enoxaparin for Pulmonary Delivery:

Chemically formulated particles of enoxaparin were prepared using a commercially available enoxaparin preparation and a salt concentration of 3-30 mM, followed by lyophilization and grinding by using a mortar and pestal or other physical processes to granulate the solid powder. This powder was then subjected to size separation by sieving through mesh sizes of 20, 53, 75 and 106 μm. Powders with sizes ranging from 20-53 μm, 53-75 μm and 53-106 μm were collected and used for pulmonary delivery.

Measurement of Particle Size.

Particle size was measured using a Coulter LS230 laser diffraction instrument, manufactured by Beckman Coulter (Miami, Fla. 33116). Particle size can be measured either in the “wet” or in the “dry” mode with about the same amount of accuracy. The LMWH samples were primarily run in the “wet” mode, with kerosene being the suspension medium. Pump flow was maintained at 50%. The particles flowing through the orifice diffract the laser beam; the extent of diffraction is a function of particle size.

Pulmonary Delivery of Enoxaparin:

A 15-cm tracheal tube was inserted into the trachea of the anesthetized rabbits via mouth. Subsequently, the insufflator attached to a straight delivery tube of equal length to that of tracheal tube was inserted through the tracheal tube. LMWH was delivered at doses of 3 and 6 mg/kg, the amount of powder was derived by subtracting the weight of insufflator before and after delivery. 0.2 ml of blood was withdrawn 0, 5, 10, 30 minutes, 1, 2, 3, 4, 6, 8, 10, 12, 14, 18, 24 hours after the inhalation. The first 0.2 ml blood withdrawn was discarded with each withdraw. Blood samples were collected in an aqueous solution of sodium citrate (3.8%; 1/9, v/v), centrifuged at 2000×g for 20 min and the resulting plasma was shock frozen and stored in −80° C. freezer until anti-Xa assays could be completed.

Subcutaneous administration of Enoxaparin:

Enoxaparin at doses of 3 and 6 mg/kg was given by subcutaneous injection at time 0. Blood samples were collected 0, 3, 5, 10, 15, 30 min, 1, 2, 3, 4, 6, 8, 12 hours after subcutaneous injection and processed as described above. The plasma was collected at indicated times and analyzed for anti-Xa assay as described. Enoxaparin for subcutaneous injection was formulated as a solution in PBS or water at 100 mg/ml concentration and appropriate doses were injected.

Anti-Xa Activity Assays:

An anti-Xa assay was used to monitor plasma LMWH levels. The anti-Xa assay was performed using a modification of the amidolytic method of Teien and Lie (Thrombosis res. 10: 399-410, 1977) with the Coatest heparin test kit by using S-2222 as the chromogenic substrate (Diapharma Group, Inc. OH). The detailed procedure is described elsewhere (Liu, etc., PNAS, 94: 1739-1744, 1997). The concentration of enoxaparin in unknown samples was calculated by comparing readings to the calibration curve which was linear in the range of 0.1-0.7 IU/ml.

Calculation of Pharmacokinetic Parameters:

Experimental data, expressed in anti-Xa IU/mL, was utilized for non-linear regression curves based on a one-compartment model (Comelli and Fareed, Semin thromb Hemost, 25: 57-61, 1999) by using SigmaPlot program with the method of extended least squares. From the kinetic curves, the following parameters were calculated: the area under curve (AUC expressed in IU*hr*ml⁻¹), absorption half-life (t_{1/2} expressed in hr); and half-life of apparent elimination (t_{1/2}e expressed in hr). The AUC (0-t) was calculated using the trapezoidal rule (Rowland and Tozer, Clinical Pharmacokinetics. Concepts and Applications. 459-461, Lea and Febiger, 1989) and extrapolated to infinity (AUC) by dividing the value of the last measured concentration by the elimination rate constant.

TABLE 4
Physical properties ofEnoxaparin particles:

| | Geometric Diameter | | |
|---|---|---|---|
| Fraction | (μm) | Mass Density (g/cm³) | Porosity |
| 20-53 | 42 | 0.4 ± 0.1 | Nonporous |
| 53-75 | 80 | 0.4 ± 0.1 | Nonporous |
| 75-106 | 102 | 0.4 ± 0.1 | Nonporous |

To study if enoxaparin could be delivered through a pulmonary route, chemically formulated enoxaparin was generated and sieved the generated particles (1-500 μm) with sieves of different mesh sizes (20, 53, and 75 μm cut-offs). Enoxaparin particles of diameter ranges from 20-53, 53-75, and 75-106 μm were obtained. These particles were administered by inhalation to rabbits with an insufflator at 3 and 6 mg/kg via an intubated trachea tube. The same doses were also injected subcutaneously for reference. Counter to current thinking particles delivered through the intrapulmonary route showed significant, fast absorption as indicated by anti-Xa activity assay of the plasma samples (Table 5). The characteristics shared by these particles include—(1) an extremely short absorption half-life (1-10 min), compared to 1-2 hours for subcutaneous administration (tₘₐₓ was reached at about 30 minutes after inhalation); (2) comparable or slower elimination rates to subcutaneous administration (the elimination half-lives of the tested particles range from about 2-6 hours, which is generally slower than that of subcutaneous administration); (3) significant bioavailability (the relative bioavailability to subcutaneous administration ranges from about 30-60%), and (4) linear dose-response relationships (Table 5). Finally, the mean residence time (MRT) value of enoxaparin particles was approximately equivalent to subcutaneous administration (Table 5).

TABLE 5
Pharmacokinetic parameters for subcutaneous and pulmonary
delivery of Enoxaparin

| | | | Pulmonary | |
|---|---|---|---|---|
| | s.c. injection | | (d_{avg} = 42 μm) | |
| | 3 mg/kg | 6 mg/kg | 3 mg/kg | 6 mg/kg |
| Kₐ | 0.40 | 0.35 | 8.53 | 8.37 |
| Kₑ | 0.35 | 0.30 | 0.22 | 0.11 |
| AUC | 6.27 | 13.45 | 2.20 | 5.81 |
| (IU * hr/mL) | | | | |
| MRT (AUMC/AUC) | 4.75 | 5.48 | 3.77 | 6.18 |

### Example 7

### Chemical Composition of Enoxaparin Affects pK Profile

The pulmonary delivery to rabbits of two different chemical compositions of LMWH was investigated. Except for the chemical formulation of enoxaparin for intrapulmonary delivery (including such parameters as pH, addition and stoichiometric quantity of counterions) the creation of particles in the range of 20-53 μm for intrapulmonary delivery was identical. Formulation 2 had a lower counter ion concentration (about 50% lower) and is formulated at a slightly higher pH. Thus, identical drying, grinding, and sieving procedures to derive particles were observed. Each was delivered via an intrapulmonary route to New Zealand rabbits at a dose of 3 mg/kg. The two formulations differ in a number of ways: (1) the pH of formulation #2 is higher than that of formulation #1 and the counter ion concentration is lower by one-half to one-third. More unmasked negative charge leads to a “burst” effect enabling faster absorption characteristics (Table 6) Similarly changes in the nature of the counterion “neutralization” agent affect the pharmacokinetic profile.

Different chemical formulations of enoxaparin with identical physical formulation parameters have markedly different pharmacokinetic parameters (FIG. 6 and Table 6). Changing the chemical formulation of enoxaparin can markedly affect parameters important in the clinical use of enoxaparin including bioavailability, Cmax and elimination rates.

TABLE 6
Pharmacokinetic parameters intrapulmonary delivery of
enoxaparin using two different chemical formulations

3 mg/kg pulmonary delivery

| | Formulation #1 | Formulation #2 |
|---|---|---|
| Kₐ | 8.53 | 7.60 |
| Kₑ | 0.22 | 0.22 |
| AUC (IU * hr/mL) | 2.20 | 4.80 |
| MRT (AUMC/AUC) | 3.8 | 4.1 |

### Example 8

### Pulmonary Delivery of Dalteparin

Pulmonary delivery for a second commercially available LMWH, dalteparin, was also investigated. Chemically formulated dalteparin was delivered intrapulmonary to rabbits at doses of 3 and 6 mg/kg and the derived pharmacokinetic parameters compared to those for a standard subcutaneous injection. Similar to studies completed on enoxaparin, a grid search of different chemical and physical parameters was investigated to identify favorable conditions for pulmonary delivery of dalteparin. The grid search conditions were determined based on an analysis of the physical and chemical properties such as polydispersity, charge to size ratio and sulfation patterns of dalteparin. In this case, not only was the concentration of counterion and pH varied, but also the nature of the masking agent. In this case, a grid search of possible masking agents indicated that harder Lewis acids, specifically transition metals and/or divalent metal ions resulted in longer lasting formulations with generally lower bioavailability.

Animals:

For the rabbit model, 2.5-3 kg New Zealand male rabbits were used with 3 rabbits per group. Prior to pulmonary or subcutaneous delivery rabbits were treated in an identical fashion to rabbits given doses of enoxaparin (see above).

Physical Formulation of Dalteparin for Pulmonary Delivery:

Chemically formulated particles of dalteparin were prepared using a commercially available dalteparin preparation and a salt concentration of 3-30 mM, followed by lyophilization and grinding to granulate the solid powder. This powder was then subjected to size separation by sieving through mesh sizes of 20, 53, 75 and 106 μm. Powders with sizes ranging from 20-53 μm, 53-75 μm and 75-106 μm were collected and used for pulmonary delivery.

Other aspects of the experimental procedure are identical to example 6.

To study if dalteparin could be delivered through a pulmonary route, chemically formulated dalteparin was ground and sieved the generated particles (1-500 μm) with sieves of different mesh sizes (20, 53, 75 and 106 μm cut-offs). Dalteparin particles of diameter ranges from 20-53, 53-75 μm and 75-106 μm were obtained. These particles were administered by inhalation to rabbits with an insufflator at 3 and 6 mg/kg via an intubated trachea tube. The same doses were also injected subcutaneously for reference. Once again, counter to current thinking, both particles tested showed significant, fast absorption as indicated by anti-Xa activity assay of the plasma samples (Table 7). The characteristics shared by these formulated dalteparin particles include—(1) an extremely short absorption half-life (1-10 min), compared to 1-2 hours for subcutaneous administration (tₘₐₓ was reached at about 30 minutes after inhalation); (2) comparable or slower elimination rates to subcutaneous administration (the elimination half-lives of the tested particles range from about 2-6 hours, which is generally slower than that of subcutaneous administration); (3) significant bioavailability (the relative bioavailability to subcutaneous administration ranges from about 30-60%), and (4) linear dose-response relationships (the higher dose is associated with higher peak concentration (Cₘₐₓ) (Table 7). Finally, the mean residence time (MRT) value of formulated dalteparin particles was approximately equivalent to s.c. administration (Table 7).

TABLE 7
Pharmacokinetic parameters for subcutaneous and
pulmonary delivery of dalteparin

| | s.c. | | pulmonary | | pulmonary | |
|---|---|---|---|---|---|---|
| | injection | | divalent | | transition metal | |
| | 3 mg/kg | 6 mg/kg | 3 mg/kg | 6 mg/kg | 3 mg/kg | 6 mg/kg |
| Kₐ | 0.40 | 0.32 | 6.71 | 6.42 | 3.09 | 6.22 |
| Kₑ | 0.33 | 0.27 | 0.20 | 0.16 | 0.26 | 0.15 |
| AUC | 6.93 | 13.27 | 2.28 | 5.82 | 2.08 | 2.99 |
| (IU*hr/mL) | | | | | | |
| MRT | 4.97 | 5.93 | 3.43 | 5.71 | 3.27 | 6.06 |
| (AUMC/AUC) | | | | | | |

### Example 9

### Chemical Composition of Dalteparin Affects pK Profile

The pulmonary delivery to rabbits of two different chemical compositions of dalteparin was investigated. Except for chemical formulation of dalteparin for pulmonary delivery (including such parameters as pH, addition and stoichiometric quantity of counterions) the creation of particles in the range of 20-53 μm for pulmonary delivery was identical. Formulation 2 possessed a higher pH and generally a lower amount of counter ion. Thus, identical drying, grinding, and sieving procedures to derive particles were observed. Each was delivered via a pulmonary route to New Zealand rabbits at a dose of 3 mg/kg.

As was the case with enoxaparin, different chemical formulations of dalteparin with identical physical formulation parameters have markedly different pharmacokinetic parameters (FIG. 7 and Table 8). Changing the chemical formulation of dalteparin can markedly affect parameters, including bioavailability, Cₘₐₓ and elimination rates, important in the clinical use of dalteparin given through the pulmonary route.

TABLE 8
Pharmacokinetic parameters pulmonary delivery of dalteparin
using two different chemical formulations

3 mg/kg pulmonary delivery

| | Formulation #1 | Formulation #2 |
|---|---|---|
| Kₐ | 6.7 | 2.6 |
| Kₑ | 0.20 | 0.36 |
| AUC (IU * hr/mL) | 2.3 | 3.5 |
| MRT (AUMC/AUC) | 3.43 | 3.0 |

### Example 10

### Pulmonary Delivery of Other LMWHs

Pulmonary delivery of other low molecular weight heparins, namely M118 and M312, were also investigated. Chemically formulated particles of M118 and M312 were prepared using a salt concentration of 10-30 mM, followed by lyophilization and grinding by using a coffee grinder to granulate the solid powder Chemically formulated M118 and M312 was delivered intrapulmonary to rabbits at doses of 3 mg/kg and the pharmacokinetic parameters were derived and compared to subcutaneous injection at the same dose. Similar to studies completed on dalteparin and enoxaparin, a grid search of different chemical parameters was investigated to identify favorable conditions for pulmonary delivery of M118 and M312. The grid search conditions were determined based on an analysis of the physical and chemical properties such as polydispersity, charge to size ratio and sulfation patterns of M118 and M312.

Animals:

For the rabbit model, 2.5-3 kg New Zealand male rabbits were used with 3 rabbits per group.

Physical Formulation of M118 and M312 for Pulmonary Delivery:

Chemically formulated particles of M1181 and M312 were prepared by using a coffee grinder to granulate lyophilized solid powder. This powder was then subjected to size separation by sieving through mesh sizes of 20, 53, 75 and 106 μm. Powders were collected and used for pulmonary delivery.

Other aspects of the experimental procedure are identical to example 6.

To study if M118 and M312 could be delivered through a pulmonary route, chemically formulated M118 and M312 were ground and sieved the generated particles (1-500 μm) with sieves of different mesh sizes (20, 53, 75 and 106 μm cut-offs). M118 and M312 particles of diameter ranges were obtained. These particles were administered by inhalation to rabbits with an insufflator at 3 and 6 mg/kg via an intubated trachea tube. The same doses were also injected subcutaneously for reference. Once again, counter to current thinking, both particles tested showed significant, fast absorption as indicated by anti-Xa activity assay of the plasma samples (Table 10). Thus, the characteristics shared by all low molecular weight heparin particles include—(1) an extremely short absorption half-life (1-10 min), compared to 1-2 hours for subcutaneous administration (tₘₐₓ was reached at about 30 minutes after inhalation); (2) comparable or slower elimination rates to subcutaneous administration (the elimination half-lives of the tested particles range from about 2-6 antihours, which is generally slower than that of subcutaneous administration); (3) and a mean residence time (MRT) value that is approximately equivalent to subcutaneous administration (Table 9).

TABLE 9
Pharmacokinetic parameters for subcutaneous and pulmonary delivery
of M118 and M312

| | 3 mg/kg | | | |
|---|---|---|---|---|
| | | | M118 | M312 |
| | M118 s.c. | M312 s.c. | pulmonary | pulmonary |
| Kₐ | 0.34 | 0.47 | 7.5 | 5.9 |
| Kₑ | 0.29 | 0.41 | 0.34 | 0.34 |
| AUC | 14.8 | 9.2 | 5.0 | 3.8 |
| (IU * hr/mL) | | | | |
| MRT | 5.4 | 3.9 | 2.4 | 2.6 |
| (AUMC/AUC) | | | | |

All references, patents and patent publications that are recited in this application are incorporated in their entirety herein by reference.

What is claimed is:

1. A method for preparing a polysaccharide for non-invasive delivery, comprising:
neutralizing a polysaccharide, to thereby prepare the polysaccharide for non-invasive delivery.

2. The method of claim 1, further comprising reducing the mass of the polysaccharide.

3. The method of claim 1, further comprising determining a chemical signature for the polysaccharide, and neutralizing the polysaccharide based upon its chemical signature.

4. The method of claim 3, further comprising reducing the mass of the polysaccharide based upon its chemical signature.

5. The method of claim 1, wherein the net negative or net positive charge of the polysaccharide is reduced by at least 30% or more.

6. The method of claim 1, wherein the polysaccharide is neutralized such that there is a net negative and net positive charge of 0.

7. The method of claim 3, wherein the polysaccharide is neutralized by digesting the polypeptide with at least one agent which cleaves the polysaccharide at known locations in the polysaccharide based upon it chemical signature.

8. The method of claim 7, wherein the agent is an enzyme.

9. The method of claim 8, wherein the enzyme is a heparin lyase.

10. The method of claim 9, wherein the heparin lyase is selected from the group consisting of heparinase I, heparinase II, heparinase III, heparinase IV, heparanase, and functionally active fragments and variants thereof.

11. The method of claim 7, wherein the agent is a chemical.

12. The method of claim 11, wherein the chemical is selected from group consisting of oxidative depolymerization with H₂O₂ or Cu⁺ and H₂O₂, deaminative cleavage with isoamyl nitrite, or nitrous acid, and 1-eliminative cleavage with benzyl ester of heparin by alkaline treatment.

13. The method of claim 1, wherein the polysaccharide is neutralized by contacting the polysaccharide with a charge neutralizing agent.

14. The method of claim 13, wherein the charge neutralizing agent is a counter ion.

15. The method of claim 14, wherein the counterion is selected from the group consisting of barium, calcium, sodium, potassium, lithium, ammonium, magnesium, and zinc.

16. The method of claim 13, wherein the charge neutralizing agent is a transition metal.

17. The method of claim 16, wherein the transition metal is selected from the group consisting of iron, nickel and copper.

18. The method of claim 13, wherein the charge neutralizing agent is selected from the group consisting of a small organic compound, spermine, spermidine, low molecular weight protamine, and a basic peptide.

19. The method of claim 1, wherein the polysaccharide is a HLGAG.

20. The method of claim 1, further comprising creating particles of the polysaccharide.

21. The method of claim 20, wherein the particles have a mean geometric diameter of about 1-500 microns.

22. The method of claim 1, further comprising formulating the polysaccharide with a delivery enhancer.

23. The method of claim 22, wherein the delivery enhancer is selected from the group consisting of a surfactant, an absorption enhancer, and a polymer.

24. A method for preparing a heparin, for non-invasive in vivo delivery, comprising:
neutralizing a heparin, to thereby prepare the heparin for non-invasive in vivo delivery.

25. The method of claim 24, further comprising reducing mass of the heparin.

26. The method of claim 24, wherein the heparin to be neutralized is unfractionated heparin, fractioned heparin or a synthetic pentasaccharide.

27. The method of claim 26, wherein the fractionated heparin is LMWH.

28. The method of claim 27 wherein the LMWH is selected from the group consisting of enoxaparin, dalteparin, reviparin, tinzaparin, nadroparin, certoparin, ardeparin, and parnaparin.

29. The method of claim 26, further comprising determining a chemical signature for heparin and neutralizing the heparin based upon its chemical signature.

30. The method of claim 29, further comprising reducing the mass of the heparin based upon its chemical signature.

31. The method of claim 29, wherein the net negative charge of the heparin is reduced by at least 30% or more.

32. The method of claim 29, wherein the heparin is neutralized such that there is a total net charge of 0.

33. The method of claim 29, wherein the heparin is neutralized by digesting the heparin with at least one agent selected based upon the chemical signature of the heparin.

34. The method of claim 33, wherein the heparin is neutralized such that at least one biological activity of the heparin is maintained or enhanced.

35. The method of claim 34, wherein the activity is anti-Xa activity, anti-IIa activity, protamine neutralization, FGF-2 binding activity, platelet factor 4 binding activity or other measures of HIT propensity, or combinations thereof.

36. The method of claim 33 or 34, wherein the agent is an enzyme.

37. The methods of claim 36, wherein the enzyme is a heparin lyase.

38. The method of claim 37, wherein the heparin lyase is selected from the group consisting of heparinase I, heparinase II, heparinase III, heparinase IV, heparanase, and functionally active fragments and variants thereof.

39. The method of claim 33 or 34, wherein the agent is a chemical.

40. The method of claim 39, wherein the chemical is selected from the group consisting of oxidative depolymerization with H₂O₂ or Cu⁺ and H₂O₂, deaminative cleavage with isoamyl nitrite, or nitrous acid, and β-eliminative cleavage with benzyl ester of heparin by alkaline treatment.

41. The method of claim 29, wherein the heparin is neutralized by contacting the heparin with a charge neutralizing agent.

42. The method of claim 41, wherein the charge neutralizing agent is a counter ion such as mono- or divalent ion.

43. The method of claim 42, wherein the counterion is selected from the group consisting of barium, calcium, sodium, potassium, lithium, ammonium, and magnesium.

44. The method of claim 41, wherein the charge neutralizing agent is a transition metal.

45. The method of claim 44, wherein the transition metal is selected from the group consisting of iron, nickel, zinc and copper.

46. The method of claim 41, wherein the charge neutralizing agent is a small organic compound, spermine, spermidine, low molecular weight protamine, or a basic peptide.

47. The method of claim 30, wherein the heparin is derived from enoxaparin and the enoxaparin is neutralized and the mass reduced such that the heparin has a charge to mass ratio that is less than 19.32/4200.

48. The method of claim 30, wherein the heparin is derived from nadroparin and the nadroparin is neutralized and the mass reduced such that the heparin has a charge to mass ratio that is less than 27.6/6000.

49. The method of claim 30, wherein the heparin is derived from dalteparin and the dalteparin is neutralized and the mass reduced such that the heparin has a charge to mass ratio that is less than 23/6000.

50. The method of claim 30, wherein the heparin is derived from reviparin and the reviparin is neutralized and the mass reduced such that the heparin has a charge to mass ratio that is less than 25.3/5500.

51. The method of claim 30, wherein the heparin is derived from parnaparin and the parnaparin is neutralized and the mass reduced such that the heparin has a charge to mass ratio that is less than 30.4/6610.

52. The method of claim 30, wherein the heparin is derived from tinzaparin and the tinzaparin is neutralized and the mass reduced such that the heparin has a charge to mass ratio that is less than 28.06/6100.

53. The method of claim 30, wherein the heparin is derived from enoxaparin and the net charge of the heparin is less than about 18.

54. The method of claim 30, wherein the heparin is derived from dalteparin and the net charge of the heparin is less than about 21.

55. The method of claim 30, wherein the heparin is derived from nadroparin and the net charge of the heparin is less than about 26.

56. The method of claim 30, wherein the heparin is derived from reviparin and the net charge of the heparin is less than about 24.

57. The method of claim 30, wherein the heparin is derived from parnaparin and the net charge of the heparin is less than about 29.

58. The method of claim 30, wherein the heparin is derived from tinzaparin and the net charge of the heparin is less than about 27.

59. The method of claim 24, wherein the heparin is prepared for a non-invasive delivery selected from the group consisting of: pulmonary delivery, transdermal delivery and mucosal delivery.

60. A method for preparing a polysaccharide for non-invasive in vivo delivery, comprising:
providing a polysaccharide;
determining a chemical signature for the polysaccharide; and
reducing the mass of the polysaccharide based upon its chemical signature, to thereby prepare the polysaccharide for non-invasive in vivo delivery.

61. The method of claim 60, wherein the mass of the polysaccharide is reduced such that at least one or more activity of the polysaccharide is at least partially maintained.

62. The method of claim 60, wherein the mass of the polysaccharide is reduced such that at least one activity of the polysaccharide is at least partially maintained and at least one other activity of the polysaccharide is reduced.

63. The method of claim 60, further comprising neutralizing the polysaccharide.

64. The method of claim 60, wherein the mass of the polysaccharide is reduced at least 20% from the mass of the provided polysaccharide.

65. The method of claim 60, wherein the mass of the polysaccharide is reduced by at least 100 Da or more from the mass of the provided polysaccharide.

66. The method of claim 63, wherein the net negative or net positive charge of the polysaccharide is reduced by at least 30% or more.

67. The method of claim 63, wherein the polysaccharide is neutralized such that there is a net negative and net positive charge of 0.

68. The method of claim 60, wherein the mass of the provided polysaccharide is reduced by digesting the polypeptide with at least one agent selected based upon the chemical signature of the polysaccharide.

69. The method of claim 68, wherein the agent is an enzyme.

70. The method of claim 69, wherein the heparin is a heparin lyase.

71. The method of claim 70, wherein the heparin lyase is selected from the group consisting of heparinase I, heparinase II, heparinase III, heparinase IV, heparanase, and functionally active fragments and variants thereof.

72. The method of claim 68, wherein the agent is a chemical.

73. The method of claim 72, wherein the chemical is selected from group consisting of oxidative depolymerization with H₂O₂ or Cu⁺ and H₂O₂, deaminative cleavage with isoamyl nitrite, or nitrous acid, and β-eliminative cleavage with benzyl ester of heparin by alkaline treatment.

74. The method of claim 60, wherein the polysaccharide is a HLGAG.

75. A method for preparing a heparin for non-invasive in vivo delivery, comprising:
providing a heparin;
determining a chemical signature for the heparin; and
reducing the mass of the heparin based upon its chemical signature, to thereby prepare the heparin for in vivo mucosal delivery.

76. The method of claim 75, wherein the mass of the heparin, is reduced such that at least one or more activity of the heparin is at least partially maintained or enhanced.

77. The method of claim 76, wherein the activity is one or more of anti-Xa activity, anti-IIa activity, PF4 activity or other measure of HIT propensity, FGF-2 activity, protamine neutralization.

78. The method of claim 76, wherein the mass of the heparin is reduced such that at least one other activity of the heparin is reduced.

79. The method of claim 78, wherein PF4 binding or other measure of HIT propensity is reduced

80. The method of claim 75, further comprising neutralizing the heparin.

81. The method of claim 75, wherein the mass of the heparin, is reduced at least 10% from the mass of the provided heparin.

82. The method of claim 75, wherein the mass of the heparin is reduced by at least 100 Da or more from the mass of the provided heparin.

83. The method of claim 75 or 80, wherein the heparin is LMWH.

84. The method of claim 83, wherein the LMWH is derived enoxaparin and wherein the mass of enoxaparin is reduced such that the mass is about 4100 Da or less.

85. The method of claim 83, wherein the LMWH is derived from nadroparin and wherein the mass of nadroparin is reduced such that the mass is about 5900 Da or less.

86. The method of claim 83, wherein the LMWH is derived from dalteparin and wherein the mass of dalteparin is reduced such that the mass is about 4700 Da or less.

87. The method of claim 83, wherein the LMWH is derived from reviparin and wherein the mass of reviparin is reduced such that the mass is about 5400 Da or less.

88. The method of claim 83, wherein the LMWH is derived from parnaparin and wherein the mass of parnaparin is reduced such that the mass is about 6,500 Da or less.

89. The method of claim 83, wherein the LMWH is derived from tinzaparin and wherein the mass of tinzaparin is reduced such that the mass is about 6,000 Da or less.

90. The method of claim 80, wherein the net negative or net positive charge of the heparin is reduced by at least 10% or more.

91. The method of claim 80, wherein the heparin, is neutralized such that there is a net negative and net positive charge of 0.

92. The method of claim 75, wherein the mass of the provided heparin is reduced by digesting the heparin with at least one agent selected based upon the chemical signature of the heparin.

93. The method of claim 92, wherein the agent is an enzyme.

94. The method of claim 93, wherein the enzyme is a heparin lyase.

95. The method of claim 94, wherein the heparin lyase is selected from the group consisting of heparinase I, heparinase II, heparinase III, heparinase IV, heparanase, and functionally active fragments and variants thereof.

96. The method of claim 92, wherein the agent is a chemical.

97. The method of claim 96, wherein the chemical is selected from group consisting of oxidative depolymerization with H₂O₂ or Cu⁺ and H₂O₂, deaminative cleavage with isoamyl nitrite, or nitrous acid, and β-eliminative cleavage with benzyl ester of heparin by alkaline treatment.

98. The method of claim 83, wherein the LMWH is derived from enoxaparin or unfractionated heparin and the enoxaparin or unfractionated heparin is neutralized and the mass reduced such that the LMWH has a charge to mass ratio of less than 19.32/4200.

99. The method of claim 83, wherein the LMWH is derived from nadroparin or unfractionated heparin and the nadroparin or unfractionated heparin is neutralized and the mass reduced such that the LMWH has a charge to mass ratio of less than 27.6/6000.

100. The method of claim 83, wherein the LMWH is derived from dalteparin or unfractionated heparin and the dalteparin or unfractionated heparin is neutralized and the mass reduced such that the LMWH has a charge to mass ratio of less than 23/5000.

101. The method of claim 83, wherein the LMWH is derived from reviparin or unfractionated heparin and the reviparin or unfractionated heparin is neutralized and the mass reduced such that the LMWH has a charge to mass ratio of less than 25.3/5500.

102. The method of claim 83, wherein the LMWH is derived from parnaparin or unfractionated heparin and the parnaparin or unfractionated heparin is neutralized and the mass reduced such that the LMWH has a charge to mass ratio of less than 30.4/6610.

103. The method of claim 83, wherein the LMWH is derived from tinzaparin or unfractionated heparin and the tinzaparin or unfractionated heparin is neutralized and the mass reduced such that the LMWH has a charge to mass ratio of less than 28.06/6100.

104. The method of claim 75, wherein the heparin is prepared for non-invasive delivery selected from the group consisting of pulmonary delivery, transdermal delivery and mucosal delivery.

105. A polysaccharide composition for non-invasive in vivo delivery made by the method of claim 1 or 24.

106. A composition for oral delivery of a heparin wherein the heparin has a net negative charge which is less than a reference net charge for the heparin.

107. The composition of claim 106, wherein the heparin has a mass which is less than a reference mass for heparin.

108. The composition of claim 106 or 107, wherein the heparin is a LMWH.

109. The composition of claim 108, wherein the reference net charge is the net charge of enoxaparin.

110. The composition of claim 109, wherein the net charge of the LMWH is at least 30% less than the net charge of enoxaparin.

111. The composition of claim 110, wherein the net charge of the LMWH is less than 18.

112. The composition of claim 107, wherein the reference mass is the mass of enoxaparin and the mass of the LMWH is at least 30 Da less than the mass of enoxaparin.

113. The composition of claim 112, wherein the mass of the LMWH is about 4100 Da or less.

114. The composition of claim 106, wherein the heparin is in a dry particle formulation.

115. The method of claim 114, wherein the dry particle has a mean geometric diameter of at least 5 microns or more.

116. The composition of claim 106, wherein the heparin is in an aqueous formulation.

117. The composition of claim 106, further comprising a charge neutralization agent.

118. A composition for pulmonary delivery of a heparin, comprising a heparin and a charge neutralizing agent, wherein the heparin has a net negative charge which is less than a reference net charge for the heparin.

119. The composition of claim 118, wherein the heparin has a mass which is less than a reference mass for heparin.

120. The composition of claim 118 or 119, wherein the heparin is a LMWH.

121. The composition of claim 120, wherein the reference net charge is the net charge of enoxaparin.

122. The composition of claim 121, wherein the net charge of the LMWH is at least 30% less than the net charge of enoxaparin.

123. The composition of claim 121, wherein the net charge of the LMWH is less than 18.

124. The composition of claim 119, wherein the reference mass is the mass of enoxaparin and the mass of the LMWH is at least 30 Da less than the mass of enoxaparin.

125. The composition of claim 118, wherein the heparin is in a dry particle formulation.

126. The composition of claim 118, wherein the heparin is in an aqueous formulation.

127. The composition of claim 117 or claim 118, wherein the charge neutralization agent is a counter ion.

128. The composition of claim 127, wherein the counter ion is selected from the group consisting of barium, calcium, sodium, potassium, lithium, ammonium, magnesium, zinc).

129. The composition of claim 117 or claim 118, wherein the charge neutralizing agent is a transition metal.

130. The composition of claim 129, wherein the transition metal is selected from the group consisting of iron, nickel, and copper.

131. The composition of claim 117 or claim 118, wherein the charge neutralizing agent is selected from the group consisting of spermine, spermidine, low molecular weight protamine, and a basic peptide.

132. The composition of claim 106 or claim 118, wherein the composition further comprises an active agent.

133. The composition of claim 132, wherein the active agent and the heparin are distinct.

134. The composition of claim 106 or claim 118, wherein the heparin is a carrier and an active agent.

135. The composition of claim 106 or claim 118, further comprising a delivery enhancer.

136. A composition for mucosal delivery comprising M405.

137. A composition for mucosal delivery comprising M108.

138. A method for delivering a sulfonated polysaccharide to a subject, comprising:
orally administering to a subject a sulfonated polysaccharide in a therapeutically effective amount, to thereby deliver to polysaccharide to the subject.

139. The method of claim 138, wherein the sulfonated polysaccharide is heparin.

140. The method of claim 139, wherein the heparin is a unfractionated heparin or a fractioned heparin.

141. The method of claim 140, wherein the heparin is a fractioned heparin, and the fractioned heparin is a LMWH.

142. The method of claim 141, wherein the LMWH is selected from the group consisting of enoxaparin, dalteparin, reviparin, tinzaparin, nadroparin, certoparin, ardeparin, parnaparin, M118, M312, M405, M108, M115, and M411.

143. A method for in vivo delivery of a polysaccharide to a subject, comprising:
administering to a subject a therapeutically effective amount of a composition made by the method of claim 1 or 24, to thereby deliver the polysaccharide to the subject.

144. A method for oral delivery of heparin to a subject, comprising:
orally administering therapeutically effective amount of a heparin, to a subject, wherein the heparin has a net negative charge which is less than a reference net charge for the heparin,
to thereby delivery the heparin to the subject.

145. The method of claim 144, wherein the heparin is a LMWH.

146. The method of claim 145, wherein the reference net charge is the net charge of enoxaparin.

147. The method of claim 145, wherein the net charge of the LMWH is at least 30% less than the net charge of enoxaparin.

148. The method of claim 145, wherein the net charge of the LMWH is less than 18.

149. The method of claim 144, wherein the heparin further has a mass which less than a reference mass for the heparin.

150. The method of claim 149, wherein the heparin is a LMWH.

151. The method of claim 150, wherein the reference mass for the LMWH is selected from the group consisting of the mass of enoxaparin, the mass of nadroparin, the mass of dalteparin, the mass of reviparin, the mass of parnaparin, and the mass of tinzaparin.

152. The method of claim 151, wherein the mass of the LMWH is less than at least 30 Da than the mass of enoxaparin.

153. The method of claim 151, wherein the mass of the LMWH is less than at least 30 Da than the mass of nadroparin.

154. The method of claim 151, wherein the mass of the LMWH is less than at least 30 Da than the mass of dalteparin.

155. The method of claim 151, wherein the mass of the LMWH is less than at least 30 Da than the mass of reviparin.

156. The method of claim 151, wherein the mass of the LMWH is less than at least-30 Da than the mass of parnaparin.

157. The method of claim 151, wherein the mass of the LMWH is less than at least 30 Da than the mass of tinzaparin.

158. The method of claim 151, wherein the mass of the LMWH is about 4100 Da or less.

159. The method of claim 150, wherein the LMWH is derived from enoxaparin or unfractionated heparin and the enoxaparin or unfractionated heparin is neutralized and the mass reduced such that the LMWH has a charge to mass ratio of less than 19.32/4200.

160. The method of claim 150, wherein the LMWH is derived from nadroparin or unfractionated heparin and the nadroparin or unfractionated heparin is neutralized and the mass reduced such that the LMWH has a charge to mass ratio of less than 27.6/6000.

161. The method of claim 150, wherein the LMWH is derived from dalteparin or unfractionated heparin and the dalteparin or unfractionated heparin is neutralized and the mass reduced such that the LMWH has a charge to mass ratio of less than 23/5000.

162. The method of claim 150, wherein the LMWH is derived from reviparin or unfractionated heparin and the reviparin or unfractionated heparin is neutralized and the mass reduced such that the LMWH has a charge to mass ratio of less than 25.3/5500.

163. The method of claim 150, wherein the LMWH is derived from parnaparin or unfractionated heparin and the parnaparin or unfractionated heparin is neutralized and the mass reduced such that the LMWH has a charge to mass ratio of less than 30.4/6610.

164. The method of claim 150, wherein the LMWH is derived from tinzaparin or unfractionated heparin and the tinzaparin or unfractionated heparin is neutralized and the mass reduced such that the LMWH has a charge to mass ratio of less than 28.06/6100.

165. The method of claim 158, wherein the therapeutically effective amount of the LMWH is about 2 mg/kg or more.

166. The method of claim 158, wherein the LMWH has an absorption rate in the gastrointestinal tract of about 0.25 IU/ml or more over a period of about 1-5 hours.

167. The method of claim 158, wherein the at least 5% or more of the LMWH is delivered to the intestinal mucosa.

168. The method of claim 158, wherein the LMWH is delivered to the intestinal mucosa in an amount effective to produce a peak plasma concentration of the LWMH within 2 to 7 hours after delivery.

169. The method of claim 158, wherein 10% or more of the LMWH is detectable in the blood within about 2 to 7 hours after delivery.

170. The method of claim 158, wherein the bioavailability of the LMWH is at least about 10% or more.

171. A method for delivering M405 to a subject, comprising:
orally administering to a subject a composition comprising M405 in an effective amount to deliver a therapeutic dose of M405 to the subject, thereby delivering M405 to the subject.

172. A method for delivering M108 to a subject, comprising:
orally administering to a subject a composition comprising M108 in an effective amount to deliver a therapeutic dose of M108 to the subject, thereby delivering M108 to the subject.

173. A method for pulmonary delivery of heparin to a subject, comprising:
administering a therapeutically effective amount of a heparin to pulmonary tissue of a subject, wherein the heparin has a net negative charge which is less than a reference net charge for the heparin,
to thereby delivery the heparin to the subject.

174. The method of claim 173, wherein the heparin is a LMWH.

175. The method of claim 174, wherein the reference net charge is the net charge of enoxaparin.

176. The method of claim 175, wherein the net charge of the LMWH is at least 30% less than the net charge of enoxaparin.

177. The method of claim 176, wherein the net charge of the LMWH is less than 18.

178. The method of claim 173, wherein the heparin further has a mass which less than a reference mass for the heparin.

179. The method of claim 178, wherein the heparin is a LMWH.

180. The method of 179, wherein the reference mass for the LMWH is the mass of enoxaparin.

181. The method of claim 180, wherein the mass of the LMWH is less than at least 30 Da than the mass of enoxaparin.

182. The method of claim 179, wherein the reference mass is selected from the group consisting of the mass of nadroparin, the mass of dalteparin, the mass of reviparin, the mass of parnaparin, and the mass of tinzaparin.

183. The method of claim 182, wherein the reference mass is the mass of nadroparin and the mass of the LMWH is less than at least 30 Da than the mass of nadroparin.

184. The method of claim 182, wherein the reference mass is the mass of dalteparin and the mass of the LMWH is less than at least 30 Da than the mass of dalteparin.

185. The method of claim 182, wherein the reference mass is the mass of reviparin and the mass of the LMWH is less than at least 30 Da than the mass of reviparin.

186. The method of claim 182, wherein the reference mass is the mass of parnaparin and the mass of the LMWH is less than at least 30 Da than the mass of parnaparin.

187. The method of claim 182, wherein the reference mass is the mass of tinzaparin and the mass of the LMWH is less than at least 30 Da than the mass of tinzaparin.

188. The method of claim 180, wherein the mass of the LMWH is about 4100 Da or less.

189. The method of claim 180, wherein the LMWH is derived from enoxaparin or unfractionated heparin and the enoxaparin or unfractionated heparin is neutralized and the mass reduced such that the LMWH has a charge to mass ratio of less than 19.32/4200.

190. The method of claim 180, wherein the LMWH is derived from nadroparin or unfractionated heparin and the nadroparin or unfractionated heparin is neutralized and the mass reduced such that the LMWH has a charge to mass ratio of less than 27.6/6000.

191. The method of claim 180, wherein the LMWH is derived from dalteparin or unfractionated heparin and the dalteparin or unfractionated heparin is neutralized and the mass reduced such that the LMWH has a charge to mass ratio of less than 23/5000.

192. The method of claim 180, wherein the LMWH is derived from reviparin or unfractionated heparin and the reviparin or unfractionated heparin is neutralized and the mass reduced such that the LMWH has a charge to mass ratio of less than 25.3/5500.

193. The method of claim 180, wherein the LMWH is derived from parnaparin or unfractionated heparin and the parnaparin or unfractionated heparin is neutralized and the mass reduced such that the LMWH has a charge to mass ratio of less than 30.4/6610.

194. The method of claim 180, wherein the LMWH is derived from tinzaparin or unfractionated heparin and the tinzaparin or unfractionated heparin is neutralized and the mass reduced such that the LMWH has a charge to mass ratio of less than 28.06/6100.

195. The method of claim 174, wherein the LMWH has an absorption rate in the pulmonary tissue of about 0.25 IU/ml or more over a period of about 10 minutes to 5 hours.

196. The method of claim 174, wherein the at least 5% or more of the LMWH is delivered to the pulmonary tissue.

197. The method of claim 196, wherein at least 2% or more of the LMWH is delivered to the deep lung.

198. The method of claim 174, wherein the LMWH is delivered to the pulmonary tissue in an amount effective to produce a peak plasma concentration of the LWMH within 10 minutes to 3 hours after delivery.

199. The method of claim 174, wherein 2% or more of the LMWH is detectable in the blood within about 5 minutes to 5 hours after delivery.

200. The method of claim 174, wherein the therapeutically effective amount is at least 20 mg/puff.

201. The method of any of claims 138-200, wherein the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders; an allergy; a respiratory disorder, such as asthma, emphysema, adult respiratory distress syndrome (ARDS), cystic fibrosis, or lung reperfusion injury; a cancer or metastatic disorder; an angiogenic disorder, such as neovascular disorders of the eye, osteoporosis, psoriasis, arthritis, Alzheimer's, or is undergoing or having undergone surgical procedure, organ transplant, orthopedic surgery, hip replacement, knee replacement, treatment for a fracture, e.g., a hip fracture, percutaneous coronary intervention (PCI), stent placement, angioplasty, coronary artery bypass graft surgery (CABG).

202. A method for delivery of an HLGAG to the pulmonary system of a subject, comprising:
administering a therapeutically effective amount of an HLGAG to pulmonary tissue of a subject to provide a preselected effect in the subject wherein the dose of the HLGAG is at least 2 times greater than a subcutaneous or intravenous dose of the HLGAG which is effective to give the preselected therapeutic effect.

203. The method of claim 202, wherein the HLGAG is a synthetic HLGAG.

204. The method of claim 203, wherein the synthetic HLGAG is a synthetic pentasaccharide.

205. The method of claim 204, wherein the synthetic pentasaccharide is Arixtra.

206. The method of claim 202, wherein the preselected therapeutic effect is one or more of: anti-Xa activity and anti-IIa activity.

207. The method of claim 202, wherein the HLGAG is in a device which provides a therapeutically effective unit dose of the HLGAG.

208. The method of claim 207, wherein the HLGAG is a synthetic HLGAG and the therapeutically effective unit dose is about 0.06 mg/kg or more.

209. The method of claim 207, wherein the HLGAG is a synthetic HLGAG and the therapeutically effective unit dose is about 8 mg or more.

210. The method of claim 208 or 209, wherein the synthetic HLGAG is Arixtra or a derivative thereof.

211. The method of claim 208 or 209, wherein the synthetic HLGAG is one or more of the compounds provided in FIG. 9 and derivatives thereof.

212. The method of claim 207, wherein the HLGAG is a synthetic HLGAG and the therapeutically effective unit dose is amount effective to produce a peak plasma concentration of the HLGAG within about 5 minutes to about 5 hours after delivery.

213. The method of claim 212, wherein the synthetic HLGAG is Arixtra or a derivative thereof.

214. The methods of claim 202, wherein the HLGAG is in the form of a solid.

215. The method of claim 202, wherein the HLGAG has an absorption rate in the pulmonary tissue of at least about 0.25 IU/ml or more over a period of about 10 minutes to 5 hours.

216. The method of claim 215, wherein the HLGAG is a synthetic HLGAG.

217. The method of claim 216, wherein the synthetic HLGAG is Arixtra or derivatives thereof.

218. The method of claim 202, wherein at least 5% or more of the HLGAG is delivered to the upper and/or lower respiratory tract of the lung.

219. The method of claim 218, wherein the HLGAG is a synthetic HLGAG.

220. The method of claim 219, wherein the synthetic HLGAG is Arixtra or a derivative thereof.

221. The method of claim 202, wherein the HLGAG is in a composition and the composition further comprises a pharmaceutically acceptable carrier.

222. The method of claim 221, wherein the composition further comprises a delivery enhancer.

223. The method of claim 202, wherein the subject has or is at risk of a disorder selected from the group consisting of disease associated with coagulation, such as thrombosis, cardiovascular disease, vascular conditions or atrial fibrillation; migraine, atherosclerosis; an inflammatory disorder, such as autoimmune disease or atopic disorders;
an allergy; a respiratory disorder, such as asthma, emphysema, adult respiratory distress syndrome (ARDS), cystic fibrosis, or lung reperfusion injury; a cancer or metastatic disorder; an angiogenic disorder, a cardiovascular disorder, diabetes, obesity, osteoporosis, psoriasis, arthritis, Alzheimer's, a subject to undergo, undergoing or having undergone surgical procedure, organ transplant, orthopedic surgery, treatment for a fracture, e.g., a hip fracture, hip replacement, knee replacement, percutaneous coronary intervention (PCI), stent placement, angioplasty, and coronary artery bypass graft surgery (CABG).

224. A composition for pulmonary delivery, wherein the composition comprises a therapeutically effective amount of a synthetic HLGAG to provide a preselected effect, wherein the composition is in a device which delivers the HLGAG at a unit dose which is at least 2 times greater than the unit dose used for subcutaneous or intravenous delivery of the HLGAG to provide the preselected effect.

225. The composition of 224, wherein the preselected therapeutic effect is one or more of anti-Xa activity and anti-IIa activity.

226. The composition of claim 224, wherein the unit dose of the HLGAG to provide the preselected therapeutic effect is at least about 0.05 mg/kg.

227. The composition of claim 224, wherein unit dose of the HLGAG to provide the preselected therapeutic effect is at least about 16 mg.

228. The composition of claim 226 or 227, wherein the synthetic HLGAG is Arixtra or a derivative thereof.

229. The composition of claim 226 or 227, wherein the synthetic HLGAG is one or more of the synthetic HLGAGs of FIG. 9 and derivatives thereof.

230. The composition of claim 228, wherein the preselected therapeutic effect is one or more of anti-Xa activity and anti-IIa activity.

231. The composition of claim 229, wherein the preselected therapeutic effect is one or more of anti-Xa activity and anti-IIa activity.

232. The composition of claim 224, further comprising a surfactant.

233. A pressurized container or dispenser comprising the composition of claim 224, 230 or 231.

234. The pressurized container or dispenser of claim 233, wherein the pressurized container or dispenser comprises one or more of a suitable propellant and a nebulizer.
